(19) United States
(12) Patent Application Publication
(10) Pub. No.: US 2024/0279713 A1
(43) Pub. Date: Aug. 22, 2024

(54) DEGENERATE OLIGONUCLEOTIDES AND THEIR USES

(71) Applicant: SIGMA-ALDRICH CO. LLC, St. Louis, MO (US)

(72) Inventors: Brian Ward, St. Louis, MO (US); Kenneth Heuermann, Kirkwood, MO (US)

(21) Appl. No.: 18/470,565

(22) Filed: Sep. 20, 2023

Related U.S. Application Data
(63) Continuation of application No. 17/354,443, filed on Jun. 22, 2021, now abandoned.
Application No. 17/354,443 is a continuation of application No. 16/834,141, filed on Mar. 30, 2020, now abandoned.
Application No. 16/834,141 is a continuation of application No. 16/276,530, filed on Feb. 14, 2019, now abandoned.
Application No. 16/276,530 is a continuation of application No. 14/483,875, filed on Sep. 11, 2014, now abandoned.
Application No. 14/483,875 is a division of application No. 11/872,272, filed on Oct. 15, 2007, now abandoned.

(51) Int. Cl. C12Q 1/6806; C12N 15/10; C12Q 1/6853; C12Q 1/6876

(52) U.S. Cl. CPC C12Q 1/6806; C12N 15/1093; C12Q 1/6853; C12Q 1/6876; C12Q 2525/179

(57) ABSTRACT

The present invention provides a plurality of oligonucleotides comprising a semi-random sequence, wherein the semi-random sequence comprises degenerate nucleotides that are substantially non-complementary. Also provided are methods for using the plurality of oligonucleotides to amplify a population of target nucleic acids.

### CROSS-REFERENCE TO RELATED APPLICATIONS

This application is a continuation of U.S. patent application Ser. No. 17/354,443, filed Jun. 22, 2021, which is a continuation of U.S. patent application Ser. No. 16/834, 141, filed Mar. 30, 2020, which is a continuation of U.S. patent application Ser. No. 16/276,530, filed Feb. 14, 2019, which is a continuation of U.S. patent application Ser. No. 14/483,875, filed Sep. 11, 2014, which is a divisional of U.S. patent application Ser. No. 11/872,272, filed Oct. 15, 2007, each of which is incorporated by reference herein in their entirety.

### SEQUENCE LISTING

The present application contains a Sequence Listing which has been filed electronically in XML format and is hereby incorporated by reference in its entirety. Said XML copy, created on April 8, 2024, is named SIG103USCNT4_SL_ST26.xml and is 58,367 bytes in size.

### FIELD OF THE INVENTION

The present invention relates to a plurality of oligonucleotides comprising a semi-random sequence. In particular, the semi-random sequence comprises degenerate nucleotides that are substantially non-complementary. Furthermore, the degenerate oligonucleotides may be used to amplify a population of target nucleic acids.

### BACKGROUND OF THE INVENTION

In many fields of research and diagnostics, the types of analyses that can be performed are limited by the quantity of available nucleic acids. Because of this, a variety of techniques have been developed to amplify small quantities of nucleic acids. Among these are whole genome amplification (WGA) and whole transcriptome amplification (WTA) procedures, which are non-specific amplification techniques designed to provide an unbiased representation of the entire starting genome or transcriptome.

Many of these amplification techniques utilize degenerate oligonucleotide primers in which each oligonucleotide comprises a random sequence (i.e., each nucleotide may be any nucleotide) or a non-complementary variable sequence (i.e., each nucleotide may be either of two non-complementary nucleotides). Whereas random primer complementarity results in excessive primer-dimer formation, amplification utilizing non-complementary variable primers, having reduced sequence complexity, is characterized by incomplete coverage of the starting population of nucleic acids.

Thus, there is a need for oligonucleotide primers that are substantially non-complementary while still having a high degree of sequence diversity. Such primers would be able to hybridize to a maximal number of sequences throughout the target nucleic acid, while the tendency to self-hybridize or cross-hybridize with other primers would be minimized. Such primers would be extremely useful in WGA or WTA techniques.

### SUMMARY OF THE INVENTION

One aspect of the present invention provides a method for amplifying a population of target nucleic acids. The method comprises contacting the population of target nucleic acids with a plurality of oligonucleotide primers to form a plurality of nucleic acid-primer duplexes. Each of the oligonucleotide primers comprises the formula NₘXₚZ_{q}, wherein N, X, and Z are degenerate nucleotides, as defined above, and m, p, and q are integers. In particular, m either is 0 or is from 2 to 20, and p and q are from 0 to 20, provided, however, that no two integers are 0, and further provided that oligonucleotides comprising N, which have at least two N residues, have at least one X or Z residue separating the two N residues. The method further comprises replicating the plurality of nucleic acid-primer duplexes to create a library of replicated strands. Furthermore, the amount of replicated strands in the library exceeds the amount of starting target nucleic acids, which indicates amplification of the population of target nucleic acids.

Other aspects and features of the invention are described in more detail herein.

### DESCRIPTION OF THE FIGURES

FIG. 1 illustrates real-time quantitative PCR of amplified cDNA and unamplified cDNA. The deltaC(t) values for each primer set are plotted for unamplified cDNA (light gray bars), D-amplified cDNA (dark gray bars), and K-amplified cDNA (white bars).

FIG. 2A illustrates a microarray analysis of amplified cDNA and unamplified cDNA. Log base 2 ratios of D-amplified cDNA targets are plotted against the log base 2 ratio for unamplified cDNA targets.

FIG. 2B illustrates a microarray analysis of K-amplified cDNA and unamplified DNA. Log base 2 ratios of K-amplified cDNA targets are plotted against the log base 2 ratio for unamplified cDNA targets.

FIG. 3 presents agarose gel images of WTA products amplified from NaOH-degraded RNA with preferred interrupted N library synthesis primers or control primers (1K9 and 1D9). The molecular size standards (in bp) that were loaded on each gel are presented on left, and the times (in minutes) of RNA exposure to NaOH are presented on the right.

FIG. 4 presents agarose gel images of WTA products amplified with preferred interrupted N library synthesis primers or control primers (1K9 and 1D9). Library synthesis was performed in the presence (+) or absence (−) of RNA, and with either MMLV reverse transcriptase (M) or MMLV reverse transcriptase and Klenow exo-minus DNA polymerase (MK). Library amplification was catalyzed by either JUMPSTART™ Taq DNA polymerase (JST) or KLENTAQ™ DNA polymerase (KT). The molecular size standards (in bp) that were loaded on each gel are presented on left, and the different reaction conditions are indicated on the right.

FIG. 5 presents agarose gel images of WTA products amplified with the five most preferred interrupted N library synthesis primers, various combinations of the preferred primers, or control primers. Library synthesis was performed with various concentrations of each primer or primer set. The primer concentrations (10, 2, 0.4, or 0.08 μM, from left to right) are diagrammed by triangles at the top of the images. The primer(s) within a given set are listed to the right of the images.

### DETAILED DESCRIPTION OF THE INVENTION

It has been discovered that oligonucleotides comprising a mixture of 4-fold degenerate nucleotides, 3-fold degenerate nucleotides, and/or 2-fold degenerate nucleotides have reduced intramolecular and/or intermolecular interactions, while retaining adequate sequence diversity for the representative amplification of a target nucleic acid. These oligonucleotides comprising semi-random regions are able to hybridize to many sequences throughout the target nucleic acid and provide many priming sites for replication and amplification of the target nucleic acid. At the same time, however, these oligonucleotides generally neither self-hybridize to form primer secondary structures nor cross-hybridize to form primer-dimer pairs.

### (I) Plurality of Oligonucleotides

One aspect of the present invention encompasses a plurality of oligonucleotides comprising a semi-random sequence. The semi-random sequence of the oligonucleotides comprises nucleotides that are substantially non-complementary, thereby reducing intramolecular and intermolecular interactions for the plurality of oligonucleotides. The semi-random sequence of the oligonucleotides, however, still provides substantial sequence diversity to permit hybridization to a maximal number of sequences contained within a target population of nucleic acids. The oligonucleotides of the invention may further comprise a non-random sequence.

### (a) Semi-random Sequence

The semi-random sequence of the plurality of oligonucleotides comprises degenerate nucleotides (see Table A). A degenerate nucleotide may have 2-fold degeneracy (i.e., it may be one of two nucleotides), 3-fold degeneracy (i.e., it may one of three nucleotides), or 4-fold degeneracy (i.e., it may be one of four nucleotides). Because the oligonucleotides of the invention are degenerate, they are mixtures of similar, but not identical, oligonucleotides. The total degeneracy of a oligonucleotide may be calculated as follows:

$$Degeneracy = {2}^{a} \times {3}^{b} \times {4}^{c}$$

wherein “a” is the total number 2-fold degenerate nucleotides (previously defined as Z, above), “b” is the total number of 3-fold degenerate nucleotides (previously defined as X, above), and “c” is the total number of 4-fold nucleotides (previously defined as N, above).

Degenerate nucleotides may be complementary, non-complementary, or partially non-complementary (see Table A). Complementarity between nucleotides refers to the ability to form a Watson-Crick base pair through specific hydrogen bonds (e.g., A and T base pair via two hydrogen bonds; and C and G are base pair via three hydrogen bonds).

TABLE A
Degenerate Nucleotides.

| Symbol | Origin of Symbol | Meaning* | Complementarity |
|---|---|---|---|
| K | keto | G or T/U | Non-complementary |
| M | amino | A or C | Non-complementary |
| R | purine | A or G | Non-complementary |
| Y | pyrimidine | C or T/U | Non-complementary |
| S | strong interactions | C or G | Complementary |
| W | weak interactions | A or T/U | Complementary |
| B | not A | C or G or T/U | Partially non- |
| | | | complementary |
| D | not C | A or G or T/U | Partially non- |
| | | | complementary |
| H | not G | A or C or T/U | Partially non- |
| | | | complementary |
| V | not T/U | A or C or G | Partially non- |
| | | | complementary |
| N | any | A or C or G or T/U | Complementary |

*A = adenosine, C = cytidine, G = guanosine, T = thymidine, U = uridine

The term “oligonucleotide,” as used herein, refers to a molecule comprising two or more nucleotides. The nucleotides may be deoxyribonucleotides or ribonucleotides. The oligonucleotides may comprise the standard four nucleotides (i.e., A, C, G, and T/U), as well as nucleotide analogs. A nucleotide analog refers to a nucleotide having a modified purine or pyrimidine base and/or a modified ribose moiety. A nucleotide analog may be a naturally occurring nucleotide (e.g., inosine) or a non-naturally occurring nucleotide. Non-limiting examples of modifications on the sugar or base moieties of a nucleotide include the addition (or removal) of acetyl groups, amino groups, carboxyl groups, carboxymethyl groups, hydroxyl groups, methyl groups, phosphoryl groups, and thiol groups, as well as the substitution of the carbon and nitrogen atoms of the bases with other atoms (e.g., 7-deaza purines). Nucleotide analogs also include dideoxy nucleotides, 2′-O-methyl nucleotides, locked nucleic acids (LNA), peptide nucleic acids (PNA), and morpholinos. The backbone of the oligonucleotides may comprise phosphodiester linkages, as well as phosphothioate, phosphoramidite, or phosphorodiamidate linkages.

The plurality of oligonucleotides of the invention comprise the formula NₘXₚZ_{q}, wherein:
N is a 4-fold degenerate nucleotide selected from the group consisting of adenosine (A), cytidine (C), guanosine (G), and thymidine/uridine (T/U);
X is a 3-fold degenerate nucleotide selected from the group consisting of B, D, H, and V, wherein B is selected from the group consisting of C, G, and T/U; D is selected from the group consisting of A, G, and T/U; H is selected from the group consisting of A, C, and T/U; and V is selected from the group consisting of A, C, and G;
Z is a 2-fold degenerate nucleotide selected from the group consisting of K, M, R, and Y, wherein K is selected from the group consisting of G and T/U; M is selected from the group consisting of A and C; R is selected from the group consisting of A and G; and Y is selected from the group consisting of C and T/U; and
m, p, and q are integers, m either is 0 or is from 2 to 20, p and q are from 0 to 20; provided, however, that either no two integers are 0 or both m and q are 0, and further provided that oligonucleotides comprising N, which have at least two N residues, have at least one X or Z residue separating the two N residues.

The plurality of oligonucleotides comprise complementary 4-fold degenerate nucleotides and/or partially non-complementary 3-fold degenerate nucleotides and/or non-complementary 2-fold degenerate nucleotides. Furthermore, in oligonucleotides containing N residues, the at least two N residues are separated by at least one X or Z residue. Thus, partially non-complementary 3-fold degenerate nucleotides and/or non-complementary 2-fold degenerate nucleotides interrupt the complementary N residues. The oligonucleotides of the invention, therefore, are substantially non-complementary.

In some embodiments, in which no two integers of the formula NₘXₚZ_{q} are zero, the plurality of oligonucleotides may, therefore, comprise either formula N₂₋₂₀X₁₋₂₀Z₁₋₂₀ (or NXZ), formula N₀X₁₋₂₀Z₁₋₂₀ (or XZ), formula N₂₋₂₀X₀Z₁₋₂₀ (or NZ), or formula N₂₋₂₀X₁₋₂₀Z₀ (or NX) (see Table B for specific formulas). Accordingly, oligonucleotides comprising formula NXZ, may range from about 4 nucleotides to about 60 nucleotides in length. More specifically, oligonucleotides comprising formula NXZ may range from about 48 nucleotides to about 60 nucleotides in length, from about 36 nucleotides to about 48 nucleotides in length, from about 24 nucleotides to about 36 nucleotides in length, from about 14 nucleotides to about 24 nucleotides in length, or from about 4 nucleotides to about 14 nucleotides in length. Oligonucleotides comprising formula XZ may range from about 2 nucleotides to about 40 nucleotides in length. More specifically, oligonucleotides comprising this formula may range from about 24 nucleotides to about 40 nucleotides in length, from about 14 nucleotides to about 24 nucleotides in length, or from about 2 nucleotides to about 14 nucleotides in length. Lastly, oligonucleotides comprising formula NZ or formula NX may range from about 3 nucleotides to about 40 nucleotides in length. More specifically, oligonucleotides comprising these formulas may range from about 24 nucleotides to about 40 nucleotides in length, from about 14 nucleotides to about 24 nucleotides in length, or from about 3 nucleotides to about 14 nucleotides in length.

TABLE B
Exemplary oligonucleotide formulas.

| NXZ | XZ | NZ | NX |
|---|---|---|---|
| NBK | BK | NK | NB |
| NBM | BM | NM | ND |
| NBR | BR | NR | NH |
| NBY | BY | NY | NV |
| NDK | DK | | |
| NDM | DM | | |
| NDR | DR | | |
| NDY | DY | | |
| NHK | HK | | |
| NHM | HM | | |
| NHR | HR | | |
| NHY | HY | | |
| NVK | VK | | |
| NVM | VM | | |
| NVR | VR | | |
| NVY | VY | | |

In an alternate embodiment, the plurality of oligonucleotides may comprise the formula NₘXₚ, wherein N and X are nucleotides as defined above, m ranges from 2 to 13, p ranges from 1 to 12, the sum total of m and p is 14, and the at least two N residues are separated by at least one X residue. In another embodiment, the plurality of oligonucleotides may comprise the formula NₘXₚ, wherein N and X are nucleotides as defined above, m ranges from 2 to 12, p ranges from 1 to 11, the sum total of m and p is 13, and the at least two N residues are separated by at least one X residue. In still another embodiment, the plurality of oligonucleotides may comprise the formula NₘXₚ, wherein N and X are nucleotides as defined above, m ranges from 2 to 11, p ranges from 1 to 10, the sum total of m and p is 12, and the at least two N residues are separated by at least one X residue. In another embodiment, the plurality of oligonucleotides may comprise the formula NₘXₚ, wherein N and X are nucleotides as defined above, m ranges from 2 to 10, p ranges from 1 to 9, the sum total of m and p is 11, and the at least two N residues are separated by at least one X residue. In yet another embodiment, the plurality of oligonucleotides may comprise the formula NₘXₚ, wherein N and X are nucleotides as defined above, m ranges from 2 to 9, p ranges from 1 to 8, the sum total of m and p is 10, and the at least two N residues are separated by at least one X residue. In still another embodiment, the plurality of oligonucleotides may comprise the formula NₘXₚ, wherein N and X are nucleotides as defined above, m ranges from 2 to 7, p ranges from 1 to 6, the sum total of m and p is 8, and the at least two N residues are separated by at least one X residue. In another embodiment, the plurality of oligonucleotides may comprise the formula NₘXₚ, wherein N and X are nucleotides as defined above, m ranges from 2 to 6, p ranges from about 1 to 5, the sum total of m and p is 7, and the at least two N residues are separated by at least one X residue. In yet another embodiment, the plurality of oligonucleotides may comprise the formula NₘXₚ, wherein N and X are nucleotides as defined above, m ranges from 2 to 5, p ranges from 1 to 4, the sum total of m and p is 6, and the at least two N residues are separated by at least one X residue. In a preferred embodiment, the plurality of oligonucleotides may comprise the formula NₘXₚ, wherein N and X are nucleotides as defined above, m ranges from 2 to 8, p ranges from 1 to 7, the sum total of m and p is 9, and the at least two N residues are separated by at least one X residue. Table C presents (5′ to 3′) sequences of this preferred embodiment, i.e., a 9-nucleotide long semi-random region.

TABLE C
Nucleotide sequences (5′ to 3′) of an exemplary semi-random region.

| | | | | | |
|---|---|---|---|---|---|
| XXXXXXNXN | XXNNXXNNX | XNXNNNXNN | NXXXNXXXN | NXNXNNNNN | NNXNXNNNX |
| XXXXXNXXN | XXNNXXNNN | XNXNNNNXX | NXXXNXXNX | NXNNXXXXX | NNXNXNNNN |
| XXXXXNXNX | XXNNXNXXX | XNXNNNNXN | NXXXNXXNN | NXNNXXXXN | NNXNNXXXX |
| XXXXXNXNN | XXNNXNXXN | XNXNNNNNX | NXXXNXNXX | NXNNXXXNX | NNXNNXXXN |
| XXXXXNNXN | XXNNXNXNX | XNXNNNNNN | NXXXNXNXN | NXNNXXXNN | NNXNNXXNX |
| XXXXNXXXN | XXNNXNXNN | XNNXXXXXN | NXXXNXNNX | NXNNXXNXX | NNXNNXXNN |
| XXXXNXXNX | XXNNXNNXX | XNNXXXXNX | NXXXNXNNN | NXNNXXNXN | NNXNNXNXX |
| XXXXNXXNN | XXNNXNNXN | XNNXXXXNN | NXXXNNXXX | NXNNXXNNX | NNXNNXNXN |
| XXXXNXNXX | XXNNXNNNX | XNNXXXNXX | NXXXNNXXN | NXNNXXNNN | NNXNNXNNX |
| XXXXNXNXN | XXNNXNNNN | XNNXXXNXN | NXXXNNXNX | NXNNXNXXX | NNXNNXNNN |
| XXXXNXNNX | XXNNNXXXN | XNNXXXNNX | NXXXNNXNN | NXNNXNXXN | NNXNNNXXX |
| XXXXNXNNN | XXNNNXXNX | XNNXXXNNN | NXXXNNNXX | NXNNXNXNX | NNXNNNXXN |
| XXXXNNXXN | XXNNNXXNN | XNNXXNXXX | NXXXNNNXN | NXNNXNXNN | NNXNNNXNX |
| XXXXNNXNX | XXNNNXNXX | XNNXXNXXN | NXXXNNNNX | NXNNXNNXX | NNXNNNXNN |
| XXXXNNXNN | XXNNNXNXN | XNNXXNXNX | NXXXNNNNN | NXNNXNNXN | NNXNNNNXX |
| XXXXNNNXN | XXNNNXNNX | XNNXXNXNN | NXXNXXXXX | NXNNXNNNX | NNXNNNNXN |
| XXXNXXXXX | XXNNNXNNN | XNNXXNNXX | NXXNXXXXN | NXNNXNNNN | NNXNNNNNX |
| XXXNXXXXN | XXNNNNXXN | XNNXXNNXN | NXXNXXXNX | NXNNNXXXX | NNXNNNNNN |
| XXXNXXXNX | XXNNNNXNX | XNNXXNNNX | NXXNXXXNN | NXNNNXXXN | NNNXXXXXN |
| XXXNXXXNN | XXNNNNXNN | XNNXXNNNN | NXXNXXNXX | NXNNNXXNX | NNNXXXXNX |
| XXXNXXNXX | XXNNNNNXN | XNNXNXXXX | NXXNXXNXN | NXNNNXXNN | NNNXXXXNN |
| XXXNXXNXN | XNXXXXXXN | XNNXNXXXN | NXXNXXNNX | NXNNNXNXX | NNNXXXNXX |
| XXNXXXNNX | XNXXXXXNX | XNNXNXXNX | NXXNXXNNN | NXNNNXNXN | NNNXXXNXN |
| XXXNXXNNN | XNXXXXXNN | XNNXNXXNN | NXXNXNXXX | NXNNNXNNX | NNNXXXNNX |
| XXXNXNXXX | XNXXXXNXX | XNNXNXNXX | NXXNXNXXN | NXNNNXNNN | NNNXXXNNN |
| XXXNXNXXN | XNXXXXNXN | XNNXNXNXN | NXXNXNXNX | NXNNNNXXX | NNNXXNXXX |
| XXXNXNXNX | XNXXXXNNX | XNNXNXNNX | NXXNXNXNN | NXNNNNXXN | NNNXXNXXN |
| XXXNXNXNN | XNXXXXNNN | XNNXNXNNN | NXXNXNNXX | NXNNNNXNX | NNNXXNXNX |
| XXXNXNNXX | XNXXXNXXX | XNNXNNXXX | NXXNXNNXN | NXNNNNXNN | NNNXXNXNN |
| XXXNXNNXN | XNXXXNXXN | XNNXNNXXN | NXXNXNNNX | NXNNNNNXX | NNNXXNNXX |
| XXXNXNNNX | XNXXXNXNX | XNNXNNXNX | NXXNXNNNN | NXNNNNNXN | NNNXXNNXN |
| XXXNXNNNN | XNXXXNXNN | XNNXNNXNN | NXXNNXXXX | NXNNNNNNX | NNNXXNNNX |
| XXXNNXXXN | XNXXXNNXX | XNNXNNNXX | NXXNNXXXN | NXNNNNNNN | NNNXXNNNN |
| XXXNNXXNX | XNXXXNNXN | XNNXNNNXN | NXXNNXXNX | NNXXXXXXN | NNNXNXXXX |
| XXXNNXXNN | XNXXXNNNX | XNNXNNNNX | NXXNNXXNN | NNXXXXXNX | NNNXNXXXN |
| XXXNNXNXX | XNXXXNNNN | XNNXNNNNN | NXXNNXNXX | NNXXXXXNN | NNNXNXXNX |
| XXXNNXNXN | XNXXNXXXX | XNNNXXXXN | NXXNNXNXN | NNXXXXNXX | NNNXNXXNN |
| XXXNNXNNX | XNXXNXXXN | XNNNXXNXX | NXXNNXNNX | NNXXXXNXN | NNNXNXNXX |
| XXNNNXNNN | XNXXNXXNX | XNNNXXXNN | NXXNNXNNN | NNXXXXNNX | NNNXNXNXN |
| XXXNNNXXN | XNXXNXXNN | XNNNXXNXX | NXXNNNXXX | NNXXXXNNN | NNNXNXNNX |
| XXXNNNXNX | XNXXNXNXX | XNNNXXNXN | NXXNNNXXN | NNXXXNXXX | NNNXNXNNN |
| XXXNNNXNN | XNXXNXNXN | XNNNXXNNX | NXXNNNXNX | NNXXXNXXN | NNNXNNXXX |
| XXXNNNNXN | XNXXNXNNX | XNNNXXNNN | NXXNNNXNN | NNXXXNXNX | NNNXNNXXN |
| XXNXXXXXN | XNXXNXNNN | XNNNXNXXX | NXXNNNNXX | NNXXXNXNN | NNNXNNXNX |
| XXNXXXXNX | XNXXNNXXX | XNNNXNXXN | NXXNNNNXN | NNXXXNNXX | NNNXNNXNN |
| XXNXXXXNN | XNXXNNXXN | XNNNXNXNX | NXXNNNNNX | NNXXXNNXN | NNNXNNNXX |
| XXNXXXNXX | XNXXNNXNX | XNNNXNXNN | NXXNNNNNN | NNXXXNNNX | NNNXNNNXN |
| XXNXXXNXN | XNXNNXNNN | XNNNXNNXX | NXNXXXXXX | NNXXXNNNN | NNNXNNNNX |
| XXNXXXNNX | XNXXNNNXX | XNNNXNNXN | NXNXXXXXN | NNXXNXXXX | NNNXNNNNN |
| XXNXXXNNN | XNXXNNNXN | XNNNXNNNX | NXNXXXXNX | NNXXNXXXN | NNNNXXXXX |
| XXNXXNXXX | XNXXNNNNX | XNNNXNNNN | NXNXXXXNN | NNXXNXXNX | NNNNXXXXN |
| XXNXXNXXN | XNXXNNNNN | XNNNNXXXN | NXNXXXNXX | NNXXNXXNN | NNNNXXXNX |
| XXNXXNXNX | XNXNXXXXX | XNNNNXXNX | NXNXXXNXN | NNXXNXNXX | NNNNXXXNN |
| XXNXXNXNN | XNXNXXXXN | XNNNNXXNN | NXNXXXNNX | NNXXNXNXN | NNNNXXNXX |
| XXNXXNNXX | XNXNXXXNX | XNNNNXNXX | NXNXXXNNN | NNXXNXNNX | NNNNXXNXN |
| XXNXXNNXN | XNXNXXXNN | XNNNNXNXN | NXNXXNXXX | NNXXNXNNN | NNNNXXNNX |
| XXNXXNNNX | XNXNXXNXX | XNNNNXNNX | NXNXXNXXN | NNXXNNXXX | NNNNXXNNN |
| XXNXXNNNN | XNXNXXNXN | XNNNNXNNN | NXNXXNXNX | NNXXNNXXN | NNNNXNXXX |
| XXNXNXXXX | XNXNXXNNX | XNNNNNXXN | NXNXXNXNN | NNXXNNXNX | NNNNXNXXN |
| XXNXNXXXN | XNXNXXNNN | XNNNNNXNX | NXNXXNNXX | NNXXNNXNN | NNNNXNXNX |
| XXNXNXXNX | XNXNXNXXX | XNNNNNXNN | NXNXXNNXN | NNXXNNNXX | NNNNXNXNN |
| XXNXNXXNN | XNXNXNXXN | XNNNNNNXN | NXNXXNNNX | NNXXNNNXN | NNNNXNNXX |
| XXNXNXNXX | XNXNXNXNX | NXXXXXXXN | NXNXXNNNN | NNXXNNNNX | NNNNXNNXN |
| XXNXNXNXN | XNXNXNXNN | NXXXXXXNX | NXNXNXXXX | NNXXNNNNN | NNNNXNNNX |
| XXNXNXNNX | XNXNXNNXX | NXXXXXXNN | NXNXNXXXN | NNXNXXXXX | NNNNXNNNN |
| XXNXNXNNN | XNXNXNNXN | NXXXXXNXX | NXNXNXXNX | NNXNXXXXN | NNNNNXXXX |
| XXNXNNXXX | XNXNXNNNX | NXXXXXNXN | NXNXNXXNN | NNXNXXXNX | NNNNNXXXN |
| XXNXNNXXN | XNXNXNNNN | NXXXXXNNX | NXNXNXNXX | NNXNXXXNN | NNNNNXXNX |
| XXNXNNXNX | XNXNNXXXX | NXXXXXNNN | NXNXNXNXN | NNXNXXNXX | NNNNNXXNN |
| XXNXNNXNN | XNXNNXXXN | NXXXXNXXX | NXNXNXNNX | NNXNXXNXN | NNNNNXNXX |
| XXNXNNNXX | XNXNNXXNX | NXXXXNXXN | NXNXNXNNN | NNXNXXNNX | NNNNNXNXN |
| XXNXNNNXN | XNXNNXXNN | NXXXXNXNX | NXNXNNXXX | NNXNXXNNN | NNNNNXNNX |
| XXNXNNNNX | XNXNNXNXX | NXXXXNXNN | NXNXNNXXN | NNXNXNXXX | NNNNNXNNN |
| XXNXNNNNN | XNXNNXNXN | NXXXXNNXX | NXNXNNXNX | NNXNXNXXN | NNNNNNXXX |
| XXNNXXXXN | XNXNNXNNX | NXXXXNNXN | NXNXNNXNN | NNXNXNXNX | NNNNNNXXN |
| XXNNXXXNX | XNXNNXNNN | NXXXXNNNX | NXNXNNNXX | NNXNXNXNN | NNNNNNXNX |
| XXNNXXXNN | XNXNNNXXX | NXXXXNNNN | NXNXNNNXN | NNXNXNNXX | NNNNNNXNN |
| XXNNXXNXX | XNXNNNXXN | NXXXNXXXX | NXNXNNNNX | NNXNXNNXN | NNNNNNNXN |
| XXNNXXNXN | XNXNNNXNX | | | | |

In still another alternate embodiment, the plurality of oligonucleotides may comprise formula NₘXₚ, wherein N and X are nucleotides as defined above, m ranges from 2 to 13, p ranges from 1 to 12, and the sum total of m and p ranges from 6 to 14, the at least two N residues are separated by at least one X residue, and there are no more than three consecutive N residues. In this embodiment, therefore, partially non-complementary 3-fold degenerate nucleotides are interspersed throughout the sequence such that there are no long runs (≥4) of the complementary 4-fold degenerate nucleotide (N). In general, such a design may reduce self-hybridization and/or cross-hybridization within the plurality of oligonucleotides. In an exemplary embodiment, the plurality of oligonucleotides may comprise formula NₘXₚ, wherein N and X are nucleotides as defined above, m ranges from 2 to 8, p ranges from 1 to 7, and the sum total of m and p is 9, the at least two N residues are separated by at least one X residue, and there are no more than three consecutive N residues. Table D lists the (5′ to 3′) sequences of this preferred embodiment, i.e., a 9-nucleotide long semi-random region containing no more that three consecutive N residues.

TABLE D
Nucleotide sequences (5′ to 3′) of an exemplary semi-random region having
no more than 3 consecutive N residues.

| | | | | | |
|---|---|---|---|---|---|
| XXXXXXNXN | XXNXNNXXX | XNXNXNXXX | NXXXXXXNX | NXNXXNXXN | NNXXNXNNX |
| XXXXXNXXN | XXNXNNXXN | XNXNXNXXN | NXXXXXXNN | NXNXXNXNX | NNXXNXNNN |
| XXXXXNXNX | XXNXNNXNX | XNXNXNXNX | NXXXXXNXX | NXNXXNXNN | NNXXNNXXX |
| XXXXXNXNN | XXNXNNXNN | XNXNXNXNN | NXXXXXNXN | NXNXXNNXX | NNXXNNXXN |
| XXXXXNNXN | XXNXNNNXX | XNXNXNNXX | NXXXXXNNX | NXNXXNNXN | NNXXNNXNX |
| XXXXNXXXN | XXNXNNNXN | XNXNXNNXN | NXXXXXNNN | NXNXXNNNX | NNXXNNXNN |
| XXXXNXXNX | XXNNXXXXN | XNXNXNNNX | NXXXXNXXX | NXNXNXXXX | NNXXNNNXX |
| XXXXNXXNN | XXNNXXXNX | XNXNNXXXX | NXXXXNXXN | NXNXNXXXN | NNXXNNNXN |
| XXXXNXNXX | XXNNXXXNN | XNXNNXXXN | NXXXXNXNX | NXNXNXXNX | NNXNXXXXX |
| XXXXNXNXN | XXNNXXNXX | XNXNNXXNX | NXXXXNXNN | NXNXNXXNN | NNXNXXXXN |
| XXXXNXNNX | XXNNXXNXN | XNXNNXXNN | NXXXXNNXX | NXNXNXNXX | NNXNXXXNX |
| XXXXNXNNN | XXNNXXNNX | XNXNNXNXX | NXXXXNNXN | NXNXNXNXN | NNXNXXXNN |
| XXXXNNXXN | XXNNXXNNN | XNXNNXNXN | NXXXXNNNX | NXNXNXNNX | NNXNXXNXX |
| XXXXNNXNX | XXNNXNXXX | XNXNNXNNX | NXXXNXXXX | NXNXNXNNN | NNXNXXNXN |
| XXXXNNXNN | XXNNXNXXN | XNXNNXNNN | NXXXNXXXN | NXNXNNXXX | NNXNXXNNX |
| XXXXNNNXN | XXNNXNXNX | XNXNNNXXX | NXXXNXXNX | NXNXNNXXN | NNXNXXNNN |
| XXXNXXXXX | XXNNXNXNN | XNXNNNXXN | NXXXXNXNN | NXNXNNXNX | NNXNXNXXX |
| XXXNXXXXN | XXNNXNNXX | XNXNNNXNX | NXXXNXNXX | NXNXNNXNN | NNXNXNXXN |
| XXXNXXXNX | XXNNXNNXN | XNXNNNXNN | NXXXNXNXN | NXNXNNNXX | NNXNXNXNX |
| XXXNXXXNN | XXNNXNNNX | XNNXXXXXN | NXXXNXNNX | NXNXNNNXN | NNXNXNXNN |
| XXXNXXXNX | XXNNNXXXN | XNNXXXXNX | NXXXNXNNN | NXNNXXXXX | NNXNXNNXX |
| XXXNXXNXN | XXNNNXXNX | XNNXXXXNN | NXXXNNXXX | NXNNXXXXN | NNXNXNNXN |
| XXXNXXNNX | XXNNNXXNN | XNNXXXNXX | NXXXNNXXN | NXNNXXXNX | NNXNXNNNX |
| XXXNXXNNN | XXNNNXNXX | XNNXXXNXN | NXXXNNXNX | NXNNXXXNN | NNXNNXXXX |
| XXXNXNXXX | XXNNNXNXN | XNNXXXNNX | NXXXNNXNN | NXNNXXNXX | NNXNNXXXN |
| XXXNXNXXN | XXNNNXNNX | XNNXXXNNN | NXXNNNXXX | NXNNXXNXN | NNXNNXXNX |
| XXXNXNXNX | XXNNNXNNN | XNNXXXNXX | NXXNNNXXN | NXNNXXNNX | NNXNNXXNN |
| XXXNXNXNN | XNXXXXXXN | XNNXXXNXN | NXXNXXXXX | NXNNXXNNN | NNXNNXNXX |
| XXXNXNNXX | XNXXXXXNX | XNNXXNXNX | NXXNXXXXN | NXNNXNXXX | NNXNNXNXN |
| XXXNXNNXN | XNXXXXXNN | XNNXXNXNN | NXXNXXXNX | NXNNXNXXN | NNXNNXNNX |
| XXXNXNNNX | XNXXXXNXX | XNNXXNNXX | NXXNXXXNN | NXNNXNXNX | NNXNNXNNN |
| XXXNNXXXN | XNXXXXNXN | XNNXXNNXN | NXXNXXNXX | NXNNXNXNN | NNXNNNXXX |
| XXXNNXXNX | XNXXXXNNX | XNNXXNNNX | NXXNXXNXN | NXNNXNNXX | NNXNNNXXN |
| XXXNNXXNN | XNXXXXNNN | XNNXNXXXX | NXXNXXNNX | NXNNXNNXN | NNXNNNXNX |
| XXXNNXNXX | XNXXXNXXX | XNNXNXXXN | NXXNXXNNN | NXNNXNNNX | NNXNNNXNN |
| XXXNNXNXN | XNXXXNXXN | XNNXNXXNX | NXXNXNXXX | NXNNNXXXX | NNNXXXXXN |
| XXXNNXNNX | XNXXXNXNX | XNNXNXXNN | NXXNXNXXN | NXNNNXXXN | NNNXXXXNX |
| XXXNNXNNN | XNXXXNXNN | XNNXNXNXX | NXXNXNXNX | NXNNNXXNX | NNNXXXXNN |
| XXXNNNXXN | XNXXXNNXX | XNNXNXNXN | NXXNXNXNN | NXNNNXXNN | NNNXXXNXX |
| XXXNNNXNX | XNXXXNNXN | XNNXNXNNX | NXXNXNNXX | NXNNNXNXX | NNNXXXNXN |
| XXXNNNXNN | XNXXXNNNX | XNNXNXNNN | NXXNXNNXN | NXNNNXNXN | NNNXXXNNX |
| XXNXXXXXN | XNXXNXXXX | XNNXNNXXX | NXXNXNNNX | NXNNNXNNX | NNNXXXNNN |
| XXNXXXXNX | XNXXNXXXN | XNNXNNXXN | NXXNNXXXX | NXNNNXNNN | NNNXXNXXX |
| XXNXXXXNN | XNXXNXXNX | XNNXNNXNX | NXXNNXXXN | NNXXXXXXN | NNNXXNXXN |
| XXNXXXNXX | XNXXNXXNN | XNNXNNXNN | NXXNNXXNX | NNXXXXXNX | NNNXXNXNX |
| XXNXXXNXN | XNXXNXNXX | XNNXNNNXX | NXXNNXXNN | NNXXXXXNN | NNNXXNXNN |
| XXNXXXNNX | XNXXNXNXN | XNNXNNNXN | NXXNNXNXX | NNXXXXNXX | NNNXXNNXX |
| XXNXXXNNN | XNXXNXNNX | XNNNXXXXN | NXXNNXNXN | NNXXXXNXN | NNNXXNNXN |
| XXNXXNXXX | XNXXNXNNN | XNNNXXXNX | NXXNNXNNX | NNXXXXNNX | NNNXXNNNX |
| XXNXXNXXN | XNXXNNXXX | XNNNXXXNN | NXXNNXNNN | NNXXXXNNN | NNNXNXXXX |
| XXNXXNXNX | XNXXNNXXN | XNNNXXNXX | NXXNNNXXX | NNXXXNXXX | NNNXNXXXN |
| XXNXXNXNN | XNXXNNXNX | XNNNXXNXN | NXXNNNXXN | NNXXXNXXN | NNNXNXXNX |
| XXNXXNNXX | XNXXNNXNN | XNNNXXNNX | NXXNNNXNX | NNXXXNXNX | NNNXNXXNN |
| XXNXXNNXN | XNXXNNNXX | XNNNXXNNN | NXXNNNXNN | NNXXXNXNN | NNNXNXNXX |
| XXNXXNNNX | XNXXNNNXN | XNNNXNXXX | NXNXXXXXX | NNXXXNNXX | NNNXNXNXN |
| XXNXNXXXX | XNXNXXXXX | XNNNXNXXN | NXNXXXXXN | NNXXXNNXN | NNNXNXNNX |
| XXNXNXXXN | XNXNXXXXN | XNNNXNXNX | NXNXXXXNX | NNXXXNNNX | NNNXNXNNN |
| XXNXNXXNX | XNXNXXXNX | XNNNXNXNN | NXNXXXXNN | NNXXNXXXX | NNNXNNXXX |
| XXNXNXXNN | XNXNXXXNN | XNNNXNNXX | NXNXXXNXX | NNXXNXXXN | NNNXNNXXN |
| XXNXNXNXX | XNXNXXNXX | XNNNXNNXN | NXNXXXNXN | NNXXNXXNX | NNNXNNXNX |
| XXNXNXNXN | XNXNXXNXN | XNNNXNNNX | NXNXXXNNX | NNXXNXXNN | NNNXNNXNN |
| XXNXNXNNX | XNXNXXNNX | XNNNXNNNN | NXNXXXNNN | NNXXNXNXX | NNNXNNNXX |
| XXNXNXNNN | XNXNXXNNN | NXXXXXXXN | NXNXXNXXX | NNXXNXNXN | NNNXNNNXN |

In yet another alternate embodiment, the plurality of oligonucleotides may comprise the formula NₘZ_{q}, wherein N and Z are nucleotides as defined above, m ranges from 2 to 13, q ranges from 1 to 12, the sum total of m and q is 14, and the at least two N residues are separated by at least one Z residue. In another embodiment, the plurality of oligonucleotides may comprise the formula NₘZ_{q}, wherein N and Z are nucleotides as defined above, m ranges from 2 to 12, q ranges from 1 to 11, the sum total of m and q is 13, and the at least two N residues are separated by at least one Z residue. In still another embodiment, the plurality of oligonucleotides may comprise the formula NₘZ_{q}, wherein N and Z are nucleotides as defined above, m ranges from 2 to 11, q ranges from 1 to 10, the sum total of m and q is 12, and the at least two N residues are separated by at least one Z residue. In another embodiment, the plurality of oligonucleotides may comprise the formula NₘZ_{q}, wherein N and Z are nucleotides as defined above, m ranges from 2 to 10, q ranges from 1 to 9, the sum total of m and q is 11, and the at least two N residues are separated by at least one Z residue. In yet another embodiment, the plurality of oligonucleotides may comprise the formula NₘZ_{q}, wherein N and Z are nucleotides as defined above, m ranges from 2 to 9, q ranges from 1 to 8, the sum total of m and q is 10, and the at least two N residues are separated by at least one Z residue. In still another embodiment, the plurality of oligonucleotides may comprise the formula NₘZ_{q}, wherein N and Z are nucleotides as defined above, m ranges from 2 to 7, q ranges from 1 to 6, the sum total of m and q is 8, and the at least two N residues are separated by at least one Z residue. In another embodiment, the plurality of oligonucleotides may comprise the formula NₘZ_{q}, wherein N and Z are nucleotides as defined above, m ranges from 2 to 6, q ranges from 1 to 5, the sum total of m and q is 7, and the at least two N residues are separated by at least one Z residue. In yet another embodiment, the plurality of oligonucleotides may comprise the formula NₘZ_{q}, wherein N and Z are nucleotides as defined above, m ranges from 2 to 5, q ranges from 1 to 4, the sum total of m and q is 6, and the at least two N residues are separated by at least one Z residue. In a preferred embodiment, the plurality of oligonucleotides may comprise the formula NₘZ_{q}, wherein N and Z are nucleotides as defined above, m ranges from 2 to 8, q ranges from 1 to 7, the sum total of m and q is 9, and the at least two N residues are separated by at least one Z residue. Table E presents (5′ to 3′) sequences of this preferred embodiment, i.e., a 9-nucleotide long semi-random region.

TABLE E
Nucleotide sequences (5′ to 3′) of an exemplary semi-random region.

| | | | | | |
|---|---|---|---|---|---|
| ZZZZZZNZN | ZZNNZZNNZ | ZNZNNNZNN | NZZZNZZZN | NZNZNNNNN | NNZNZNNNZ |
| ZZZZZNZZN | ZZNNZZNNN | ZNZNNNNZZ | NZZZNZZNZ | NZNNZZZZZ | NNZNZNNNN |
| ZZZZZNZNZ | ZZNNZNZZZ | ZNZNNNNZN | NZZZNZZNN | NZNNZZZZN | NNZNNZZZZ |
| ZZZZZNZNN | ZZNNZNZZN | ZNZNNNNNZ | NZZZNZNZZ | NZNNZZZNZ | NNZNNZZZN |
| ZZZZZNNZN | ZZNNZNZNZ | ZNZNNNNNN | NZZZNZNZN | NZNNZZZNN | NNZNNZZNZ |
| ZZZZNZZZN | ZZNNZNZNN | ZNNZZZZZN | NZZZNZNNZ | NZNNZZNZZ | NNZNNZZNN |
| ZZZZNZZNZ | ZZNNZNNZZ | ZNNZZZZNZ | NZZZNZNNN | NZNNZZNZN | NNZNNZNZZ |
| ZZZZNZZNN | ZZNNZNNZN | ZNNZZZZNN | NZZZNNZZZ | NZNNZZNNZ | NNZNNZNZN |
| ZZZZNZNZZ | ZZNNZNNNZ | ZNNZZZNZZ | NZZZNNZZN | NZNNZZNNN | NNZNNZNNZ |
| ZZZZNZNZN | ZZNNZNNNN | ZNNZZZNZN | NZZZNNZNZ | NZNNZNZZZ | NNZNNZNNN |
| ZZZZNZNNZ | ZZNNNZZZN | ZNNZZZNNZ | NZZZNNZNN | NZNNZNZZN | NNZNNNZZZ |
| ZZZZNZNNN | ZZNNNZZNZ | ZNNZZZNNN | NZZZNNNZZ | NZNNZNZNZ | NNZNNNZZN |
| ZZZZNNZZN | ZZNNNZZNN | ZNNZZNZZZ | NZZZNNNZN | NZNNZNZNN | NNZNNNZNZ |
| ZZZZNNZNZ | ZZNNNZNZZ | ZNNZZNZZN | NZZZNNNNZ | NZNNZNNZZ | NNZNNNZNN |
| ZZZZNNZNN | ZZNNNZNZN | ZNNZZNZNZ | NZZZNNNNN | NZNNZNNZN | NNZNNNNZZ |
| ZZZZNNNZN | ZZNNNZNNZ | ZNNZZNZNN | NZZNZZZZZ | NZNNZNNNZ | NNZNNNNZN |
| ZZZNZZZZZ | ZZNNNZNNN | ZNNZZNNZZ | NZZNZZZZN | NZNNZNNNN | NNZNNNNNZ |
| ZZZNZZZZN | ZZNNNNZZN | ZNNZZNNZN | NZZNZZZNZ | NZNNNZZZZ | NNZNNNNNN |
| ZZZNZZZNZ | ZZNNNNZNZ | ZNNZZNNNZ | NZZNZZZNN | NZNNNZZZN | NNNZZZZZN |
| ZZZNZZZNN | ZZNNNNZNN | ZNNZZNNNN | NZZNZZNZZ | NZNNNZZNZ | NNNZZZZNZ |
| ZZZNZZNZZ | ZZNNNNNZN | ZNNZNZZZZ | NZZNZZNZN | NZNNNZZNN | NNNZZZZNN |
| ZZZNZZNZN | ZNZZZZZZN | ZNNZNZZZN | NZZNZZNNZ | NZNNNZNZZ | NNNZZZNZZ |
| ZZZNZZNNZ | ZNZZZZZNZ | ZNNZNZZNZ | NZZNZZNNN | NZNNNZNZN | NNNZZZNZN |
| ZZZNZZNNN | ZNZZZZZNN | ZNNZNZZNN | NZZNZNZZZ | NZNNNZNNZ | NNNZZZNNZ |
| ZZZNZNZZZ | ZNZZZZNZZ | ZNNZNZNZZ | NZZNZNZZN | NZNNNZNNN | NNNZZZNNN |
| ZZZNZNZZN | ZNZZZZNZN | ZNNZNZNZN | NZZNZNZNZ | NZNNNNZZZ | NNNZZNZZZ |
| ZZZNZNZNZ | ZNZZZZNNZ | ZNNZNZNNZ | NZZNZNZNN | NZNNNNZZN | NNNZZNZZN |
| ZZZNZNZNN | ZNZZZZNNN | ZNNZNZNNN | NZZNZNNZZ | NZNNNNZNZ | NNNZZNZNZ |
| ZZZNZNNZZ | ZNZZZNZZZ | ZNNZNNZZZ | NZZNZNNZN | NZNNNNZNN | NNNZZNZNN |
| ZZZNZNNZN | ZNZZZNZZN | ZNNZNNZZN | NZZNZNNNZ | NZNNNNNZZ | NNNZZNNZZ |
| ZZZNZNNNZ | ZNZZZNZNZ | ZNNZNNZNZ | NZZNZNNNN | NZNNNNNZN | NNNZZNNZN |
| ZZZNZNNNN | ZNZZZNZNN | ZNNZNNZNN | NZZNNZZZZ | NZNNNNNNZ | NNNZZNNNZ |
| ZZZNNZZZN | ZNZZZNNZZ | ZNNZNNNZZ | NZZNNZZZN | NZNNNNNNN | NNNZZNNNN |
| ZZZNNZZNZ | ZNZZZNNZN | ZNNZNNNZN | NZZNNZZNZ | NNZZZZZZN | NNNZNZZZZ |
| ZZZNNZZNN | ZNZZZNNNZ | ZNNZNNNNZ | NZZNNZZNN | NNZZZZZNZ | NNNZNZZZN |
| ZZZNNZNZZ | ZNZZZNNNN | ZNNZNNNNN | NZZNNZNZZ | NNZZZZZNN | NNNZNZZNZ |
| ZZZNNZNZN | ZNZZNZZZZ | ZNNNZZZZN | NZZNNZNZN | NNZZZZNZZ | NNNZNZZNN |
| ZZZNNZNNZ | ZNZZNZZZN | ZNNNZZZNZ | NZZNNZNNZ | NNZZZZNZN | NNNZNZNZZ |
| ZZZNNZNNN | ZNZZNZZNZ | ZNNNZZZNN | NZZNNZNNN | NNZZZZNNZ | NNNZNZNZN |
| ZZZNNNZZN | ZNZZNZZNN | ZNNNZZNZZ | NZZNNNZZZ | NNZZZZNNN | NNNZNZNNZ |
| ZZZNNNZNZ | ZNZZNZNZZ | ZNNNZZNZN | NZZNNNZZN | NNZZZNZZZ | NNNZNZNNN |
| ZZZNNNZNN | ZNZZNZNZN | ZNNNZZNNZ | NZZNNNZNZ | NNZZZNZZN | NNNZNNZZZ |
| ZZZNNNNZN | ZNZZNZNNZ | ZNNNZZNNN | NZZNNNZNN | NNZZZNZNZ | NNNZNNZZN |
| ZZNZZZZZN | ZNZZNZNNN | ZNNNZNZZZ | NZZNNNNZZ | NNZZZNZNN | NNNZNNZNZ |
| ZZNZZZZNZ | ZNZZNNZZZ | ZNNNZNZZN | NZZNNNNZN | NNZZZNNZZ | NNNZNNZNN |
| ZZNZZZZNN | ZNZZNNZZN | ZNNNZNZNZ | NZZNNNNNZ | NNZZZNNZN | NNNZNNNZZ |
| ZZNZZZNZZ | ZNZZNNZNZ | ZNNNZNZNN | NZZNNNNNN | NNZZZNNNZ | NNNZNNNZN |
| ZZNZZZNZN | ZNZZNNZNN | ZNNNZNNZZ | NZNZZZZZZ | NNZZZNNNN | NNNZNNNNZ |
| ZZNZZZNNZ | ZNZZNNNZZ | ZNNNZNNZN | NZNZZZZZN | NNZZNZZZZ | NNNZNNNNN |
| ZZNZZZNNN | ZNZZNNNZN | ZNNNZNNNZ | NZNZZZZNZ | NNZZNZZZN | NNNNZZZZZ |
| ZZNZZNZZZ | ZNZZNNNNZ | ZNNNZNNNN | NZNZZZZNN | NNZZNZZNZ | NNNNZZZZN |
| ZZNZZNZZN | ZNZZNNNNN | ZNNNNZZZN | NZNZZZNZZ | NNZZNZZNN | NNNNZZZNZ |
| ZZNZZNZNZ | ZNZNZZZZZ | ZNNNNZZNZ | NZNZZZNZN | NNZZNZNZZ | NNNNZZZNN |
| ZZNZZNZNN | ZNZNZZZZN | ZNNNNZZNN | NZNZZZNNZ | NNZZNZNZN | NNNNZZNZZ |
| ZZNZZNNZZ | ZNZNZZZNZ | ZNNNNZNZZ | NZNZZZNNN | NNZZNZNNZ | NNNNZZNZN |
| ZZNZZNNZN | ZNZNZZZNN | ZNNNNZNZN | NZNZZNZZZ | NNZZNZNNN | NNNNZZNNZ |
| ZZNZZNNNZ | ZNZNZZNZZ | ZNNNNZNNZ | NZNZZNZZN | NNZZNNZZZ | NNNNZZNNN |
| ZZNZZNNNN | ZNZNZZNZN | ZNNNNZNNN | NZNZZNZNZ | NNZZNNZZN | NNNNZNZZZ |
| ZZNZNZZZZ | ZNZNZZNNZ | ZNNNNNZZN | NZNZZNZNN | NNZZNNZNZ | NNNNZNZZN |
| ZZNZNZZZN | ZNZNZZNNN | ZNNNNNZNZ | NZNZZNNZZ | NNZZNNZNN | NNNNZNZNZ |
| ZZNZNZZNZ | ZNZNZNZZZ | ZNNNNNZNN | NZNZZNNZN | NNZZNNNZZ | NNNNZNZNN |
| ZZNZNZZNN | ZNZNZNZZN | ZNNNNNNZN | NZNZZNNNZ | NNZZNNNZN | NNNNZNNZZ |
| ZZNZNZNZZ | ZNZNZNZNZ | NZZZZZZZN | NZNZZNNNN | NNZZNNNNZ | NNNNZNNZN |
| ZZNZNZNZN | ZNZNZNZNN | NZZZZZZNZ | NZNZNZZZZ | NNZZNNNNN | NNNNZNNNZ |
| ZZNZNZNNZ | ZNZNZNNZZ | NZZZZZZNN | NZNZNZZZN | NNZNZZZZZ | NNNNZNNNN |
| ZZNZNZNNN | ZNZNZNNZN | NZZZZZNZZ | NZNZNZZNZ | NNZNZZZZN | NNNNNZZZZ |
| ZZNZNNZZZ | ZNZNZNNNZ | NZZZZZNZN | NZNZNZZNN | NNZNZZZNZ | NNNNNZZZN |
| ZZNZNNZZN | ZNZNZNNNN | NZZZZZNNZ | NZNZNZNZZ | NNZNZZZNN | NNNNNZZNZ |
| ZZNZNNZNZ | ZNZNNZZZZ | NZZZZZNNN | NZNZNZNZN | NNZNZZNZZ | NNNNNZZNN |
| ZZNZNNZNN | ZNZNNZZZN | NZZZZNZZZ | NZNZNZNNZ | NNZNZZNZN | NNNNNZNZZ |
| ZZNZNNNZZ | ZNZNNZZNZ | NZZZZNZZN | NZNZNZNNN | NNZNZZNNZ | NNNNNZNZN |
| ZZNZNNNZN | ZNZNNZZNN | NZZZZNZNZ | NZNZNNZZZ | NNZNZZNNN | NNNNNZNNZ |
| ZZNZNNNNZ | ZNZNNZNZZ | NZZZZNZNN | NZNZNNZZN | NNZNZNZZZ | NNNNNZNNN |
| ZZNZNNNNN | ZNZNNZNZN | NZZZZNNZZ | NZNZNNZNZ | NNZNZNZZN | NNNNNNZZZ |
| ZZNNZZZZN | ZNZNNZNNZ | NZZZZNNZN | NZNZNNZNN | NNZNZNZNZ | NNNNNNZZN |
| ZZNNZZZNZ | ZNZNNZNNN | NZZZZNNNZ | NZNZNNNZZ | NNZNZNZNN | NNNNNNZNZ |
| ZZNNZZZNN | ZNZNNNZZZ | NZZZZNNNN | NZNZNNNZN | NNZNZNNZZ | NNNNNNZNN |
| ZZNNZZNZZ | ZNZNNNZZN | NZZZNZZZZ | NZNZNNNNZ | NNZNZNNZN | NNNNNNNZN |
| ZZNNZZNZN | ZNZNNNZNZ | | | | |

In another alternate embodiment, the plurality of oligonucleotides may comprise formula NₘZ_{q}, wherein N and Z are nucleotides as defined above, m ranges from 2 to 13, q ranges from 1 to 12, the sum total of m and q ranges from 6 to 14, the at least two N residues are separated by at least one Z residue, and there are no more than three consecutive N residues. In this embodiment, therefore, non-complementary 2-fold degenerate nucleotides are interspersed throughout the sequence such that there are no long runs (≥4) of the complementary 4-fold degenerate nucleotide (N). In general, such a design may reduce self-hybridization and/or cross-hybridization within the plurality of oligonucleotides. In an exemplary embodiment, the plurality of oligonucleotides may comprise formula NₘZ_{q}, wherein N and Z are nucleotides as defined above, m ranges from 2 to 8, q ranges from 1 to 7, the sum total of m and q is 9, the at least two N residues are separated by at least one Z residue, and there are no more than three consecutive N residues. Table F lists the (5′ to 3′) sequences of this preferred embodiment, i.e., a 9-nucleotide long semi-random region containing no more that three consecutive N residues.

TABLE F
Nucleotide sequences (5′ to 3′) of an exemplary semi-random region having
no more than 3 consecutive N residues.

| | | | | | |
|---|---|---|---|---|---|
| ZZZZZZNZN | ZZNZNNZZZ | ZNZNZNZZZ | NZZZZZZNZ | NZNZZNZZN | NNZZNZNNZ |
| ZZZZZNZZN | ZZNZNNZZN | ZNZNZNZZN | NZZZZZZNN | NZNZZNZNZ | NNZZNZNNN |
| ZZZZZNZNZ | ZZNZNNZNZ | ZNZNZNZNZ | NZZZZZNZZ | NZNZZNZNN | NNZZNNZZZ |
| ZZZZZNZNN | ZZNZNNZNN | ZNZNZNZNN | NZZZZZNZN | NZNZZNNZZ | NNZZNNZZN |
| ZZZZZNNZN | ZZNZNNNZZ | ZNZNZNNZZ | NZZZZZNNZ | NZNZZNNZN | NNZZNNZNZ |
| ZZZZNZZZN | ZZNZNNNZN | ZNZNZNNZN | NZZZZZNNN | NZNZZNNNZ | NNZZNNZNN |
| ZZZZNZZNZ | ZZNNZZZZN | ZNZNZNNNZ | NZZZZNZZZ | NZNZNZZZZ | NNZZNNNZZ |
| ZZZZNZZNN | ZZNNZZZNZ | ZNZNNZZZZ | NZZZZNZZN | NZNZNZZZN | NNZZNNNZN |
| ZZZZNZNZZ | ZZNNZZZNN | ZNZNNZZZN | NZZZZNZNZ | NZNZNZZNZ | NNZNZZZZZ |
| ZZZZNZNZN | ZZNNZZNZZ | ZNZNNZZNZ | NZZZZNZNN | NZNZNZZNN | NNZNZZZZN |
| ZZZZNZNNZ | ZZNNZZNZN | ZNZNNZZNN | NZZZZNNZZ | NZNZNZNZZ | NNZNZZZNZ |
| ZZZZNZNNN | ZZNNZZNNZ | ZNZNNZNZZ | NZZZZNNZN | NZNZNZNZN | NNZNZZZNN |
| ZZZZNNZZN | ZZNNZZNNN | ZNZNNZNZN | NZZZZNNNZ | NZNZNZNNZ | NNZNZZNZZ |
| ZZZZNNZNZ | ZZNNZNZZZ | ZNZNNZNNZ | NZZZNZZZZ | NZNZNZNNN | NNZNZZNZN |
| ZZZZNNZNN | ZZNNZNZZN | ZNZNNZNNN | NZZZNZZZN | NZNZNNZZZ | NNZNZZNNZ |
| ZZZZNNNZN | ZZNNZNZNZ | ZNZNNNZZZ | NZZZNZZNZ | NZNZNNZZN | NNZNZZNNN |
| ZZZNZZZZZ | ZZNNZNZNN | ZNZNNNZZN | NZZZNZZNN | NZNZNNZNZ | NNZNZNZZZ |
| ZZZNZZZZN | ZZNNZNNZZ | ZNZNNNZNZ | NZZZNZNZZ | NZNZNNZNN | NNZNZNZZN |
| ZZZNZZZNZ | ZZNNZNNZN | ZNZNNNZNN | NZZZNZNZN | NZNZNNNZZ | NNZNZNZNZ |
| ZZZNZZZNN | ZZNNZNNNZ | ZNNZZZZZN | NZZZNZNNZ | NZNZNNNZN | NNZNZNZNN |
| ZZZNZZNZZ | ZZNNNZZZN | ZNNZZZZNZ | NZZZNZNNN | NZNNZZZZZ | NNZNZNNZZ |
| ZZZNZZNZN | ZZNNNZZNZ | ZNNZZZZNN | NZZZNNZZZ | NZNNZZZZN | NNZNZNNZN |
| ZZZNZZNNZ | ZZNNNZZNN | ZNNZZZNZZ | NZZZNNZZN | NZNNZZZNZ | NNZNZNNNZ |
| ZZZNZZNNN | ZZNNNZNZZ | ZNNZZZNZN | NZZZNNZNZ | NZNNZZZNN | NNZNNZZZZ |
| ZZZNZNZZZ | ZZNNNZNZN | ZNNZZZNNZ | NZZZNNZNN | NZNNZZNZZ | NNZNNZZZN |
| ZZZNZNZZN | ZZNNNZNNZ | ZNNZZZNNN | NZZZNNNZZ | NZNNZZNZN | NNZNNZZNZ |
| ZZZNZNZNZ | ZZNNNZNNN | ZNNZZNZZZ | NZZZNNNZN | NZNNZZNNZ | NNZNNZZNN |
| ZZZNZNZNN | ZNZZZZZZN | ZNNZZNZZN | NZZNZZZZZ | NZNNZZNNN | NNZNNZNZZ |
| ZZZNZNNZZ | ZNZZZZZNZ | ZNNZZNZNZ | NZZNZZZZN | NZNNZNZZZ | NNZNNZNZN |
| ZZZNZNNZN | ZNZZZZZNN | ZNNZZNZNN | NZZNZZZNZ | NZNNZNZZN | NNZNNZNNZ |
| ZZZNZNNNZ | ZNZZZZNZZ | ZNNZZNNZZ | NZZNZZZNN | NZNNZNZNZ | NNZNNZNNN |
| ZZZNNZZZN | ZNZZZZNZN | ZNNZZNNZN | NZZNZZNZZ | NZNNZNZNN | NNZNNNZZZ |
| ZZZNNZZNZ | ZNZZZZNNZ | ZNNZZNNNZ | NZZNZZNZN | NZNNZNNZZ | NNZNNNZZN |
| ZZZNNZZNN | ZNZZZZNNN | ZNNZNZZZZ | NZZNZZNNZ | NZNNZNNZN | NNZNNNZNZ |
| ZZZNNZNZZ | ZNZZZNZZZ | ZNNZNZZZN | NZZNZZNNN | NZNNZNNNZ | NNZNNNZNN |
| ZZZNNZNZN | ZNZZZNZZN | ZNNZNZZNZ | NZZNZNZZZ | NZNNNZZZZ | NNNZZZZZN |
| ZZZNNZNNZ | ZNZZZNZNZ | ZNNZNZZNN | NZZNZNZZN | NZNNNZZZN | NNNZZZZNZ |
| ZZZNNZNNN | ZNZZZNZNN | ZNNZNZNZZ | NZZNZNZNZ | NZNNNZZNZ | NNNZZZZNN |
| ZZZNNNZZN | ZNZZZNNZZ | ZNNZNZNZN | NZZNZNZNN | NZNNNZZNN | NNNZZZNZZ |
| ZZZNNNZNZ | ZNZZZNNZN | ZNNZNZNNZ | NZZNZNNZZ | NZNNNZNZZ | NNNZZZNZN |
| ZZZNNNZNN | ZNZZZNNNZ | ZNNZNZNNN | NZZNZNNZN | NZNNNZNZN | NNNZZZNNZ |
| ZZNZZZZZN | ZNZZNZZZZ | ZNNZNNZZZ | NZZNZNNNZ | NZNNNZNNZ | NNNZZZNNN |
| ZZNZZZZNZ | ZNZZNZZZN | ZNNZNNZZN | NZZNNZZZZ | NZNNNZNNN | NNNZZNZZZ |
| ZZNZZZZNN | ZNZZNZZNZ | ZNNZNNZNZ | NZZNNZZZN | NNZZZZZZN | NNNZZNZZN |
| ZZNZZZNZZ | ZNZZNZZNN | ZNNZNNZNN | NZZNNZZNZ | NNZZZZZNZ | NNNZZNZNZ |
| ZZNZZZNZN | ZNZZNZNZZ | ZNNZNNNZZ | NZZNNZZNN | NNZZZZZNN | NNNZZNZNN |
| ZZNZZZNNZ | ZNZZNZNZN | ZNNZNNNZN | NZZNNZNZZ | NNZZZZNZZ | NNNZZNNZZ |
| ZZNZZZNNN | ZNZZNZNNZ | ZNNNZZZZN | NZZNNZNZN | NNZZZZNZN | NNNZZNNZN |
| ZZNZZNZZZ | ZNZZNZNNN | ZNNNZZZNZ | NZZNNZNNZ | NNZZZZNNZ | NNNZZNNNZ |
| ZZNZZNZZN | ZNZZNNZZZ | ZNNNZZZNN | NZZNNZNNN | NNZZZZNNN | NNNZNZZZZ |
| ZZNZZNZNZ | ZNZZNNZZN | ZNNNZZNZZ | NZZNNNZZZ | NNZZZNZZZ | NNNZNZZZN |
| ZZNZZNZNN | ZNZZNNZNZ | ZNNNZZNZN | NZZNNNZZN | NNZZZNZZN | NNNZNZZNZ |
| ZZNZZNNZZ | ZNZZNNZNN | ZNNNZZNNZ | NZZNNNZNZ | NNZZZNZNZ | NNNZNZZNN |
| ZZNZZNNZN | ZNZZNNNZZ | ZNNNZZNNN | NZZNNNZNN | NNZZZNZNN | NNNZNZNZZ |
| ZZNZZNNNZ | ZNZZNNNZN | ZNNNZNZZZ | NZNZZZZZZ | NNZZZNNZZ | NNNZNZNZN |
| ZZNZNZZZZ | ZNZNZZZZZ | ZNNNZNZZN | NZNZZZZZN | NNZZZNNZN | NNNZNZNNZ |
| ZZNZNZZZN | ZNZNZZZZN | ZNNNZNZNZ | NZNZZZZNZ | NNZZZNNNZ | NNNZNZNNN |
| ZZNZNZZNZ | ZNZNZZZNZ | ZNNNZNZNN | NZNZZZZNN | NNZZNZZZZ | NNNZNNZZZ |
| ZZNZNZZNN | ZNZNZZZNN | ZNNNZNNZZ | NZNZZZNZZ | NNZZNZZZN | NNNZNNZZN |
| ZZNZNZNZZ | ZNZNZZNZZ | ZNNNZNNZN | NZNZZZNZN | NNZZNZZNZ | NNNZNNZNZ |
| ZZNZNZNZN | ZNZNZZNZN | ZNNNZNNNZ | NZNZZZNNZ | NNZZNZZNN | NNNZNNZNN |
| ZZNZNZNNZ | ZNZNZZNNZ | ZNNNZNNNN | NZNZZZNNN | NNZZNZNZZ | NNNZNNNZZ |
| ZZNZNZNNN | ZNZNZZNNN | NZZZZZZZN | NZNZZNZZZ | NNZZNZNZN | NNNZNNNZN |

In another alternate embodiment, the plurality of oligonucleotides may comprise the formula XₚZ_{q}, wherein X and Z are nucleotides as defined above, p and q range from 1 to 13, and the sum total of p and q is 14. In another embodiment, the plurality of oligonucleotides may comprise the formula XₚZ_{q}, wherein X and Z are nucleotides as defined above, p and q range from 1 to 12, and the sum total of p and q is 13. In yet another embodiment, the plurality of oligonucleotides may comprise the formula XₚZ_{q}, wherein X and Z are nucleotides as defined above, p and q range from 1 to 11, and the sum total of p and q is 12. In still another embodiment, the plurality of oligonucleotides may comprise the formula XₚZ_{q}, wherein X and Z are nucleotides as defined above, p and q range from 1 to 10, and the sum total of p and q is 11. In another embodiment, the plurality of oligonucleotides may comprise the formula XₚZ_{q}, wherein X and Z are nucleotides as defined above, p and q range from 1 to 9, and the sum total of p and q is 10. In still another alternate embodiment, the plurality of oligonucleotides may comprise the formula XₚZ_{q}, wherein X and Z are nucleotides as defined above, p and q range from 1 to 8, and the sum total of p and q is 9. In still another embodiment, the plurality of oligonucleotides may comprise the formula XₚZ_{q}, wherein X and Z are nucleotides as defined above, p and q range from 1 to 7, and the sum total of p and q is 8. In yet another embodiment, the plurality of oligonucleotides may comprise the formula XₚZ_{q}, wherein X and Z are nucleotides as defined above, p and q range from 1 to 6, and the sum total of p and q is 7. In a further embodiment, the plurality of oligonucleotides may comprise the formula XₚZ_{q}, wherein X and Z are nucleotides as defined above, p and q range from 1 to 5, and the sum total of p and q is 6.

In still other embodiments, in which both m and q are 0, the plurality of oligonucleotides comprises the formula Xₚ, wherein X is a 3-fold degenerate nucleotide and p is an integer from 2 to 20. The plurality of oligonucleotides, therefore, may comprise the following formulas: B2-20(SEQ ID NO:49), D₂₋₂₀(SEQ ID NO: 50), H2-20 (SEQ ID NO: 51), or V₂₋₂₀ (SEQ ID NO: 52). The plurality of oligonucleotides having these formulas may range from about 2 nucleotides to about 8 nucleotides in length, from about 8 nucleotides to about 14 nucleotides in length, or from about 14 nucleotides to about 20 nucleotides in length. In a preferred embodiment, the plurality of oligonucleotides may be about 9 nucleotides in length.

### (b) Optional Non-random Sequence

The oligonucleotides described above may further comprise a non-random sequence comprising standard (non-degenerate) nucleotides. The non-random sequence is located at the 5′ end of each oligonucleotide. In general, the sequence of non-degenerate nucleotides is constant among the oligonucleotides of a plurality. The constant non-degenerate sequence typically comprises a known sequence, such as a universal priming site. Non-limiting examples of suitable universal priming sites include T7 promoter sequence, T3 promoter sequence, SP6 promoter sequence, M13 forward sequence, or M13 reverse sequence. Alternatively the constant non-degenerate sequence may comprise essentially any artificial sequence that is not present in the nucleic acid that is to be amplified. In one embodiment, the constant non-degenerate sequence may comprise the sequence 5′-GTAGGTTGAGGATAGGAGGGTTAGG-3′ (SEQ ID NO:3). In another embodiment, the constant non-degenerate sequence may comprise the sequence 5′-GTGGTGTGTTGGGTGTGTTTGG-3′ (SEQ ID NO:28).

The constant non-degenerate sequence may range from about 6 nucleotides to about 100 nucleotides in length. In one embodiment, the constant, non-degenerate sequence may range from about 10 nucleotides to about 40 nucleotides in length. In another embodiment, the constant non-degenerate sequence may range from about 14 nucleotides to about 30 nucleotides in length. In yet another embodiment, the constant non-degenerate sequence may range from about 18 nucleotides to about 26 nucleotides in length. In still another embodiment, the constant non-degenerate sequence may range from about 22 nucleotides to about 25 nucleotides in length.

In some embodiments, additional nucleotides may be added to the 5′ end of the constant non-degenerate sequence of each oligonucleotide of the plurality. For example, nucleotides may be added to increase the melting temperature of the plurality of oligonucleotides. The additional nucleotides may comprise G residues, C residues, or a combination thereof. The number of additional nucleotides may range from about 1 nucleotide to about 10 nucleotides, preferably from about 3 nucleotides to about 6 nucleotides, and more preferably about 4 nucleotides.

### (II) Method for Amplifying a Population of Target Nucleic Acids

Another aspect of the invention provides a method for amplifying a population of target nucleic acids by creating a library of amplifiable molecules, which then may be further amplified. The library of amplifiable molecules is generated in a sequence independent manner by using the plurality of degenerate oligonucleotide primers of the invention to provide a plurality of replication initiation sites throughout the target nucleic acid. The semi-random sequence of the degenerate oligonucleotide primers minimizes intramolecular and intermolecular interactions among the plurality of oligonucleotide primers while still providing sequence diversity, thereby facilitating replication of the entire target nucleic acid. Thus, the target nucleic acid may be amplified without compromising the representation of any given sequence and without significant bias (i.e., 3′ end bias). The amplified target nucleic acid may be a whole genome or a whole transcriptome.

### (a) Creating a Library

A library of amplifiable molecules representative of the population of target nucleic acids may be generated by contacting the target nucleic acids with a plurality of degenerate oligonucleotide primers of the invention. The degenerate oligonucleotide primers hybridize at random sites scattered somewhat equally throughout the target nucleic acid to provide a plurality of priming sites for replication of the target nucleic acid. The target nucleic acid may be replicated by an enzyme with strand-displacing activity, such that replicated strands are displaced during replication and serve as templates for additional rounds of replication. Alternatively, the target nucleic acid may be replicated via a two-step process, i.e., first strand cDNA is synthesized with a reverse transcriptase and second strand cDNA is synthesized with an enzyme without strand-displacing activity. As a consequence of either method, the amount of replicated strands exceeds the amount of starting target nucleic acids, indicating amplification of the target nucleic acid.

### (i) Target Nucleic Acid

The population of target nucleic acids can and will vary. In one embodiment, the population of target nucleic acids may be genomic DNA. Genomic DNA refers to one or more chromosomal DNA molecules occurring naturally in the nucleus or an organelle (e.g., mitochondrion, chloroplast, or kinetoplast) of a eukaryotic cell, a eubacterial cell, an archaeal cell, or a virus. These molecules contain sequences that are transcribed into RNA, as well as sequences that are not transcribed into RNA. As such, genomic DNA may comprise the whole genome of an organism or it may comprise a portion of the genome, such as a single chromosome or a fragment thereof.

In another embodiment, the population of target nucleic acids may be a population of RNA molecules. The RNA molecules may be messenger RNA molecules or small RNA molecules. The population of RNA molecules may comprise a transcriptome, which is defined as the set of all RNA molecules expressed in one cell or a population of cells. The set of RNA molecules may include messenger RNAs and/or microRNAs and other small RNAs. The term, transcriptome, may refer to the total set of RNA molecules in a given organism or the specific subset of RNA molecules present in a particular cell type.

The population of target nucleic acids may be derived from eukaryotes, eubacteria, archaea, or viruses. Non-limiting examples of suitable eukaryotes include humans, mice, mammals, vertebrates, invertebrates, plants, fungi, yeast, and protozoa. In a preferred embodiment, the population of nucleic acids is derived from a human. Non-limiting sources of target nucleic acids include a genomic DNA preparation, a total RNA preparation, a poly(A)⁺ RNA preparation, a poly(A)⁻ RNA preparation, a small RNA preparation, a single cell, a cell lysate, cultured cells, a tissue sample, a fixed tissue, a frozen tissue, an embedded tissue, a biopsied tissue, a tissue swab, or a biological fluid. Suitable body fluids include, but are not limited to, whole blood, buffy coats, serum, saliva, cerebrospinal fluid, pleural fluid, lymphatic fluid, milk, sputum, semen, and urine.

In some embodiments, the target nucleic acid may be randomly fragmented prior to contact with the plurality of oligonucleotide primers. The target nucleic acid may be randomly fragmented by mechanical means, such as physically shearing the nucleic acid by passing it through a narrow capillary or orifice, sonicating the nucleic acid, and/or nebulizing the nucleic acid. Alternatively, the nucleic acid may be randomly fragmented by chemical means, such as acid hydrolysis, alkaline hydrolysis, formalin fixation, hydrolysis by metal complexes (e.g., porphyrins), and/or hydrolysis by hydroxyl radicals. The target nucleic acid may also be randomly fragmented by thermal means, such as heating the nucleic acid in a solution of low ionic strength and neutral pH. The temperature may range from about 90° C. to about 100° C., and preferably about 95° C. The solution of low ionic strength may comprise from about 10 mM to about 20 mM of Tris-HCl and from about 0.1 mM to about 1 mM of EDTA, with a pH of about 7.5 to about 8.5. The duration of the heating period may range from about 1 minute to about 10 minutes. Alternatively, the nucleic acid may be fragmented by enzymatic means, such as partial digestion with DNase I or an RNase. Alternatively, DNA may be fragmented by digestion with a restriction endonuclease that recognizes multiple tetra-nucleotide recognition sequences (e.g., CviJI) in the presence of a divalent cation. Depending upon the method used to fragment the nucleic acid, the size of the fragments may range from about 100 base pairs to about 5000 base pairs, or from about 50 nucleotides to about 2500 nucleotides.

The amount of nucleic acid available as target can and will vary depending upon the type and quality of the nucleic acid. In general, the amount of target nucleic acid may range from about 0.1 picograms (pg) to about 1,000 nanograms (ng). In embodiments in which the target nucleic acid is genomic DNA, the amount of target DNA may be about 1 ng for simple genomes such as those from bacteria, about 10 ng for a complex genome such as that of human, about 5 pg for a single human cell, or about 200 ng for partially degraded DNA extracted from fixed tissue. In embodiments in which the target nucleic acid is high quality total RNA, the amount of target RNA may range from about 0.1 pg to about 50 ng, or more preferably from about 10 pg to about 500 pg. In other embodiments in which the target nucleic acid is partially degraded total RNA, the amount of target RNA may range from about 25 ng to about 1,000 ng. For embodiments in which the target nucleic acid is RNA from a single cell, one skilled in the art will appreciate that the amount of RNA in a cell varies among different cell types.

### (ii) Plurality of Oligonucleotide Primers

The plurality of oligonucleotide primers that is contacted with the target nucleic acid was described above in section (I)(a). The oligonucleotide primers comprise a semi-random region comprising a mixture of fully (i.e., 4-fold) degenerate and partially (i.e., 3-fold and/or 2-fold) degenerate nucleotides. The partially degenerate nucleotides are dispersed among the fully degenerate nucleotides such at least one 2-fold or 3-fold degenerate nucleotide separates the at least two 4-fold degenerate nucleotides. The presence of non-complementary 2-fold degenerate nucleotides and/or partially non-complementary 3-fold degenerate nucleotides reduces the ability of the oligonucleotide primers comprising fully degenerate nucleotides to self-hybridize and/or cross-hybridize (and form primer-dimers), while still providing high sequence diversity.

In a preferred embodiment, the plurality of oligonucleotide primers used in the method of the invention comprise the formula NₘXₚ, NₘZ_{q}, or a combination thereof, wherein N, X, and Z are degenerate nucleotides as defined above, m is from 2 to 13, p and q are each from 1 to 12, and the sum total of the two integers is from 6 to 14, and the at least two N residues are separated by at least one X or Z residue. In another preferred embodiment, the plurality of oligonucleotide primers used in the method comprise the formula NₘXₚ, NₘZ_{q}, or a combination thereof, wherein N, X, and Z are degenerate nucleotides as defined above, m is an integer from 2 to 8, p and q are integers from 1 to 7, the sum total of the two integers is 9, the at least two N residues are separated by at least one X or Z residue, and there are no more than three consecutive N residues (see Tables D and F). In preferred embodiments, X is D and Y is K. In an especially preferred embodiment, the plurality of oligonucleotide primers used in the method of the invention have the following (5′-3′) sequences: KNNNKNKNK, NKNNKNNKK, and NNNKNKKNK. The preferred oligonucleotide primers may further comprise a constant non-degenerate sequence at the 5′ end of each oligonucleotide, as described above in section (I)(b).

The plurality of oligonucleotide primers contacted with the target nucleic acid may have a single sequence. For example, the (5′-3′) sequence of the plurality of degenerate oligonucleotide primers may be XNNNXNXNX. The degeneracy of this oligonucleotide primer may be calculated using the formula presented above (i.e., degeneracy=82,944=3⁴×4⁵). Alternatively, the plurality of oligonucleotide primers contacted with the target nucleic acid may be a mixture of degenerate oligonucleotide primers having different sequences. The mixture may comprise two degenerate oligonucleotide primers, three degenerate oligonucleotide primers, four degenerate oligonucleotide primers, etc. As an example, the mixture may comprise three degenerate oligonucleotide primers having the following (5′-3′) sequences: XNNNXNXNX, NNNXNXXNX, XXXNNXXNX. In this example, the degeneracy of the mixture of oligonucleotide primers is 212,544[=(3⁴×4⁵)+(3⁴×4⁵)+(3⁶33 4³)]. The mixture may comprise degenerate oligonucleotide primers comprising 3-fold degenerate nucleotides and/or 2-fold degenerate nucleotides (i.e., formulas NₘXₚ and/or NₘZ_{q}).

Because of the large number of sequences represented in the plurality of degenerate oligonucleotide primers of the invention, a subset of oligonucleotide primers will generally have many complementary sequences dispersed throughout the population of target nucleic acids. Accordingly, the subset of complementary oligonucleotide primers will hybridize with the target nucleic acid, thereby forming a plurality of nucleic acid-primer duplexes and providing a plurality of priming sites for nucleic acid replication.

In some embodiments, in addition to the plurality of oligonucleotide primers, an oligo dT or anchor oligo dT primer may also be contacted with the population of target nucleic acids. The anchor oligo dT primer may comprise (5′ to 3′) a string of deoxythymidylic acid (dT) residues followed by two additional ribonucleotides represented by VN, wherein V is either G, C, or A and N is either G, C, A, or U. The VN ribonucleotide anchor allows the primer to hybridize only at the 5′ end of the poly(A) tail of a target messenger RNA, such that the messenger RNA may be reverse transcribed into cDNA. One skilled in the art will appreciate that an oligo dT primer may comprise other nucleotides and/or other features.

(iii) Replicating the Target Nucleic Acid

The primed target nucleic acid may be replicated by an enzyme with strand-displacing activity. Examples of suitable strand-displacement polymerases include, but are not limited to, Exo-Minus Klenow DNA polymerase (i.e., large fragment of DNA Pol I that lacks both 5′→3′ and 3′→5′ exonuclease activities), Exo-Minus T7 DNA polymerase (i.e., SEQUENASE™ Version 2.0, USB Corp., Cleveland, OH), Phi29 DNA polymerase, Bst DNA polymerase, Bca polymerase, Vent DNA polymerase, 9° Nm DNA polymerase, MMLV reverse transcriptase, AMV reverse transcriptase, HIV reverse transcriptase, variants thereof, or combinations thereof. In one embodiment, the strand-displacing polymerase may be Exo-Minus Klenow DNA polymerase. In another embodiment, the strand-displacing polymerase may be MMLV reverse transcriptase. In yet another embodiment, the strand-displacing polymerase may comprise both MMLV reverse transcriptase and Exo-Minus Klenow DNA polymerase.

Alternatively, the primed target nucleic acid may be replicated via a two-step process. That is, the first strand of cDNA may be synthesized by a reverse transcriptase and then the second strand of cDNA may be synthesized by an enzyme without strand-displacing activity, such as Taq DNA polymerase.

The strand-displacing or replicating enzyme is incubated with the target nucleic acid and the plurality of degenerate oligonucleotide primers under conditions that permit hybridization between complementary sequences, as well as extension of the hybridized primer, i.e., replication of the nucleic acid. The incubation conditions are generally selected to allow hybridization between complementary sequences, but preclude hybridization between mismatched sequences (i.e., those with no or limited complementarity). The incubation conditions are also selected to optimize primer extension and promote strand-displacing activity. During replication, displaced single strands are generated that become new templates for oligonucleotide primer hybridization and primer extension. Thus, the incubation conditions generally comprise a solution of optimal pH, ionic strength, and Mg²⁺ ion concentration, with incubation at a temperature that permits both hybridization and replication.

The library synthesis buffer generally comprises a pH modifying or buffering agent that is operative at a pH of about 6.5 to about 9.5, and preferably at a pH of about 7.5. Representative examples of suitable pH modifying agents include Tris buffers, MOPS, HEPES, Bicine, Tricine, TES, or PIPES. The library synthesis buffer may comprise a monovalent salt such as NaCl, at a concentration that ranges from about 1 mM to about 200 mM. The concentration of MgCl₂ in the library synthesis buffer may range from about 5 mM to about 10 mM. The requisite mixture of deoxynucleotide triphosphates (i.e., dNTPs) may be provided in the library synthesis buffer, or it may be provided separately. The incubation temperature may range from about 12° C. to about 70° C., depending upon the polymerase used. The duration of the incubation may range from about 5 minutes to about 4 hours. In one embodiment, the incubation may comprise a single isothermal step, e.g., at about 30° C. for about 1 hour. In another embodiment, the incubation may be performed by cycling through several temperature steps (e.g., 16° C., 24° C., and 37° C.) for a short period of time (e.g., about 1-2 minutes) for a certain number of cycles (e.g., about 15-20 cycles). In yet another embodiment, the incubation may comprise sequential isothermal steps lasting from about 10 to 30 minutes. As an example, the incubation may comprise steps of 18° C. for 10 minutes, 25° C. for 10 minutes, 37° C. for 30 minutes, and 42° C. for 10 minutes. The reaction buffer may further comprise a factor that promotes stand-displacement, such as a single-stranded DNA binding protein (SSB) or a helicase. The SSB or helicase may be of bacterial, viral, or eukaryotic origin. The replication reaction may be terminated by adding a sufficient amount of EDTA to chelate the Mg²⁺ ions and/or by heat-inactivating the enzyme.

Replication of the randomly-primed target nucleic acid by a strand-displacing enzyme creates a library of overlapping molecules that range from about 100 base pairs to about 2000 base pairs in length, with an average length of about 400 to about 500 base pairs. In some embodiments, the library of replicated strands may be flanked by a constant non-degenerate end sequence that corresponds to the constant non-degenerate sequence of the plurality of oligonucleotide primers.

### (b) Amplifying the Library

The method may further comprise the step of amplifying the library through a polymerase chain reaction (PCR) process. In some embodiments, the library of replicated strands may be flanked by a constant non-degenerate end sequence, as described above. In other embodiments, at least one adaptor may be ligated to each end of the replicated strands of the library, such that the library of molecules is amplifiable. The adaptor may comprise a universal priming sequence, as described above, or a homopolymeric sequence, such as poly-G or poly-C. Suitable ligase enzymes and ligation techniques are well known in the art.

In some embodiments, PCR may be performed using a single amplification primer that is complementary to the constant end sequence of the library molecules. In other embodiments, PCR may be performed using a pair of amplification primers. In all embodiments, a thermostable DNA polymerase catalyzes the PCR amplification process. Non-limiting examples of suitable thermostable DNA polymerases include Taq DNA polymerase, Pfu DNA polymerase, Tli (also known as Vent) DNA polymerase, Tfl DNA polymerase, Tth DNA polymerase, variants thereof, and combinations thereof. The PCR process may comprise 3 steps (i.e., denaturation, annealing, and extension) or 2 steps (i.e., denaturation and annealing/extension). The temperature of the annealing or annealing/extension step can and will vary, depending upon the amplification primer. That is, its nucleotide sequence, melting temperature, and/or concentration. The temperature of the annealing or annealing/extending step may range from about 50° C. to about 75° C. In a preferred embodiment, the temperature of the annealing or annealing/extending step may be about 70° C. The duration of the PCR steps may also vary. The duration of the denaturation step may range from about 10 seconds to about 2 minutes, and the duration of the annealing or annealing/extending step may be range from about 15 seconds to about 10 minutes. The total number of cycles may also vary, depending upon the quantity and quality of the target nucleic acid. The number of cycles may range from about 5 cycles to about 50 cycles, from about 10 cycles to about 30 cycles, and more preferably from about 14 cycles to about 20 cycles.

PCR amplification of the library will generally be performed in the presence of a suitable amplification buffer. The library amplification buffer may comprise a pH modifying agent, a divalent cation, a monovalent cation, and a stabilizing agent, such as a detergent or BSA. Suitable pH modifying agents include those known in the art that will maintain the pH of the reaction from about 8.0 to about 9.5. Suitable divalent cations include magnesium and/or manganese, and suitable monovalent cations include potassium, sodium, and/or lithium. Detergents that may be included include poly(ethylene glycol)4-nonphenyl 3-sulfopropyl ether potassium salt, 3-[(3-cholamidopropyl)dimethylammonio]-1-propanesulfonate, 3-[(3-cholamidopropyl)dimethylammonio]-2-hydroxy-1-propanesulfonate, Tween 20, and Nonidet NP40. Other agents that may be included in the amplification buffer include glycerol and/or polyethylene glycol. The amplification buffer may also comprise the requisite mixture of dNTPs. In some embodiments, the PCR amplification may be performed in the presence of modified nucleotide such that the amplified library is labeled for downstream analyses. Non-limiting examples of suitable modified nucleotides include fluorescently labeled nucleotides, aminoallyl-dUTP, bromo-dUTP, or digoxigenin-labeled nucleotide triphosphates.

The percentage of target nucleic acid that is represented in the amplified library can and will vary, depending upon the type and quality of the target nucleic acid. The amplified library may represent at least about 50%, about 60%, about 70%, about 80%, about 85%, about 90%, about 95%, about 97%, about 99%, or about 99.5% of the target nucleic acid. The fold of amplification may also vary, depending upon the target nucleic acid. The fold of amplification may be about 100-fold, 300-fold, about 1000-fold, about 10,000-fold, about 100,000-fold, or about 1,000,000-fold. For example, about 5 ng to about 10 ng of a target nucleic acid may be amplified into about 5 μg to about 50 μg of amplified library molecules. Furthermore, the amplified library may be re-amplified by PCR.

The amplified library may be purified to remove residual amplification primers and nucleotides prior to subsequent uses. Methods of nucleic acid purification, such as spin column chromatography or filtration techniques, are well known in the art.

The downstream use of the amplified library may vary. Non-limiting uses of the amplified library include quantitative real-time PCR, microarray analysis, sequencing, restriction fragment length polymorphism (RFLP) analysis, single nucleotide polymorphism (SNP) analysis, microsatellite analysis, short tandem repeat (STR) analysis, comparative genomic hybridization (CGH), fluorescent in situ hybridization (FISH), and chromatin immunoprecipitation (ChiP).

### (III) Kit for Amplifying a Population of Target Nucleic Acids

A further aspect of the invention encompasses a kit for amplifying a population of target nucleic acids. The kit comprises a plurality of oligonucleotide primers, as defined above in section (I), and a replicating enzyme, as defined above in section (II)(a)(iii).

In a preferred embodiment, the plurality of oligonucleotide primers of the kit may comprise the formula NₘXₚ, NₘZ_{q}, or a combination thereof, wherein N, X, and Z are degenerate nucleotides as defined above, m is from 2 to 13, p and q are each from 1 to 11, and the sum total of the two integers is from 6 to 14, and the at least two N residues are separated by at least one X or Z residue. In an exemplary embodiment, the plurality of oligonucleotide primers of the kit comprise the formula NₘXₚ, NₘZ_{q}, or a combination thereof, wherein N, X, and Z are degenerate nucleotides as defined above, m is from 2 to 8, p and q are each from 1 to 7, the sum total of m and p or m and q is 9, the at least two N residues are separated by at least one X or Z residue, and there are no more than three consecutive N residues. In preferred embodiments, X is D and Y is K. In an especially preferred embodiment, the plurality of oligonucleotide primers of the kit have the following (5′-3′) sequences: KNNNKNKNK, NKNNKNNKK, and NNNKNKKNK. In some embodiments, the plurality of oligonucleotide primers may further comprise an oligo dT primer. The plurality of oligonucleotide primers of the kit may also further comprise a constant non-degenerate sequence at the 5′ end of each primer, as described above in section (I)(b).

The kit may further comprise a library synthesis buffer, as defined in section (II)(a)(iii). Another optional component of the kit is means to fragment a target nucleic acid, as described above in section (II)(a)(i). The kit may also further comprise a thermostable DNA polymerase, at least one amplification primer, and a library amplification buffer, as described in section (II)(b).

### Definitions

To facilitate understanding of the invention, a number of terms are defined below.

The terms “complementary or complementarity,” as used herein, refer to the ability to form at least one Watson-Crick base pair through specific hydrogen bonds. The terms “non-complementary or non-complementarity” refer to the inability to form at least one Watson-Crick base pair through specific hydrogen bonds.

“Genomic DNA” refers to one or more chromosomal polymeric deoxyribonucleic acid molecules occurring naturally in the nucleus or an organelle (e.g., mitochondrion, chloroplast, or kinetoplast) of a eukaryotic cell, a eubacterial cell, an archaeal cell, or a virus. These molecules contain sequences that are transcribed into RNA, as well as sequences that are not transcribed into RNA.

The term “hybridization,” as used herein, refers to the process of hydrogen bonding, or base pairing, between the bases comprising two complementary single-stranded nucleic acid molecules to form a double-stranded hybrid. The “stringency” of hybridization is typically determined by the conditions of temperature and ionic strength. Nucleic acid hybrid stability is generally expressed as the melting temperature or Tₘ, which is the temperature at which the hybrid is 50% denatured under defined conditions. Equations have been derived to estimate the Tₘ of a given hybrid; the equations take into account the G+C content of the nucleic acid, the nature of the hybrid (e.g., DNA:DNA, DNA:RNA, etc.), the length of the nucleic acid probe, etc. (e.g., Sambrook et al. (1989) Molecular Cloning: A Laboratory Manual, Cold Spring Harbor Press, Cold Spring Harbor, NY, chapter 9). In many reactions that are based upon hybridization, e.g., polymerase reactions, amplification reactions, ligation reactions, etc., the temperature of the reaction typically determines the stringency of the hybridization.

The term “primer,” as generally used, refers to a nucleic acid strand or an oligonucleotide having a free 3′ hydroxyl group that serves as a starting point for DNA replication.

The term “transcriptome,” as used herein, is defined as the set of all RNA molecules expressed in one cell or a population of cells. The set of RNA molecules may include messenger RNAs and/or microRNAs and other small RNAs. The term may refer to the total set of RNA molecules in a given organism, or to the specific subset of RNA molecules present in a particular cell type.

### EXAMPLES

The following examples are included to demonstrate various embodiments of the invention. It should be appreciated by those of skill in the art that the techniques disclosed in the examples that follow represent techniques discovered by the inventors to function well in the practice of the invention. However, those of skill in the art should, in light of the present disclosure, appreciate that many changes may be made in the specific embodiments that are disclosed and still obtain a like or similar result without departing from the spirit and scope of the invention, therefore all matter set forth in the above description and in the examples given below, shall be interpreted as illustrative and not in a limiting sense.

### Example 1. Analysis of a D9 Library Synthesis Primer

In an attempt to increase the degeneracy of primers used in WGA and WTA applications, a library synthesis primer was synthesized whose semi-random region comprised nine D residues (D9). The primer also comprised a constant (universal) 5′ region. The ability of this primer to efficiently amplify a large number of amplicons was compared to that of a standard library synthesis primer whose semi-random region comprised nine K residues (K9) (e.g., that provided in the Rubicon TRANSPLEX™ Whole Transcriptome Amplification (WTA) Kit, Sigma-Aldrich, St. Louis, MO). Both K9 and D9 amplified cDNAs were compared to unamplified cDNA by qPCR and microarray analyses.
(a) Unamplified Control cDNA Synthesis

Single-stranded cDNA was prepared from 30 micrograms of total human liver RNA (cat.# 7960; Ambion, Austin, TX) and Universal Human Reference (UHR) total RNA (cat.# 74000; Stratagene, La Jolla, CA) at a concentration of 1 microgram of total RNA per 50-microliter reaction, using 1 μM oligo dT₁₉ (SEQ ID NO: 53) primer following the procedure described for MMLV-reverse transcriptase (cat.# M1302; Sigma-Aldrich).
(b) D-amplified cDNA Synthesis

One microgram of human liver or UHR total RNA per 25-microliters and 1 μM of an oligo dT primer (5′-GTAGGTTGAGGATAGGAGGGTTAGGT19-3′; SEQ ID NO:1) were incubated at 70° C. for 5 minutes, quick cooled on ice, and followed immediately by addition of 10 unit/microliter MMLV-reverse transcriptase (Sigma-Aldrich), 1× PCR Buffer (cat.# P2192; Sigma-Aldrich), magnesium chloride(cat.# M8787; Sigma-Aldrich) added to 3 mM final concentration, 500 μM dNTPs, and 2.5% (volume) Ribonuclease Inhibitor (cat.#R2520; Sigma-Aldrich) and incubated at 37° C. for 5 minutes, 42° C. for 45 minutes, 94° C. for 5 minutes, and quick-chilled on ice.

Complementary second cDNA strand was synthesized using 1 μM of the D9 library synthesis primer (5′-GTAGGTTGAGGATAGGAGGGTTAGGD9-3′; SEQ ID NO:2), 0.165 units/microliter JUMPSTART™ Taq DNA polymerase (cat.# D3443; Sigma-Aldrich), 0.18 unit/microliter Klenow exo-minus DNA polymerase (cat.# 7057Z; USB, Cleveland, OH), 1× PCR Buffer (see above), 5.5 mM added magnesium chloride (see above) and 500 μM dNTPs. The mixture was incubated at 18° C. for 5 minutes, 25° C. for 5 minutes, 37° C. for 5 minutes, and 72° C. for 15 minutes.

Double-stranded cDNAs were amplified using 0.05 units/microliter JUMPSTART™ Taq (see above), 1× PCR Buffer (cat.# D4545, without magnesium chloride, Sigma-Aldrich), 1.5 mM magnesium chloride (see above), 200 μM dNTPs and 2 μM of the universal primer 5′-GTAGGTTGAGGATAGGAGGGTTAGG-3′ (SEQ ID NO:3). Thermocycling parameters were: 94° C. for 90 seconds, then seventeen cycles of 94° C. for 30 seconds, 65° C. for 30 seconds, and 72° C. for 2 minutes.
(c) K-amplified cDNA Synthesis

Amplified cDNA was prepared from 0.2 micrograms total RNAs (see above) using the synthesis components and procedures of the Rubicon Transplex™M WTA Kit (see above).
(d) RNA Removal and cDNA Purification

Total RNA template in unamplified control cDNA and amplified cDNAs was degraded by addition (in sequence) of ⅓ final cDNA/amplification reaction volume of 0.5 M EDTA and ⅓ final cDNA/amplification reaction volume of 1 M NaOH, with incubation at 65° C. for 15 minutes. Reactions were then neutralized with ⅚ final cDNA/amplification reaction volume of 1 M Tris HCl, pH 7.4, and purified using the GenElute PCR Cleanup kit as described (cat.# NA1020; Sigma-Aldrich).
(e) Quantitative PCR (@PCR) Analysis

Amplified cDNAs and unamplified control cDNAs were analyzed by real-time quantitative PCR, using conditions prescribed for 2× SYBR® Green JUMPSTART™ Taq (cat.# S4438; Sigma-Aldrich), with 250 nM human primers pairs (see Table 1). Cycling conditions were 1 cycle at 94° C. for 1.5 minutes, and 30 cycles at 94° C. for 30 seconds; 60° C. for 30 seconds; and 72° C. for 2.5 minutes.

TABLE 1
Primers used in qPCR.

| | | Primer 1 | SEQ | Primer 2 | SEQ |
|---|---|---|---|---|---|
| Primer | | Sequence | ID | Sequence | ID |
| Set | Gene | (5′-3′) | NO: | (5′-3′) | NO: |
| 1 | M55047 | TGCTTAGA | 4 | CTTGACAA | 5 |
| | | CCCGTAGT | | AATGCTGT | |
| | | TTCC | | GTTCC | |
| 2 | sts-N90764 | CGTTTAAT | 6 | AGCCAAGT | 7 |
| | | TCTGTGGC | | ACCCCGAC | |
| | | CAGG | | TACG | |
| 3 | WI-13668 | TGTTAACA | 8 | TGATTAAT | 9 |
| | | ATTTGCAT | | TTGCGAGA | |
| | | AACAAAAG | | CTAACTTT | |
| | | C | | G | |
| 4 | shgc-79529 | GTTTCGAA | 10 | CACAATCA | 11 |
| | | TCCCAGGA | | GCAACAAA | |
| | | ATTAAGC | | ATCATCC | |
| 5 | shgc-11640 | GCAAACAA | 12 | TTCTCCCA | 13 |
| | | AGCATGCT | | GCTTTGAG | |
| | | TCAA | | ACGT | |
| 6 | SHGC-36464 | TATTTAAA | 14 | TGGTGTAA | 15 |
| | | ATGTGGGC | | ATAAAGAC | |
| | | AAGATATC | | CTTGCTAT | |
| | | A | | C | |
| 7 | kiaa0108 | TTTGTTAC | 16 | CAACCATC | 17 |
| | | TTGCTACC | | ATCTTCCA | |
| | | CTGAG | | CAGTC | |
| 8 | stSG53466 | AGACCACA | 18 | GAATTTTG | 19 |
| | | CCAGAAAC | | GTTTCTTG | |
| | | CCTG | | CTTTGG | |
| 9 | SHGC153324 | CCAGGGTT | 20 | GATTTCTA | 21 |
| | | CGAATCTC | | AACTTACG | |
| | | AGTCTTA | | GCCCCAC | |
| 10 | 1314 | AAAGAGTG | 22 | TTATCTGA | 23 |
| | | TCTTGTCT | | GCCCTTAA | |
| | | TGACTTAT | | TAGTAAAT | |
| | | C | | C | |
| 11 | stSG62388 | AATCAAAA | 24 | TTCAGTGT | 25 |
| | | GGCCAACA | | TAATGGAG | |
| | | GTGG | | CCAGG | |
| 12 | sts- | TCTCAGAG | 26 | CCTGCACT | 27 |
| | AA035504 | CAGAGTTT | | TGGACCTG | |
| | | GGGC | | ACC | |

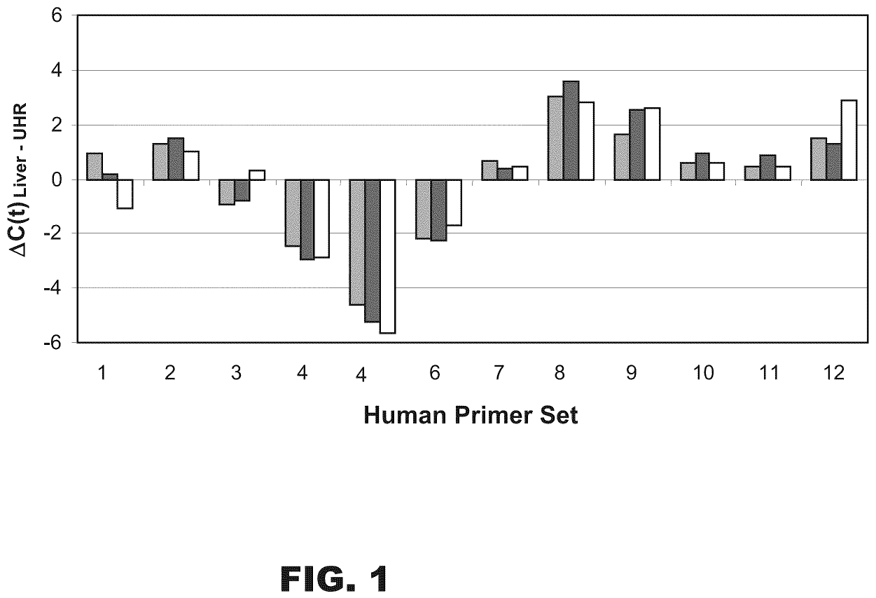

The C(t) value, which represents the PCR cycle during which the fluorescence exceeded a defined threshold level, was determined for each reaction. The average delta C(t) [ΔC(t)] was calculated and subtracted from individual ΔC(t) values for that PCR template type. FIG. 1 presents the ΔC(t)_{Liver-UHR} for each population of cDNAs as a function of the different primer sets. The results indicate that the ratio of human liver and UHR cDNA amplicon concentrations, as represented by the ΔC(t)s, for the D-amplified cDNAs and the K-amplified cDNAs closely reflected the ratio of initial mRNA levels represented in the unamplified total RNA.
(f) Microarray Analysis

Target cDNA was labeled using the Kreatech ULS™ system (Kreatech Biotechnology, Amsterdam, Netherlands; the labeling was performed by Mogene, LC, NIDUS Center for Scientific Enterprise, 893 North Warson Road, Saint Louis, MO, 63141). Purified unamplified cDNA, D-amplified cDNA and K-amplified cDNA were submitted to Mogene, LC for microarray analysis. For this, 750 nanograms of target were incubated with the Agilent Whole Genome Chip (cat.# G4112A; Agilent Technologies, Santa Clara, CA).

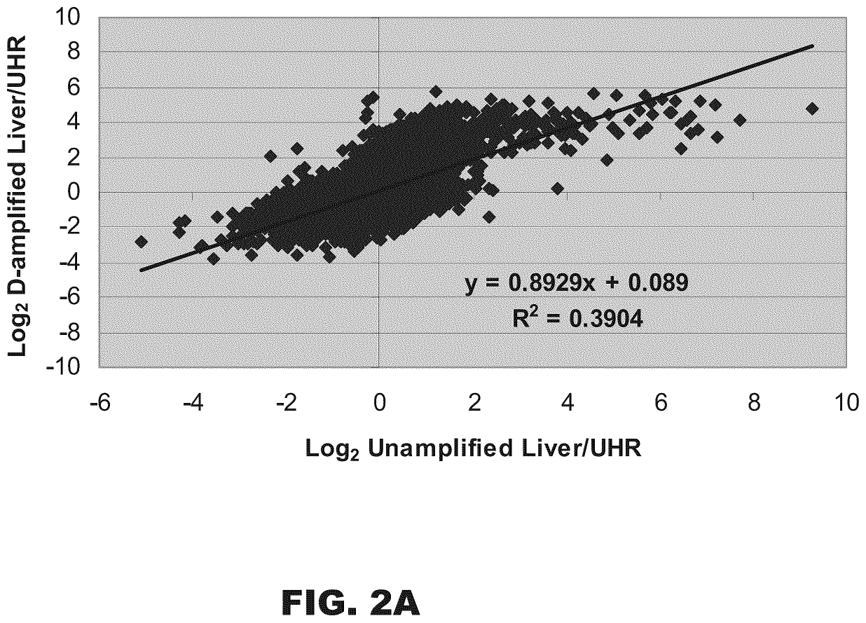

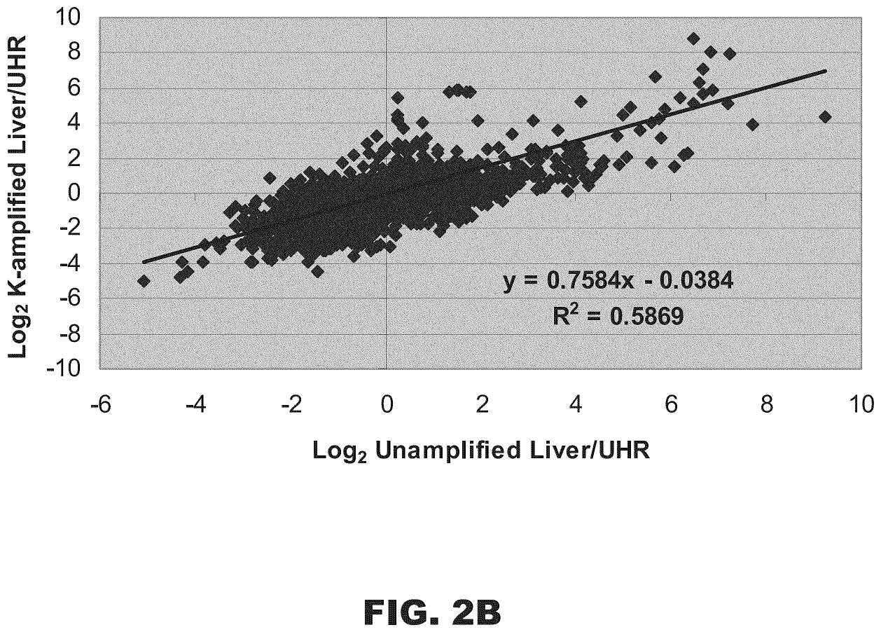

FIG. 2 presents the ratio spot intensities representing human liver and UHR target for each array probe. The log base 2 ratios of amplified cDNAs targets were plotted against the log base 2 ratio for unamplified cDNA target. Only intensities of approximately 5X background (>250) were included in this analysis. The results reveal that D-amplified (FIG. 2A) and K-amplified *FIG. 2B) cDNAs had similar profiles.

### Example 2. Selection of 384 Highly Degenerate Primers

To further increase the degeneracy of library synthesis primers, the semi-random region was modified to include N residues, as well as either D or K residues. It was reasoned that addition of Ns would increased the sequence diversity, and interruption of the Ns with K or D residues would reduce intramolecular and intermolecular interactions among the primers. Table 2 lists 256 possible K interrupted N sequences (including the control K9 sequence, also called 1K9) and Table 3 lists 256 possible D interrupted N sequences (including the control D9 sequence, also called 1D9).

In an effort to minimize the number of primers to investigate, and provide a workable example, it was decided to limit the number of primers to evaluate to 384. The first cut was to eliminate any sequence containing 4 or more contiguous N residues, as it was assumed that four or more degenerate Ns could provide a substantial opportunity for primer dimer formation. This reduced the number of K or D interrupted N sequences from 256 to 208. The remaining 16 primers (i.e., 208 to 192) were eliminated on the basis of 3′ diversity and self-complementarity. Of the sixteen, six comprised the eight possible N₁X₈ sequences where maximal 3′ degeneracy was maintained by keeping the two candidate sequences with N near the 3′ end saving the penultimate position because 50% of the pool would be self complimentary at the final two 3′ nucleotides. The remaining 10 sequences were eliminated on the basis of self-complementarity (i.e., degenerate sequences that were palindromic about a central N pairing K/D's with N, e.g. NKNNNKKNK, NNKKNNNKK, etc.). Table 4 lists the final 384 interrupted N sequences that were selected for subsequent screening.

TABLE 2
Possible 9-mer KN sequences.

| | | | | |
|---|---|---|---|---|
| KKKKKKKKK | KKNKNNKKK | NNNKKNNKK | KNKNNKKNK | NKNNKKNNK |
| NKKKKKKKK | NKNKNNKKK | KKKNKNNKK | NNKNNKKNK | KNNNKKNNK |
| KNKKKKKKK | KNNKNNKKK | NKKNKNNKK | KKNNNKKNK | NNNNKKNNK |
| NNKKKKKKK | NNNKNNKKK | KNKNKNNKK | NKNNNKKNK | KKKKNKNNK |
| KKNKKKKKK | KKKNNNKKK | NNKNKNNKK | KNNNNKKNK | NKKKNKNNK |
| NKNKKKKKK | NKKNNNKKK | KKNNKNNKK | NNNNNKKNK | KNKKNKNNK |
| KNNKKKKKK | KNKNNNKKK | NKNNKNNKK | KKKKKNKNK | NNKKNKNNK |
| NNNKKKKKK | NNKNNNKKK | KNNNKNNKK | NKKKKNKNK | KKNKNKNNK |
| KKKNKKKKK | KKNNNNKKK | NNNNKNNKK | KNKKKNKNK | NKNKNKNNK |
| NKKNKKKKK | NKNNNNKKK | KKKKNNNKK | NNKKKNKNK | KNNKNKNNK |
| KNKNKKKKK | KNNNNNKKK | NKKKNNNKK | KKNKKNKNK | NNNKNKNNK |
| NNKNKKKKK | NNNNNNKKK | KNKKNNNKK | NKNKKNKNK | KKKNNKNNK |
| KKNNKKKKK | KKKKKKNKK | NNKKNNNKK | KNNKKNKNK | NKKNNKNNK |
| NKNNKKKKK | NKKKKKNKK | KKNKNNNKK | NNNKKNKNK | KNKNNKNNK |
| KNNNKKKKK | KNKKKKNKK | NKNKNNNKK | KKKNKNKNK | NNKNNKNNK |
| NNNNKKKKK | NNKKKKNKK | KNNKNNNKK | NKKNKNKNK | KKNNNKNNK |
| KKKKNKKKK | KKNKKKNKK | NNNKNNNKK | KNKNKNKNK | NKNNNKNNK |
| NKKKNKKKK | NKNKKKNKK | KKKNNNNKK | NNKNKNKNK | KNNNNKNNK |
| KNKKNKKKK | KNNKKKNKK | NKKNNNNKK | KKNNKNKNK | NNNNNKNNK |
| NNKKNKKKK | NNNKKKNKK | KNKNNNNKK | NKNNKNKNK | KKKKKNNNK |
| KKNKNKKKK | KKKNKKNKK | NNKNNNNKK | KN NNKNKNK | NKKKKNNNK |
| NKNKNKKKK | NKKNKKNKK | KKNNNNNKK | NNNNKNKNK | KNKKKNNNK |
| KNNKNKKKK | KNKNKKNKK | NKNNNNNKK | KKKKNNKNK | NNKKKNNNK |
| NNNKNKKKK | NNKNKKNKK | KNNNNNNKK | NKKKNNKNK | KKNKKNNNK |
| KKKNNKKKK | KKNNKKNKK | NNNNNNNKK | KNKKNNKNK | NKNKKNNNK |
| NKKNNKKKK | NKNNKKNKK | KKKKKKKNK | NNKKNNKNK | KNNKKNNNK |
| KNKNNKKKK | KNNNKKNKK | NKKKKKKNK | KKNKNNKNK | NNNKKNNNK |
| NNKNNKKKK | NNNNKKNKK | KNKKKKKNK | NKNKNNKNK | KKKNKNNNK |
| KKNNNKKKK | KKKKNKNKK | NNKKKKKNK | KNNKNNKNK | NKKNKNNNK |
| NKNNNKKKK | NKKKNKNKK | KKNKKKKNK | NNNKNNKNK | KNKNKNNNK |
| KNNNNKKKK | KNKKNKNKK | NKNKKKKNK | KKKNNNKNK | NNKNKNNNK |
| NNNNNKKKK | NNKKNKNKK | KNNKKKKNK | NKKNNNKNK | KKNNKNNNK |
| KKKKKNKKK | KKNKNKNKK | NNNKKKKNK | KNKNNNKNK | NKNNKNNNK |
| NKKKKNKKK | NKNKNKNKK | KKKNKKKNK | NNKNNNKNK | KNNNKNNNK |
| KNKKKNKKK | KNNKNKNKK | NKKNKKKNK | KKNNNNKNK | NNNNKNNNK |
| NNKKKNKKK | NNNKNKNKK | KNKNKKKNK | NKNNNNKNK | KKKKNNNNK |
| KKNKKNKKK | KKKNNKNKK | NNKNKKKNK | KNNNNNKNK | NKKKNNNNK |
| NKNKKNKKK | NKKNNKNKK | KKNNKKKNK | NNNNNNKNK | KNKKNNNNK |
| KNNKKNKKK | KNKNNKNKK | NKNNKKKNK | KKKKKKNNK | NNKKNNNNK |
| NNNKKNKKK | NNKNNKNKK | KNNNKKKNK | NKKKKKNNK | KKNKNNNNK |
| KKKNKNKKK | KKNNNKNKK | NNNNKKKNK | KNKKKKNNK | NKNKNNNNK |
| NKKNKNKKK | NKNNNKNKK | KKKKNKKNK | NNKKKKNNK | KNNKNNNNK |
| KNKNKNKKK | KNNNNKNKK | NKKKNKKNK | KKNKKKNNK | NNNKNNNNK |
| NNKNKNKKK | NNNNNKNKK | KNKKNKKNK | NKNKKKNNK | KKKNNNNNK |
| KKNNKNKKK | KKKKKNNKK | NNKKNKKNK | KNNKKKNNK | NKKNNNNNK |
| NKNNKNKKK | NKKKKNNKK | KKNKNKKNK | NNNKKKNNK | KNKNNNNNK |
| KNNNKNKKK | KNKKKNNKK | NKNKNKKNK | KKKNKKNNK | NNKNNNNNK |
| NNNNKNKKK | NNKKKNNKK | KNNKNKKNK | NKKNKKNNK | KKNNNNNNK |
| KKKKNNKKK | KKNKKNNKK | NNNKNKKNK | KNKNKKNNK | NKNNNNNNK |
| NKKKNNKKK | NKNKKNNKK | KKKNNKKNK | NNKNKKNNK | KNNNNNNNK |
| KNKKNNKKK | KNNKKNNKK | NKKNNKKNK | KKNNKKNNK | NNNNNNNNK |
| NNKKNNKKK | | | | |

TABLE 3
Possible 9-mer DN sequences.

| | | | | |
|---|---|---|---|---|
| DDDDDDDDD | DDNDNNDDD | NNNDDNNDD | DNDNNDDND | NDNNDDNND |
| NDDDDDDDD | NDNDNNDDD | DDDNDNNDD | NNDNNDDND | DNNNDDNND |
| DNDDDDDDD | DNNDNNDDD | NDDNDNNDD | DDNNNDDND | NNNNDDNND |
| NNDDDDDDD | NNNDNNDDD | DNDNDNNDD | NDNNNDDND | DDDDNDNND |
| DDNDDDDDD | DDDNNNDDD | NNDNDNNDD | DNNNNDDND | NDDDNDNND |
| NDNDDDDDD | NDDNNNDDD | DDNNDNNDD | NNNNNDDND | DNDDNDNND |
| DNNDDDDDD | DNDNNNDDD | NDNNDNNDD | DDDDDNDND | NNDDNDNND |
| NNNDDDDDD | NNDNNNDDD | DNNNDNNDD | NDDDDNDND | DDNDNDNND |
| DDDNDDDDD | DDNNNNDDD | NNNNDNNDD | DNDDDNDND | NDNDNDNND |
| NDDNDDDDD | NDNNNNDDD | DDDDNNNDD | NNDDDNDND | DNNDNDNND |
| DNDNDDDDD | DNNNNNDDD | NDDDNNNDD | DDNDDNDND | NNNDNDNND |
| NNDNDDDDD | NNNNNNDDD | DNDDNNNDD | NDNDDNDND | DDDNNDNND |
| DDNNDDDDD | DDDDDDNDD | NNDDNNNDD | DNNDDNDND | NDDNNDNND |
| NDNNDDDDD | NDDDDDNDD | DDNDNNNDD | NNNDDNDND | DNDNNDNND |
| DNNNDDDDD | DNDDDDNDD | NDNDNNNDD | DDDNDNDND | NNDNNDNND |
| NNNNDDDDD | NNDDDDNDD | DNNDNNNDD | NDDNDNDND | DDNNNDNND |
| DDDDNDDDD | DDNDDDNDD | NNNDNNNDD | DNDNDNDND | NDNNNDNND |
| NDDDNDDDD | NDNDDDNDD | DDDNNNNDD | NNDNDNDND | DNNNNDNND |
| DNDDNDDDD | DNNDDDNDD | NDDNNNNDD | DDNNDNDND | NNNNNDNND |
| NNDDNDDDD | NNNDDDNDD | DNDNNNNDD | NDNNDNDND | DDDDDNNND |
| DDNDNDDDD | DDDNDDNDD | NNDNNNNDD | DNNNDNDND | NDDDDNNND |
| NDNDNDDDD | NDDNDDNDD | DDNNNNNDD | NNNNDNDND | DNDDDNNND |
| DNNDNDDDD | DNDNDDNDD | NDNNNNNDD | DDDDNNDND | NNDDDNNND |
| NNNDNDDDD | NNDNDDNDD | DNNNNNNDD | NDDDNNDND | DDNDDNNND |
| DDDNNDDDD | DDNNDDNDD | NNNNNNNDD | DNDDNNDND | NDNDDNNND |
| NDDNNDDDD | NDNNDDNDD | DDDDDDDND | NNDDNNDND | DNNDDNNND |
| DNDNNDDDD | DNNNDDNDD | NDDDDDDND | DDNDNNDND | NNNDDNNND |
| NNDNNDDDD | NNNNDDNDD | DNDDDDDND | NDNDNNDND | DDDNDNNND |
| DDNNNDDDD | DDDDNDNDD | NNDDDDDND | DNNDNNDND | NDDNDNNND |
| NDNNNDDDD | NDDDNDNDD | DDNDDDDND | NNNDNNDND | DNDNDNNND |
| DNNNNDDDD | DNDDNDNDD | NDNDDDDND | DDDNNNDND | NNDNDNNND |
| NNNNNDDDD | NNDDNDNDD | DNNDDDDND | NDDNNNDND | DDNNDNNND |
| DDDDDNDDD | DDNDNDNDD | NNNDDDDND | DNDNNNDND | NDNNDNNND |
| NDDDDNDDD | NDNDNDNDD | DDDNDDDND | NNDNNNDND | DNNNDNNND |
| DNDDDNDDD | DNNDNDNDD | NDDNDDDND | DDNNNNDND | NNNNDNNND |
| NNDDDNDDD | NNNDNDNDD | DNDNDDDND | NDNNNNDND | DDDDNNNND |
| DDNDDNDDD | DDDNNDNDD | NNDNDDDND | DNNNNNDND | NDDDNNNND |
| NDNDDNDDD | NDDNNDNDD | DDNNDDDND | NNNNNNDND | DNDDNNNND |
| DNNDDNDDD | DNDNNDNDD | NDNNDDDND | DDDDDDNND | NNDDNNNND |
| NNNDDNDDD | NNDNNDNDD | DNNNDDDND | NDDDDDNND | DDNDNNNND |
| DDDNDNDDD | DDNNNDNDD | NNNNDDDND | DNDDDDNND | NDNDNNNND |
| NDDNDNDDD | NDNNNDNDD | DDDDNDDND | NNDDDDNND | DNNDNNNND |
| DNDNDNDDD | DNNNNDNDD | NDDDNDDND | DDNDDDNND | NNNDNNNND |
| NNDNDNDDD | NNNNNDNDD | DNDDNDDND | NDNDDDNND | DDDNNNNND |
| DDNNDNDDD | DDDDDNNDD | NNDDNDDND | DNNDDDNND | NDDNNNNND |
| NDNNDNDDD | NDDDDNNDD | DDNDNDDND | NNNDDDNND | DNDNNNNND |
| DNNNDNDDD | DNDDDNNDD | NDNDNDDND | DDDNDDNND | NNDNNNNND |
| NNNNDNDDD | NNDDDNNDD | DNNDNDDND | NDDNDDNND | DDNNNNNND |
| DDDDNNDDD | DDNDDNNDD | NNNDNDDND | DNDNDDNND | NDNNNNNND |
| NDDDNNDDD | NDNDDNNDD | DDDNNDDND | NNDNDDNND | DNNNNNNND |
| DNDDNNDDD | DNNDDNNDD | NDDNNDDND | DDNNDDNND | NNNNNNNND |
| NNDDNNDDD | | | | |

TABLE 4
The 384 Interrupted N Sequences Selected for Further Screening.

| | | | | | Sequence |
|---|---|---|---|---|---|
| Name | Sequence (5′-3′) | Name | Sequence (5′-3′) | Name | (5′-3′) |
| 1K3 | KNNNKNNNK | 24K6 | KNKNNKKKK | 25D5 | DNDNDNDND |
| 2K3 | NKNNKNNNK | 25K6 | KNNKNKKKK | 26D5 | DNNDDNDND |
| 3K3 | NNKNNNKNK | 26K6 | KNKKKNNKK | 27D5 | DNNNDNDDD |
| 4K3 | NNNKNKNNK | 27K6 | KNKKKNKNK | 28D5 | DNDNDDNND |
| 5K3 | NNKNKNNNK | 28K6 | KNKNKNKKK | 29D5 | DNNDDDNND |
| 6K3 | NNNKKNNNK | 29K6 | KNNKKNKKK | 30D5 | DNNNDDNDD |
| 1K4 | KKNNNKNNK | 30K6 | KNKKKKNNK | 31D5 | DNNNDDDND |
| 2K4 | KKNNKNNNK | 31K6 | KNKNKKNKK | 32D5 | NDDDNNNDD |
| 3K4 | KNNKNNNKK | 32K6 | KNNKKKNKK | 33D5 | NDDDNNDND |
| 4K4 | KNKNNNKNK | 33K6 | KNKNKKKNK | 34D5 | NDDNNNDDD |
| 5K4 | KNNKNNKNK | 34K6 | KNNKKKKNK | 35D5 | NDNDNNDDD |
| 6K4 | KNKNNKNNK | 35K6 | KNNNKKKKK | 36D5 | NDDDNDNND |
| 7K4 | KNNKNKNNK | 36K6 | NKKKNNKKK | 37D5 | NDDNNDNDD |
| 8K4 | KNKNKNNNK | 37K6 | NKKKNKNKK | 38D5 | NDNDNDNDD |
| 9K4 | KNNKKNNNK | 38K6 | NKKKNKKNK | 39D5 | NDDNNDDND |
| 10K4 | KNNNKNNKK | 39K6 | NKKNNKKKK | 40D5 | NDNDNDDND |
| 11K4 | KNNNKNKNK | 40K6 | NKNKNKKKK | 41D5 | NDNNNDDDD |
| 12K4 | KNNNKKNNK | 41K6 | NKKKKNNKK | 42D5 | NDDDDNNND |
| 13K4 | NKNKNNNKK | 42K6 | NKKKKNKNK | 43D5 | NDDNDNNDD |
| 14K4 | NKKNNNKNK | 43K6 | NKKNKNKKK | 44D5 | NDNDDNNDD |
| 15K4 | NKNKNKNNK | 44K6 | NKNKKNKKK | 45D5 | NDDNDNDND |
| 16K4 | NKNNNKNKK | 45K6 | NKKKKKNNK | 46D5 | NDNDDNDND |
| 17K4 | NKKNKNNNK | 46K6 | NKKNKKNKK | 47D5 | NDNNDNDDD |
| 18K4 | NKNKKNNNK | 47K6 | NKNKKKNKK | 48D5 | NDDNDDNND |
| 19K4 | NKNNKNNKK | 48K6 | NKKNKKKNK | 49D5 | NDNDDDNND |
| 20K4 | NKNNKNKNK | 49K6 | NKNKKKKNK | 50D5 | NDNNDDNDD |
| 21K4 | NKNNKKNNK | 50K6 | NKNNKKKKK | 51D5 | NDNNDDDND |
| 22K4 | NNKKNNKNK | 51K6 | NNKKNKKKK | 52D5 | NNDDNNDDD |
| 23K4 | NNKNNNKKK | 52K6 | NNKKKNKKK | 53D5 | NNDDNDNDD |
| 24K4 | NNKKNKNNK | 53K6 | NNKKKKNKK | 54D5 | NNDDNDDND |
| 25K4 | NNNKNKNKK | 54K6 | NNKKKKKNK | 55D5 | NNDNNDDDD |
| 26K4 | NNKNNKKNK | 55K6 | NNKNKKKKK | 56D5 | NNNDNDDDD |
| 27K4 | NNNKNKKNK | 56K6 | NNNKKKKKK | 57D5 | NNDDDNNDD |
| 28K4 | NNKKKNNNK | 1K7 | KKKKNNKKK | 58D5 | NNDDDNDND |
| 29K4 | NNKNKNNKK | 2K7 | KKKKNKNKK | 59D5 | NNDNDNDDD |
| 30K4 | NNNKKNNKK | 3K7 | KKKKNKKNK | 60D5 | NNNDDNDDD |
| 31K4 | NNKNKNKNK | 4K7 | KKKNNKKKK | 61D5 | NNDDDDNND |
| 32K4 | NNNKKNKNK | 5K7 | KKNKNKKKK | 62D5 | NNDNDDNDD |
| 33K4 | NNKNKKNNK | 6K7 | KKKKKNNKK | 63D5 | NNNDDDNDD |
| 34K4 | NNNKKKNNK | 7K7 | KKKKKNKNK | 64D5 | NNDNDDDND |
| 1K5 | KKNKNNNKK | 8K7 | KKKNKNKKK | 65D5 | NNNDDDDND |
| 2K5 | KKKNNNKNK | 9K7 | KKNKKNKKK | 1D6 | DDDDNNNDD |
| 3K5 | KKNKNNKNK | 10K7 | KKKKKKNNK | 2D6 | DDDDNNDND |
| 4K5 | KKKNNKNNK | 11K7 | KKKNKKNKK | 3D6 | DDDNNNDDD |
| 5K5 | KKNKNKNNK | 12K7 | KKNKKKNKK | 4D6 | DDNDNNDDD |
| 6K5 | KKNNNKNKK | 13K7 | KKKNKKKNK | 5D6 | DDDDNDNND |
| 7K5 | KKNNNKKNK | 14K7 | KKNKKKKNK | 6D6 | DDDNNDNDD |
| 8K5 | KKKNKNNNK | 15K7 | KKNNKKKKK | 7D6 | DDNDNDNDD |
| 9K5 | KKNKKNNNK | 16K7 | KNKKNKKKK | 8D6 | DDDNNDDND |
| 10K5 | KKNNKNNKK | 17K7 | KNKKKNKKK | 9D6 | DDNDNDDND |
| 11K5 | KKNNKNKNK | 18K7 | KNKKKKNKK | 10D6 | DDNNNDDDD |
| 12K5 | KKNNKKNNK | 19K7 | KNKKKKKNK | 11D6 | DDDDDNNND |
| 13K5 | KNKKNNNKK | 20K7 | KNKNKKKKK | 12D6 | DDDNDNNDD |
| 14K5 | KNKKNNKNK | 21K7 | KNNKKKKKK | 13D6 | DDNDDNNDD |
| 15K5 | KNKNNNKKK | 22K7 | NKKKNKKKK | 14D6 | DDDNDNDND |
| 16K5 | KNNKNNKKK | 23K7 | NKKKKNKKK | 15D6 | DDNDDNDND |
| 17K5 | KNKKNKNNK | 24K7 | NKKKKKNKK | 16D6 | DDNNDNDDD |
| 18K5 | KNKNNKNKK | 25K7 | NKKKKKKNK | 17D6 | DDDNDDNND |
| 19K5 | KNNKNKNKK | 26K7 | NKKNKKKKK | 18D6 | DDNDDDNND |
| 20K5 | KNKNNKKNK | 27K7 | NKNKKKKKK | 19D6 | DDNNDDNDD |
| 21K5 | KNNKNKKNK | 28K7 | NNKKKKKKK | 20D6 | DDNNDDDND |
| 22K5 | KNKKKNNNK | 1K8 | KKKKKNKKK | 21D6 | DNDDNNDDD |
| 23K5 | KNKNKNNKK | 2K8 | KKKKKKNKK | 22D6 | DNDDNDNDD |
| 24K5 | KNNKKNNKK | 1K9 | KKKKKKKKK | 23D6 | DNDDNDDND |
| 25K5 | KNKNKNKNK | 1D3 | DNNNDNNND | 24D6 | DNDNNDDDD |
| 26K5 | KNNKKNKNK | 2D3 | NDNNDNNND | 25D6 | DNNDNDDDD |
| 27K5 | KNNNKNKKK | 3D3 | NNDNNNDND | 26D6 | DNDDDNNDD |
| 28K5 | KNKNKKNNK | 4D3 | NNNDNDNND | 27D6 | DNDDDNDND |
| 29K5 | KNNKKKNNK | 5D3 | NNDNDNNND | 28D6 | DNDNDNDDD |
| 30K5 | KNNNKKNKK | 6D3 | NNNDDNNND | 29D6 | DNNDDNDDD |
| 31K5 | KNNNKKKNK | 1D4 | DDNNNDNND | 30D6 | DNDDDDNND |
| 32K5 | NKKKNNNKK | 2D4 | DDNNDNNND | 31D6 | DNDNDDNDD |
| 33K5 | NKKKNNKNK | 3D4 | DNNDNNNDD | 32D6 | DNNDDDNDD |
| 34K5 | NKKNNNKKK | 4D4 | DNDNNNDND | 33D6 | DNDNDDDND |
| 35K5 | NKNKNNKKK | 5D4 | DNNDNNDND | 34D6 | DNNDDDDND |
| 36K5 | NKKKNKNNK | 6D4 | DNDNNDNND | 35D6 | DNNNDDDDD |
| 37K5 | NKKNNKNKK | 7D4 | DNNDNDNND | 36D6 | NDDDNNDDD |
| 38K5 | NKNKNKNKK | 8D4 | DNDNDNNND | 37D6 | NDDDNDNDD |
| 39K5 | NKKNNKKNK | 9D4 | DNNDDNNND | 38D6 | NDDDNDDND |
| 40K5 | NKNKNKKNK | 10D4 | DNNNDNNDD | 39D6 | NDDNNDDDD |
| 41K5 | NKNNNKKKK | 11D4 | DNNNDNDND | 40D6 | NDNDNDDDD |
| 42K5 | NKKKKNNNK | 12D4 | DNNNDDNND | 41D6 | NDDDDNNDD |
| 43K5 | NKKNKNNKK | 13D4 | NDNDNNNDD | 42D6 | NDDDDNDND |
| 44K5 | NKNKKNNKK | 14D4 | NDDNNNDND | 43D6 | NDDNDNDDD |
| 45K5 | NKKNKNKNK | 15D4 | NDNDNDNND | 44D6 | NDNDDNDDD |
| 46K5 | NKNKKNKNK | 16D4 | NDNNNDNDD | 45D6 | NDDDDDNND |
| 47K5 | NKNNKNKKK | 17D4 | NDDNDNNND | 46D6 | NDDNDDNDD |
| 48K5 | NKKNKKNNK | 18D4 | NDNDDNNND | 47D6 | NDNDDDNDD |
| 49K5 | NKNKKKNNK | 19D4 | NDNNDNNDD | 48D6 | NDDNDDDND |
| 50K5 | NKNNKKNKK | 20D4 | NDNNDNDND | 49D6 | NDNDDDDND |
| 51K5 | NKNNKKKNK | 21D4 | NDNNDDNND | 50D6 | NDNNDDDDD |
| 52K5 | NNKKNNKKK | 22D4 | NNDDNNDND | 51D6 | NNDDNDDDD |
| 53K5 | NNKKNKNKK | 23D4 | NNDNNNDDD | 52D6 | NNDDDNDDD |
| 54K5 | NNKKNKKNK | 24D4 | NNDDNDNND | 53D6 | NNDDDDNDD |
| 55K5 | NNKNNKKKK | 25D4 | NNNDNDNDD | 54D6 | NNDDDDDND |
| 56K5 | NNNKNKKKK | 26D4 | NNDNNDDND | 55D6 | NNDNDDDDD |
| 57K5 | NNKKKNNKK | 27D4 | NNNDNDDND | 56D6 | NNNDDDDDD |
| 58K5 | NNKKKNKNK | 28D4 | NNDDDNNND | 1D7 | DDDDNNDDD |
| 59K5 | NNKNKNKKK | 29D4 | NNDNDNNDD | 2D7 | DDDDNDNDD |
| 60K5 | NNNKKNKKK | 30D4 | NNNDDNNDD | 3D7 | DDDDNDDND |
| 61K5 | NNKKKKNNK | 31D4 | NNDNDNDND | 4D7 | DDDNNDDDD |
| 62K5 | NNKNKKNKK | 32D4 | NNNDDNDND | 5D7 | DDNDNDDDD |
| 63K5 | NNNKKKNKK | 33D4 | NNDNDDNND | 6D7 | DDDDDNNDD |
| 64K5 | NNKNKKKNK | 34D4 | NNNDDDNND | 7D7 | DDDDDNDND |
| 65K5 | NNNKKKKNK | 1D5 | DDNDNNNDD | 8D7 | DDDNDNDDD |
| 1K6 | KKKKNNNKK | 2D5 | DDDNNNDND | 9D7 | DDNDDNDDD |
| 2K6 | KKKKNNKNK | 3D5 | DDNDNNDND | 10D7 | DDDDDDNND |
| 3K6 | KKKNNNKKK | 4D5 | DDDNNDNND | 11D7 | DDDNDDNDD |
| 4K6 | KKNKNNKKK | 5D5 | DDNDNDNND | 12D7 | DDNDDDNDD |
| 5K6 | KKKKNKNNK | 6D5 | DDNNNDNDD | 13D7 | DDDNDDDND |
| 6K6 | KKKNNKNKK | 7D5 | DDNNNDDND | 14D7 | DDNDDDDND |
| 7K6 | KKNKNKNKK | 8D5 | DDDNDNNND | 15D7 | DDNNDDDDD |
| 8K6 | KKKNNKKNK | 9D5 | DDNDDNNND | 16D7 | DNDDNDDDD |
| 9K6 | KKNKNKKNK | 10D5 | DDNNDNNDD | 17D7 | DNDDDNDDD |
| 10K6 | KKNNNKKKK | 11D5 | DDNNDNDND | 18D7 | DNDDDDNDD |
| 11K6 | KKKKKNNNK | 12D5 | DDNNDDNND | 19D7 | DNDDDDDND |
| 12K6 | KKKNKNNKK | 13D5 | DNDDNNNDD | 20D7 | DNDNDDDDD |
| 13K6 | KKNKKNNKK | 14D5 | DNDDNNDND | 21D7 | DNNDDDDDD |
| 14K6 | KKKNKNKNK | 15D5 | DNDNNNDDD | 22D7 | NDDDNDDDD |
| 15K6 | KKNKKNKNK | 16D5 | DNNDNNDDD | 23D7 | NDDDDNDDD |
| 16K6 | KKNNKNKKK | 17D5 | DNDDNDNND | 24D7 | NDDDDDNDD |
| 17K6 | KKKNKKNNK | 18D5 | DNDNNDNDD | 25D7 | NDDDDDDND |
| 18K6 | KKNKKKNNK | 19D5 | DNNDNDNDD | 26D7 | NDDNDDDDD |
| 19K6 | KKNNKKNKK | 20D5 | DNDNNDDND | 27D7 | NDNDDDDDD |
| 20K6 | KKNNKKKNK | 21D5 | DNNDNDDND | 28D7 | NNDDDDDDD |
| 21K6 | KNKKNNKKK | 22D5 | DNDDDNNND | 1D8 | DDDDDNDDD |
| 22K6 | KNKKNKNKK | 23D5 | DNDNDNNDD | 2D8 | DDDDDDNDD |
| 23K6 | KNKKNKKNK | 24D5 | DNNDDNNDD | 1D9 | DDDDDDDDD |

### Example 3. Identification of the Five Best Interrupted N Library Synthesis Primers

The 384 interrupted N sequences were used to generate 384 library synthesis primers. Each primer comprised a constant 5′ universal sequence (5′-GTGGTGTGTTGGGTGTGTTTGG-3′; SEQ ID NO:28) and one of the 9-mer interrupted N sequences listed in Table 4. The primers were screened by using them in whole transcriptome amplifications (WTA). The WTA screening process was performed in three steps: 1) library synthesis, 2) library amplification, and 3) gene specific qPCR.
(a) Library Synthesis and Amplification

Each library synthesis reaction comprised 2.5 μl of 1.66 ng/μl total RNA (liver) and 2.5 μl of 5 μM of one of the 384 library synthesis primers. The mixture was heated to 70° C. for 5 minutes, and then cooled on ice. To each reaction mixture, 2.5 μl of the library master mix was added (the master mix contained 1.5 mM dNTPs, 3× MMLV reaction buffer, 24 Units/μl of MMLV reverse transcriptase, and 1.2 Units/μl of Klenow exo-minus DNA polymerase, as described above). The reaction was mixed and incubated at 18° C. for 10 minutes, 25° C. for 10 minutes, 37° C. for 30 minutes, 42° C. for 10 minutes, 95° C. for 5 minutes, and then stored at 4° C. until dilution.

Each library reaction product was diluted by adding 70 μl of H₂O. The library was amplified by mixing 10 μl of diluted library and 10 μl of 2× amplification mix (2× SYBR® Green JUMPSTART™ Taq READYMIX™ and 5 μM of universal primer, 5′-GTGGTGTGTTGGGTGTGTTTGG-3′; SEQ ID NO:28). The WTA mixture was subjected to 25 cycles of 94° C. for 30 seconds and 70° C. for 5 minutes.
(b) qPCR Reactions

Each WTA product was diluted with 180 μl of H₂O and subjected to a series of “culling” qPCRs, as outline below in Table 5. The gene-specific primers used in these qPCR reactions are listed in Table 6. Each reaction mixture contained 10 μl of diluted WTA product library and 10 μl of 2× amplification mix (2× SYBR® Green JUMPSTART™ Taq READYMIX™ and 0.5 μM of each gene-specific primer). The mixture was heated to 94° C. for 2 minutes and then 40 cycles of 94° C. for 30 seconds, 60° C. for 30 seconds, and 72° C. for 30 seconds. The plates were read at 72, 76, 80, and 84° C. (MJ Opticom Monitor 2 thermocycler; MJ Research, Waltham, MA). The Ct value, which represents the PCR cycle during which the fluorescence exceeded a defined threshold level, was determined for each reaction.

TABLE 5
Screening Strategy.

| | No. of | |
|---|---|---|
| Screen | Reactions | Gene |
| 1 | 384 | beta actin |
| 2 | 96 | NM_001799 |
| 3a | 48 | NM_001570-[22348]-01 |
| 3b | 48 | Human B2M Reference Gene |
| 4a | 16 | ATP6V1G1 |
| 4b | 16 | CTNNB1 |
| 4c | 16 | GAPDH |
| 4d | 16 | GPI |
| 4e | 16 | NM_000942 |
| 4f | 16 | NM_003234 |

TABLE 6
Sequences of Gene-Specific PCR Primers.

| | Primer 1 | SEQ | Primer 2 | SEQ |
|---|---|---|---|---|
| Gene | (5′-3′) | ID NO: | (5′-3′) | ID NO: |
| beta actin | CTGGAACGGT | 29 | AAGGGACTTC | 30 |
| | GAAGGTGACA | | CTGTAACAAT | |
| | | | GCA | |
| NM_001799 | CTCAGTTGGT | 31 | TAGCAGAGTT | 32 |
| | GTGCCCAAAG | | ACTTCTAAGG | |
| | TTTCA | | GTTC | |
| NM_001570- | GATCATCCTG | 33 | GCCTTTCTTA | 34 |
| [22348]-01 | AACTGGAAAC | | CAGAAGCTGC | |
| | C | | CAAA | |
| Human | CGGCATCTTC | 35 | GCCTGCCGTG | 36 |
| B2M Ref. | AAACCTCCAT | | TGAACCATGT | |
| Gene | GA | | GACTTTGTC | |
| ATP6V1G1 | TGGACAACCT | 37 | TAAAATGCCA | 38 |
| | CTTGGCTTTT | | CTCCACAGCA | |
| CTNNB1 | TTGAAAATCC | 39 | TCGAGTCATT | 40 |
| | AGCGTGGACA | | GCATACTGTC | |
| GAPDH | GAAGGTGAAG | 41 | GAAGATGGTG | 41 |
| | GTCGGAGTC | | ATGGGATTTC | |
| GPI | AGGCTGCTGC | 43 | CCAAGGCTCC | 44 |
| | CACATAAGGT | | AAGCATGAAT | |
| NM_000942 | CAAAGTCACC | 45 | GGAACAGTCT | 46 |
| | GTCAAGGTGT | | TTCCGAAGAG | |
| | AT | | ACCAA | |
| NM_003234 | CAGACTAACA | 47 | GAGGAAGTGA | 48 |
| | ACAGATTTCG | | TACTCCACTC | |
| | GGAAT | | TCAT | |

The first qPCR screen comprised amplification of the beta actin gene. The reactions were performed in four 96-well plates. To mitigate plate-to-plate variation, each plate's average Ct was calculated and the delta Ct (ΔCt) of each reaction on a plate was determined as Ct(avg)-Ct(reaction). Data from the four qPCR plates were combined into a single table and sorted on delta Ct (Table 7). Inspection of the table revealed no apparent plate biasing (i.e. the distribution of delta Cts appeared statistically distributed between the four plates).

TABLE 7
First qPCR Screen - Amplification of Beta Actin.

| | DNA | Sequence | Ct | delta |
|---|---|---|---|---|
| Plate | name | (5′-3′) | (dR) | Ct |
| 1 | 8D7 | DDDNDNDDD | NoCt | NA |
| 2 | 16D7 | DNDDNDDDD | 10.33 | 3.14 |
| 3 | 1D9 | DDDDDDDDD | 10.92 | 2.23 |
| 2 | 13K6 | KKNKKNNKK | 11.66 | 1.81 |
| 2 | 19D7 | DNDDDDDND | 11.81 | 1.66 |
| 2 | 45K6 | NKKKKKNNK | 11.94 | 1.53 |
| 2 | 17D7 | DNDDDNDDD | 12.02 | 1.45 |
| 3 | 24D7 | NDDDDDNDD | 11.8 | 1.35 |
| 2 | 18K4 | NKNKKNNNK | 12.15 | 1.32 |
| 1 | 2K5 | KKKNNNKNK | 15.22 | 1.32 |
| 4 | 56K6 | NNNKKKKKK | 14.82 | 1.31 |
| 3 | 54D6 | NNDDDDDND | 11.9 | 1.25 |
| 3 | 25D7 | NDDDDDDND | 11.91 | 1.24 |
| 2 | 40D6 | NDNDNDDDD | 12.26 | 1.21 |
| 2 | 18D7 | DNDDDDNDD | 12.28 | 1.19 |
| 3 | 27D7 | NDNDDDDDD | 11.96 | 1.19 |
| 2 | 8K6 | KKKNNKKNK | 12.28 | 1.19 |
| 3 | 54D5 | NNDDNDDND | 12.01 | 1.14 |
| 4 | 60K5 | NNNKKNKKK | 15 | 1.13 |
| 4 | 29K6 | KNNKKNKKK | 15.02 | 1.11 |
| 2 | 11K4 | KNNNKNKNK | 12.41 | 1.06 |
| 2 | 14K6 | KKKNKNKNK | 12.42 | 1.05 |
| 2 | 20D7 | DNDNDDDDD | 12.44 | 1.03 |
| 4 | 3K7 | KKKKNKKNK | 15.11 | 1.02 |
| 2 | 8D6 | DDDNNDDND | 12.48 | 0.99 |
| 4 | 27K4 | NNNKNKKNK | 15.15 | 0.98 |
| 4 | 32K4 | NNNKKNKNK | 15.18 | 0.95 |
| 4 | 31D5 | DNNNDDDND | 15.2 | 0.93 |
| 1 | 38K5 | NKNKNKNKK | 15.63 | 0.9 |
| 3 | 1D8 | DDDDDNDDD | 12.27 | 0.88 |
| 1 | 34K5 | NKKNNNKKK | 15.66 | 0.88 |
| 2 | 9D5 | DDNDDNNND | 12.61 | 0.86 |
| 2 | 14D6 | DDDNDNDND | 12.61 | 0.86 |
| 1 | 65D5 | NNNDDDDND | 15.69 | 0.85 |
| 1 | 35K6 | KNNNKKKKK | 15.69 | 0.85 |
| 3 | 24K4 | NNKKNKNNK | 12.32 | 0.83 |
| 3 | 19K4 | NKNNKNNKK | 12.34 | 0.81 |
| 2 | 48D5 | NDDNDDNND | 12.67 | 0.8 |
| 3 | 28D7 | NNDDDDDDD | 12.35 | 0.8 |
| 3 | 1K9 | KKKKKKKKK | 12.35 | 0.8 |
| 1 | 5K6 | KKKKNKNNK | 15.75 | 0.79 |
| 1 | 5D6 | DDDDNDNND | 15.76 | 0.78 |
| 2 | 14K4 | NKKNNNKNK | 12.7 | 0.77 |
| 2 | 15K4 | NKNKNKNNK | 12.7 | 0.77 |
| 2 | 41D5 | NDNNNDDDD | 12.7 | 0.77 |
| 1 | 8K5 | KKKNKNNNK | 15.77 | 0.77 |
| 2 | 12K4 | KNNNKKNNK | 12.72 | 0.75 |
| 1 | 36K5 | NKKKNKNNK | 15.79 | 0.75 |
| 1 | 9K7 | KKNKKNKKK | 15.79 | 0.75 |
| 4 | 4K7 | KKKNNKKKK | 15.38 | 0.75 |
| 2 | 48K5 | NKKNKKNNK | 12.73 | 0.74 |
| 4 | 6K3 | NNNKKNNNK | 15.39 | 0.74 |
| 4 | 4K3 | NNNKNKNNK | 15.41 | 0.72 |
| 4 | 57K5 | NNKKKNNKK | 15.42 | 0.71 |
| 1 | 6K5 | KKNNNKNKK | 15.84 | 0.7 |
| 2 | 13K4 | NKNKNNNKK | 12.78 | 0.69 |
| 3 | 24K5 | KNNKKNNKK | 12.46 | 0.69 |
| 3 | 49D5 | NDNDDDNND | 12.49 | 0.66 |
| 2 | 16K5 | KNNKNNKKK | 12.81 | 0.66 |
| 4 | 62K5 | NNKNKKNKK | 15.47 | 0.66 |
| 2 | 9K5 | KKNKKNNNK | 12.84 | 0.63 |
| 3 | 24K7 | NKKKKKNKK | 12.52 | 0.63 |
| 1 | 15K7 | KKNNKKKKK | 15.91 | 0.63 |
| 3 | 16D6 | DDNNDNDDD | 12.53 | 0.62 |
| 4 | 6K7 | KKKKKNNKK | 15.51 | 0.62 |
| 2 | 12D6 | DDDNDNNDD | 12.86 | 0.61 |
| 2 | 22D7 | NDDDNDDDD | 12.86 | 0.61 |
| 2 | 21K7 | KNNKKKKKK | 12.86 | 0.61 |
| 4 | 5D7 | DDNDNDDDD | 15.52 | 0.61 |
| 4 | 7D7 | DDDDDNDND | 15.53 | 0.6 |
| 4 | 5K7 | KKNKNKKKK | 15.53 | 0.6 |
| 4 | 61K5 | NNKKKKNNK | 15.54 | 0.59 |
| 2 | 16K7 | KNKKNKKKK | 12.88 | 0.59 |
| 3 | 25K4 | NNNKNKNKK | 12.58 | 0.57 |
| 1 | 13D7 | DDDNDDDND | 15.97 | 0.57 |
| 3 | 26D7 | NDDNDDDDD | 12.59 | 0.56 |
| 1 | 11K7 | KKKNKKNKK | 15.98 | 0.56 |
| 4 | 30K5 | KNNNKKNKK | 15.58 | 0.55 |
| 4 | 27D5 | DNNNDNDDD | 15.59 | 0.54 |
| 2 | 13D5 | DNDDNNNDD | 12.94 | 0.53 |
| 4 | 30D5 | DNNNDDNDD | 15.61 | 0.52 |
| 3 | 54K5 | NNKKNKKNK | 12.63 | 0.52 |
| 4 | 63D5 | NNNDDDNDD | 15.62 | 0.51 |
| 4 | 32D4 | NNNDDNDND | 15.63 | 0.5 |
| 3 | 54K6 | NNKKKKKNK | 12.66 | 0.49 |
| 2 | 42D6 | NDDDDNDND | 13 | 0.47 |
| 3 | 48D6 | NDDNDDDND | 12.68 | 0.47 |
| 3 | 55K6 | NNKNKKKKK | 12.68 | 0.47 |
| 4 | 64D5 | NNDNDDDND | 15.67 | 0.46 |
| 4 | 30K4 | NNNKKNNKK | 15.67 | 0.46 |
| 4 | 1K7 | KKKKNNKKK | 15.67 | 0.46 |
| 3 | 21D6 | DNDDNNDDD | 12.7 | 0.45 |
| 3 | 49K5 | NKNKKKNNK | 12.7 | 0.45 |
| 2 | 18K7 | KNKKKKNKK | 13.02 | 0.45 |
| 1 | 5D5 | DDNDNDNND | 16.09 | 0.45 |
| 4 | 61D5 | NNDDDDNND | 15.68 | 0.45 |
| 4 | 28K5 | KNKNKKNNK | 15.68 | 0.45 |
| 3 | 51K6 | NNKKNKKKK | 12.71 | 0.44 |
| 4 | 2K3 | NKNNKNNNK | 15.7 | 0.43 |
| 1 | 6D5 | DDNNNDNDD | 16.11 | 0.43 |
| 2 | 41K5 | NKNNNKKKK | 13.05 | 0.42 |
| 2 | 41K6 | NKKKKNNKK | 13.05 | 0.42 |
| 4 | 2K7 | KKKKNKNKK | 15.71 | 0.42 |
| 2 | 17K4 | NKKNKNNNK | 13.06 | 0.41 |
| 3 | 53D6 | NNDDDDNDD | 12.75 | 0.4 |
| 2 | 22K7 | NKKKNKKKK | 13.07 | 0.4 |
| 4 | 28K4 | NNKKKNNNK | 15.74 | 0.39 |
| 4 | 33K4 | NNKNKKNNK | 15.74 | 0.39 |
| 4 | 63K5 | NNNKKKNKK | 15.74 | 0.39 |
| 3 | 17K5 | KNKKNKNNK | 12.77 | 0.38 |
| 4 | 29K4 | NNKNKNNKK | 15.76 | 0.37 |
| 3 | 2D8 | DDDDDDNDD | 12.79 | 0.36 |
| 4 | 59D5 | NNDNDNDDD | 15.77 | 0.36 |
| 2 | 13D6 | DDNDDNNDD | 13.12 | 0.35 |
| 1 | 4K5 | KKKNNKNNK | 16.19 | 0.35 |
| 3 | 21D5 | DNNDNDDND | 12.81 | 0.34 |
| 3 | 26K4 | NNKNNKKNK | 12.81 | 0.34 |
| 2 | 42K6 | NKKKKNKNK | 13.14 | 0.33 |
| 4 | 27K5 | KNNNKNKKK | 15.8 | 0.33 |
| 3 | 23D5 | DNDNDNNDD | 12.83 | 0.32 |
| 3 | 23K4 | NNKNNNKKK | 12.83 | 0.32 |
| 3 | 21K5 | KNNKNKKNK | 12.83 | 0.32 |
| 2 | 15K5 | KNKNNNKKK | 13.15 | 0.32 |
| 4 | 29D5 | DNNDDDNND | 15.82 | 0.31 |
| 4 | 25K6 | KNNKNKKKK | 15.82 | 0.31 |
| 3 | 50D6 | NDNNDDDDD | 12.85 | 0.3 |
| 2 | 12K6 | KKKNKNNKK | 13.17 | 0.3 |
| 4 | 26K6 | KNKKKNNKK | 15.83 | 0.3 |
| 3 | 56K5 | NNNKNKKKK | 12.86 | 0.29 |
| 1 | 35D5 | NDNDNNDDD | 16.25 | 0.29 |
| 1 | 10K4 | KNNNKNNKK | 16.25 | 0.29 |
| 1 | 34D6 | DNNDDDDND | 16.25 | 0.29 |
| 4 | 29D6 | DNNDDNDDD | 15.84 | 0.29 |
| 3 | 17D5 | DNDDNDNND | 12.88 | 0.27 |
| 3 | 26K7 | NKKNKKKKK | 12.88 | 0.27 |
| 4 | 25D5 | DNDNDNDND | 15.87 | 0.26 |
| 3 | 23D6 | DNDDNDDND | 12.89 | 0.26 |
| 3 | 22D5 | DNDDDNNND | 12.9 | 0.25 |
| 4 | 30K6 | KNKKKKNNK | 15.88 | 0.25 |
| 2 | 44D5 | NDNDDNNDD | 13.23 | 0.24 |
| 3 | 48K6 | NKKNKKKNK | 12.91 | 0.24 |
| 3 | 25K7 | NKKKKKKNK | 12.91 | 0.24 |
| 2 | 18D4 | NDNDDNNND | 13.25 | 0.22 |
| 3 | 21K4 | NKNNKKNNK | 12.94 | 0.21 |
| 3 | 50K6 | NKNNKKKKK | 12.94 | 0.21 |
| 1 | 8K4 | KNKNKNNNK | 16.34 | 0.2 |
| 2 | 11D5 | DDNNDNDND | 13.28 | 0.19 |
| 2 | 46D5 | NDNDDNDND | 13.29 | 0.18 |
| 3 | 16K6 | KKNNKNKKK | 12.97 | 0.18 |
| 2 | 14D5 | DNDDNNDND | 13.3 | 0.17 |
| 1 | 2K6 | KKKKNNKNK | 16.38 | 0.16 |
| 3 | 28K7 | NNKKKKKKK | 12.99 | 0.16 |
| 4 | 62D5 | NNDNDDNDD | 15.97 | 0.16 |
| 2 | 10D6 | DDNNNDDDD | 13.33 | 0.14 |
| 2 | 16K4 | NKNNNKNKK | 13.33 | 0.14 |
| 1 | 36D6 | NDDDNNDDD | 16.4 | 0.14 |
| 4 | 1K3 | KNNNKNNNK | 15.99 | 0.14 |
| 2 | 41D6 | NDDDDNNDD | 13.34 | 0.13 |
| 2 | 21D7 | DNNDDDDDD | 13.34 | 0.13 |
| 1 | 39K6 | NKKNNKKKK | 16.41 | 0.13 |
| 4 | 33D4 | NNDNDDNND | 16.01 | 0.12 |
| 4 | 26D5 | DNNDDNDND | 16.01 | 0.12 |
| 1 | 10K7 | KKKKKKNNK | 16.42 | 0.12 |
| 2 | 17D4 | NDDNDNNND | 13.36 | 0.11 |
| 3 | 20D6 | DDNNDDDND | 13.04 | 0.11 |
| 2 | 46D6 | NDDNDDNDD | 13.37 | 0.1 |
| 3 | 24D5 | DNNDDNNDD | 13.05 | 0.1 |
| 3 | 53K6 | NNKKKKNKK | 13.05 | 0.1 |
| 4 | 28D5 | DNDNDDNND | 16.03 | 0.1 |
| 1 | 1K6 | KKKKNNNKK | 16.44 | 0.1 |
| 4 | 28K6 | KNKNKNKKK | 16.04 | 0.09 |
| 2 | 45K5 | NKKNKNKNK | 13.39 | 0.08 |
| 1 | 4K4 | KNKNNNKNK | 16.46 | 0.08 |
| 4 | 25K5 | KNKNKNKNK | 16.05 | 0.08 |
| 4 | 3K3 | NNKNNNKNK | 16.06 | 0.07 |
| 4 | 1D4 | DDNNNDNND | 16.08 | 0.05 |
| 2 | 11D4 | DNNNDNDND | 13.42 | 0.05 |
| 3 | 20K4 | NKNNKNKNK | 13.11 | 0.04 |
| 2 | 44K5 | NKNKKNNKK | 13.43 | 0.04 |
| 2 | 47D5 | NDNNDNDDD | 13.44 | 0.03 |
| 2 | 46K5 | NKNKKNKNK | 13.44 | 0.03 |
| 1 | 36D5 | NDDDNDNND | 16.51 | 0.03 |
| 4 | 7K7 | KKKKKNKNK | 16.1 | 0.03 |
| 3 | 51D5 | NDNNDDDND | 13.13 | 0.02 |
| 4 | 24K6 | KNKNNKKKK | 16.11 | 0.02 |
| 2 | 10K6 | KKNNNKKKK | 13.46 | 0.01 |
| 2 | 44K6 | NKNKKNKKK | 13.46 | 0.01 |
| 1 | 1 D6 | DDDDNNNDD | 16.53 | 0.01 |
| 4 | 60D5 | NNNDDNDDD | 16.12 | 0.01 |
| 2 | 45D6 | NDDDDDNND | 13.47 | 0 |
| 1 | 4D6 | DDNDNNDDD | 16.54 | 0 |
| 3 | 22K4 | NNKKNNKNK | 13.15 | 0 |
| 1 | 15D7 | DDNNDDDDD | 16.54 | 0 |
| 2 | 43D6 | NDDNDNDDD | 13.48 | −0.01 |
| 2 | 43K5 | NKKNKNNKK | 13.48 | −0.01 |
| 1 | 13K7 | KKKNKKKNK | 16.55 | −0.01 |
| 1 | 5D4 | DNNDNNDND | 16.56 | −0.02 |
| 2 | 9D6 | DDNDNDDND | 13.49 | −0.02 |
| 2 | 13K5 | KNKKNNNKK | 13.5 | −0.03 |
| 1 | 9D4 | DNNDDNNND | 16.57 | −0.03 |
| 1 | 33K6 | KNKNKKKNK | 16.57 | −0.03 |
| 1 | 4D4 | DNDNNNDND | 16.58 | −0.04 |
| 1 | 7D4 | DNNDNDNND | 16.58 | −0.04 |
| 1 | 38D5 | NDNDNDNDD | 16.58 | −0.04 |
| 4 | 3D3 | NNDNNNDND | 16.17 | −0.04 |
| 4 | 59K5 | NNKNKNKKK | 16.17 | −0.04 |
| 3 | 56D5 | NNNDNDDDD | 13.2 | −0.05 |
| 3 | 55D6 | NNDNDDDDD | 13.2 | −0.05 |
| 3 | 52K6 | NNKKKNKKK | 13.2 | −0.05 |
| 1 | 37K6 | NKKKNKNKK | 16.59 | −0.05 |
| 2 | 11K5 | KKNNKNKNK | 13.53 | −0.06 |
| 2 | 19K7 | KNKKKKKNK | 13.53 | −0.06 |
| 2 | 23K7 | NKKKKNKKK | 13.53 | −0.06 |
| 3 | 27K7 | NKNKKKKKK | 13.21 | −0.06 |
| 4 | 31D4 | NNDNDNDND | 16.19 | −0.06 |
| 1 | 2D6 | DDDDNNDND | 16.6 | −0.06 |
| 2 | 10K5 | KKNNKNNKK | 13.54 | −0.07 |
| 3 | 18K6 | KKNKKKNNK | 13.22 | −0.07 |
| 4 | 27K6 | KNKKKNKNK | 16.2 | −0.07 |
| 4 | 57D5 | NNDDDNNDD | 16.21 | −0.08 |
| 1 | 37D5 | NDDNNDNDD | 16.64 | −0.1 |
| 4 | 26K5 | KNNKKNKNK | 16.24 | −0.11 |
| 2 | 23D7 | NDDDDNDDD | 13.59 | −0.12 |
| 2 | 47K5 | NKNNKNKKK | 13.6 | −0.13 |
| 2 | 12D5 | DDNNDDNND | 13.61 | −0.14 |
| 3 | 22D6 | DNDDNDNDD | 13.29 | −0.14 |
| 3 | 24D4 | NNDDNDNND | 13.3 | −0.15 |
| 1 | 6D6 | DDDNNDNDD | 16.69 | −0.15 |
| 1 | 7K6 | KKNKNKNKK | 16.69 | −0.15 |
| 4 | 34D4 | NNNDDDNND | 16.29 | −0.16 |
| 4 | 1K4 | KKNNNKNNK | 16.29 | −0.16 |
| 3 | 51K5 | NKNNKKKNK | 13.31 | −0.16 |
| 3 | 21K6 | KNKKNNKKK | 13.32 | −0.17 |
| 4 | 2D3 | NDNNDNNND | 16.3 | −0.17 |
| 4 | 5D3 | NNDNDNNND | 16.3 | −0.17 |
| 4 | 31K5 | KNNNKKKNK | 16.3 | −0.17 |
| 1 | 34K6 | KNNKKKKNK | 16.72 | −0.18 |
| 4 | 58K5 | NNKKKNKNK | 16.31 | −0.18 |
| 3 | 55D5 | NNDNNDDDD | 13.34 | −0.19 |
| 1 | 14D7 | DDNDDDDND | 16.73 | −0.19 |
| 4 | 4D7 | DDDNNDDDD | 16.32 | −0.19 |
| 2 | 10D5 | DDNNDNNDD | 13.67 | −0.2 |
| 2 | 40K6 | NKNKNKKKK | 13.67 | −0.2 |
| 3 | 19D4 | NDNNDNNDD | 13.36 | −0.21 |
| 1 | 34D5 | NDDNNNDDD | 16.75 | −0.21 |
| 1 | 3K6 | KKKNNNKKK | 16.76 | −0.22 |
| 2 | 15D4 | NDNDNDNND | 13.7 | −0.23 |
| 3 | 49K6 | NKNKKKKNK | 13.38 | −0.23 |
| 1 | 38D6 | NDDDNDDND | 16.78 | −0.24 |
| 4 | 5K3 | NNKNKNNNK | 16.38 | −0.25 |
| 1 | 7D6 | DDNDNDNDD | 16.79 | −0.25 |
| 1 | 8D4 | DNDNDNNND | 16.81 | −0.27 |
| 1 | 7K4 | KNNKNKNNK | 16.81 | −0.27 |
| 4 | 4D3 | NNNDNDNND | 16.4 | −0.27 |
| 2 | 43D5 | NDDNDNNDD | 13.74 | −0.27 |
| 3 | 2K8 | KKKKKKNKK | 13.42 | −0.27 |
| 4 | 29D4 | NNDNDNNDD | 16.41 | −0.28 |
| 1 | 6K4 | KNKNNKNNK | 16.82 | −0.28 |
| 2 | 14D4 | NDDNNNDND | 13.76 | −0.29 |
| 1 | 33D5 | NDDDNNDND | 16.83 | −0.29 |
| 1 | 33D6 | DNDNDDDND | 16.83 | −0.29 |
| 2 | 13D4 | NDNDNNNDD | 13.77 | −0.3 |
| 3 | 53K5 | NNKKNKNKK | 13.45 | −0.3 |
| 1 | 5K5 | KKNKNKNNK | 16.84 | −0.3 |
| 1 | 7K5 | KKNNNKKNK | 16.84 | −0.3 |
| 1 | 6K6 | KKKNNKNKK | 16.84 | −0.3 |
| 2 | 42D5 | NDDDDNNND | 13.78 | −0.31 |
| 2 | 15K6 | KKNKKNKNK | 13.78 | −0.31 |
| 3 | 55K5 | NNKNNKKKK | 13.47 | −0.32 |
| 1 | 3K5 | KKNKNNKNK | 16.86 | −0.32 |
| 2 | 11D6 | DDDDDNNND | 13.8 | −0.33 |
| 3 | 19D5 | DNNDNDNDD | 13.48 | −0.33 |
| 3 | 53D5 | NNDDNDNDD | 13.48 | −0.33 |
| 1 | 39D6 | NDDNNDDDD | 16.87 | −0.33 |
| 1 | 37K5 | NKKNNKNKK | 16.87 | −0.33 |
| 3 | 23D4 | NNDNNNDDD | 13.5 | −0.35 |
| 1 | 65K5 | NNNKKKKNK | 16.89 | −0.35 |
| 1 | 2D5 | DDDNNNDND | 16.9 | −0.36 |
| 1 | 4K6 | KKNKNNKKK | 16.9 | −0.36 |
| 3 | 1K8 | KKKKKNKKK | 13.51 | −0.36 |
| 2 | 16D4 | NDNNNDNDD | 13.84 | −0.37 |
| 3 | 18K5 | KNKNNKNKK | 13.52 | −0.37 |
| 1 | 8D5 | DDDNDNNND | 16.92 | −0.38 |
| 1 | 32D6 | DNNDDDNDD | 16.92 | −0.38 |
| 4 | 2D7 | DDDDNDNDD | 16.51 | −0.38 |
| 2 | 44D6 | NDNDDNDDD | 13.86 | −0.39 |
| 4 | 25D6 | DNNDNDDDD | 16.53 | −0.4 |
| 3 | 26D4 | NNDNNDDND | 13.56 | −0.41 |
| 4 | 30D6 | DNDDDDNND | 16.54 | −0.41 |
| 2 | 20K7 | KNKNKKKKK | 13.88 | −0.41 |
| 1 | 7D5 | DDNNNDDND | 16.95 | −0.41 |
| 3 | 18D6 | DDNDDDNND | 13.57 | −0.42 |
| 3 | 50K5 | NKNNKKNKK | 13.57 | −0.42 |
| 4 | 27D4 | NNNDNDDND | 16.55 | −0.42 |
| 2 | 46K6 | NKKNKKNKK | 13.91 | −0.44 |
| 4 | 6D7 | DDDDDNNDD | 16.57 | −0.44 |
| 1 | 8K7 | KKKNKNKKK | 16.99 | −0.45 |
| 3 | 20K6 | KKNNKKKNK | 13.62 | −0.47 |
| 3 | 22K6 | KNKKNKNKK | 13.64 | −0.49 |
| 1 | 36K6 | NKKKNNKKK | 17.03 | −0.49 |
| 2 | 11K6 | KKKKKNNNK | 13.97 | −0.5 |
| 3 | 49D6 | NDNDDDDND | 13.66 | −0.51 |
| 1 | 1D5 | DDNDNNNDD | 17.06 | −0.52 |
| 4 | 28D4 | NNDDDNNND | 16.65 | −0.52 |
| 3 | 21D4 | NDNNDDNND | 13.67 | −0.52 |
| 3 | 25D4 | NNNDNDNDD | 13.68 | −0.53 |
| 3 | 17K6 | KKKNKKNNK | 13.68 | −0.53 |
| 2 | 9K6 | KKNKNKKNK | 14 | −0.53 |
| 4 | 58D5 | NNDDDNDND | 16.66 | −0.53 |
| 1 | 3D4 | DNNDNNNDD | 17.08 | −0.54 |
| 3 | 20K5 | KNKNNKKNK | 13.7 | −0.55 |
| 1 | 40K5 | NKNKNKKNK | 17.09 | −0.55 |
| 1 | 3D6 | DDDNNNDDD | 17.1 | −0.56 |
| 4 | 28D6 | DNDNDNDDD | 16.69 | −0.56 |
| 1 | 38K6 | NKKKNKKNK | 17.11 | −0.57 |
| 1 | 39K5 | NKKNNKKNK | 17.12 | −0.58 |
| 4 | 34K4 | NNNKKKNNK | 16.73 | −0.6 |
| 4 | 30D4 | NNNDDNNDD | 16.74 | −0.61 |
| 1 | 40D5 | NDNDNDDND | 17.15 | −0.61 |
| 3 | 23K5 | KNKNKNNKK | 13.77 | −0.62 |
| 1 | 35K5 | NKNKNNKKK | 17.17 | −0.63 |
| 1 | 12K7 | KKNKKKNKK | 17.18 | −0.64 |
| 4 | 31K4 | NNKNKNKNK | 16.78 | −0.65 |
| 4 | 6D3 | NNNDDNNND | 16.79 | −0.66 |
| 1 | 3D5 | DDNDNNDND | 17.2 | −0.66 |
| 3 | 50D5 | NDNNDDNDD | 13.82 | −0.67 |
| 3 | 23K6 | KNKKNKKNK | 13.82 | −0.67 |
| 1 | 6D4 | DNDNNDNND | 17.21 | −0.67 |
| 1 | 14K7 | KKNKKKKNK | 17.21 | −0.67 |
| 1 | 37D6 | NDDDNDNDD | 17.22 | −0.68 |
| 3 | 19K5 | KNNKNKNKK | 13.83 | −0.68 |
| 2 | 12D4 | DNNNDDNND | 14.16 | −0.69 |
| 2 | 14K5 | KNKKNNKNK | 14.16 | −0.69 |
| 1 | 32K6 | KNNKKKNKK | 17.23 | −0.69 |
| 4 | 32K5 | NKKKNNNKK | 16.83 | −0.7 |
| 4 | 64K5 | NNKNKKKNK | 16.83 | −0.7 |
| 2 | 45D5 | NDDNDNDND | 14.18 | −0.71 |
| 4 | 26D6 | DNDDDNNDD | 16.84 | −0.71 |
| 4 | 3D7 | DDDDNDDND | 16.84 | −0.71 |
| 1 | 10D4 | DNNNDNNDD | 17.26 | −0.72 |
| 4 | 2D4 | DDNNDNNND | 16.86 | −0.73 |
| 1 | 11D7 | DDDNDDNDD | 17.27 | −0.73 |
| 4 | 1D3 | DNNNDNNND | 16.95 | −0.82 |
| 3 | 19K6 | KKNNKKNKK | 13.97 | −0.82 |
| 2 | 12K5 | KKNNKKNNK | 14.34 | −0.87 |
| 3 | 52D5 | NNDDNNDDD | 14.03 | −0.88 |
| 2 | 15D5 | DNDNNNDDD | 14.36 | −0.89 |
| 2 | 43K6 | NKKNKNKKK | 14.4 | −0.93 |
| 3 | 20D4 | NDNNDNDND | 14.09 | −0.94 |
| 2 | 47K6 | NKNKKKNKK | 14.41 | −0.94 |
| 3 | 18D5 | DNDNNDNDD | 14.11 | −0.96 |
| 1 | 12D7 | DDNDDDNDD | 17.5 | −0.96 |
| 1 | 35D6 | DNNNDDDDD | 17.51 | −0.97 |
| 3 | 22D4 | NNDDNNDND | 14.14 | −0.99 |
| 4 | 31D6 | DNDNDDNDD | 17.12 | −0.99 |
| 1 | 33K5 | NKKKNNKNK | 17.54 | −1 |
| 4 | 32D5 | NDDDNNNDD | 17.14 | −1.01 |
| 3 | 19D6 | DDNNDDNDD | 14.16 | −1.01 |
| 2 | 17K7 | KNKKKNKKK | 14.48 | −1.01 |
| 4 | 2K4 | KKNNKNNNK | 17.18 | −1.05 |
| 3 | 52K5 | NNKKNNKKK | 14.22 | −1.07 |
| 1 | 5K4 | KNNKNNKNK | 17.63 | −1.09 |
| 4 | 31K6 | KNKNKKNKK | 17.23 | −1.1 |
| 4 | 24D6 | DNDNNDDDD | 17.24 | −1.11 |
| 1 | 1K5 | KKNKNNNKK | 17.67 | −1.13 |
| 1 | 9K4 | KNNKKNNNK | 17.68 | −1.14 |
| 1 | 10D7 | DDDDDDNND | 17.68 | −1.14 |
| 1 | 4D5 | DDDNNDNND | 17.69 | −1.15 |
| 3 | 22K5 | KNKKKNNNK | 14.35 | −1.2 |
| 4 | 1D7 | DDDDNNDDD | 17.34 | −1.21 |
| 1 | 3K4 | KNNKNNNKK | 17.76 | −1.22 |
| 4 | 27D6 | DNDDDNDND | 17.41 | −1.28 |
| 3 | 17D6 | DDDNDDNND | 14.46 | −1.31 |
| 1 | 39D5 | NDDNNDDND | 17.9 | −1.36 |
| 1 | 9D7 | DDNDDNDDD | 17.95 | −1.41 |
| 3 | 20D5 | DNDNNDDND | 14.7 | −1.55 |
| 4 | 29K5 | KNNKKKNNK | 17.68 | −1.55 |
| 3 | 52D6 | NNDDDNDDD | 14.84 | −1.69 |
| 3 | 51D6 | NNDDNDDDD | 14.96 | −1.81 |
| 4 | 56D6 | NNNDDDDDD | 18.51 | −2.38 |
| 2 | 16D5 | DNNDNNDDD | 16.85 | −3.38 |
| 2 | 47D6 | NDNDDDNDD | 17.38 | −3.91 |
| 2 | 15D6 | DDNDDNDND | 19.11 | −5.64 |
| 2 | 42K5 | NKKKKNNNK | 24.63 | −11.16 |

The top 96 WTA products (underlined in Table 7) were then subjected to a second qPCR screen using primers for NM_001799 in a single plate. Table 8 presents the efficiency of amplification and Ct value for each reaction. The WTA products were ranked from lowest Ct to highest Ct.

TABLE 8
Second qPCR Screen-Amplification of NM_001799.

| DNA | Sequence | | Ct |
|---|---|---|---|
| name | (5′-3′) | Efficiency | (dR) |
| 1K9 | KKKKKKKKK | 80.08% | 17.31 |
| 54D5 | NNDDNDDND | 57.92% | 17.54 |
| 32D4 | NNNDDNDND | 85.97% | 18.17 |
| 61D5 | NNDDDDNND | 79.33% | 18.49 |
| 34K5 | NKKNNNKKK | 46.90% | 18.62 |
| 1D8 | DDDDDNDDD | 96.17% | 18.82 |
| 6K7 | KKKKKNNKK | 69.67% | 18.84 |
| 1D9 | DDDDDDDDD | 83.07% | 19.03 |
| 5K6 | KKKKNKNNK | 84.51% | 19.05 |
| 24D7 | NDDDDDNDD | 72.39% | 19.12 |
| 61K5 | NNKKKKNNK | 80.13% | 19.52 |
| 13D7 | DDDNDDDND | 81.62% | 19.54 |
| 25D7 | NDDDDDDND | 88.34% | 19.65 |
| 30K4 | NNNKKNNKK | 90.85% | 19.72 |
| 24K5 | KNNKKNNKK | 83.17% | 19.73 |
| 54D6 | NNDDDDDND | 90.44% | 19.86 |
| 65D5 | NNNDDDDND | 62.60% | 19.98 |
| 30D5 | DNNNDDNDD | 94.49% | 20.1 |
| 4K7 | KKKNNKKKK | 75.22% | 20.13 |
| 36K5 | NKKKNKNNK | 93.89% | 20.21 |
| 27K4 | NNNKNKKNK | 89.10% | 20.26 |
| 24K4 | NNKKNKNNK | 73.40% | 20.27 |
| 54K5 | NNKKNKKNK | 86.13% | 20.43 |
| 12K4 | KNNNKKNNK | 104.05% | 20.47 |
| 8K6 | KKKNNKKNK | 100.82% | 20.61 |
| 4K3 | NNNKNKNNK | 87.94% | 20.64 |
| 25K4 | NNNKNKNKK | 70.83% | 20.75 |
| 54K6 | NNKKKKKNK | 81.43% | 20.85 |
| 41D5 | NDNNNDDDD | 93.97% | 20.93 |
| 15K4 | NKNKNKNNK | 77.28% | 20.97 |
| 9D5 | DDNDDNNND | 85.41% | 21 |
| 27D5 | DNNNDNDDD | 70.42% | 21.15 |
| 24K7 | NKKKKKNKK | 74.31% | 21.16 |
| 11K4 | KNNNKNKNK | 100.68% | 21.36 |
| 18K4 | NKNKKNNNK | 87.46% | 21.5 |
| 38K5 | NKNKNKNKK | 60.88% | 21.89 |
| 31D5 | DNNNDDDND | 85.38% | 21.92 |
| 19D7 | DNDDDDDND | 85.09% | 22.12 |
| 16D7 | DNDDNDDDD | 86.72% | 22.31 |
| 16K7 | KNKKNKKKK | 93.38% | 22.44 |
| 21D6 | DNDDNNDDD | 84.90% | 22.69 |
| 3K7 | KKKKNKKNK | 72.22% | 22.78 |
| 55K6 | NNKNKKKKK | 92.61% | 22.97 |
| 19K4 | NKNNKNNKK | 76.80% | 23.6 |
| 18D7 | DNDDDDNDD | 95.54% | 24.73 |
| 16K5 | KNNKNNKKK | 85.72% | 25.04 |
| 22D7 | NDDDNDDDD | 79.40% | 25.32 |
| 13K6 | KKNKKNNKK | 69.91% | 27.65 |
| 9K7 | KKNKKNKKK | 21.93% | 30.7 |
| 14K4 | NKKNNNKNK | 53.68% | 31.19 |
| 21K7 | KNNKKKKKK | 22.03% | 31.2 |
| 2K5 | KKKNNNKNK | 35.00% | 32.12 |
| 62K5 | NNKNKKNKK | 11.41% | 32.14 |
| 9K5 | KKNKKNNNK | 46.20% | 32.16 |
| 17D7 | DNDDDNDDD | 38.26% | 32.43 |
| 18K7 | KNKKKKNKK | 47.58% | 32.61 |
| 5D5 | DDNDNDNND | 40.41% | 32.74 |
| 27D7 | NDNDDDDDD | 45.11% | 32.76 |
| 11K7 | KKKNKKNKK | 45.26% | 33.14 |
| 57K5 | NNKKKNNKK | 54.33% | 33.28 |
| 49K5 | NKNKKKNNK | 7.11% | 33.49 |
| 35K6 | KNNNKKKKK | 44.63% | 33.51 |
| 49D5 | NDNDDDNND | 40.46% | 33.67 |
| 12D6 | DDDNDNNDD | 50.40% | 33.96 |
| 8D6 | DDDNNDDND | 63.04% | 34.12 |
| 7D7 | DDDDDNDND | 43.04% | 34.12 |
| 64D5 | NNDNDDDND | 56.63% | 34.14 |
| 6K5 | KKNNNKNKK | 59.05% | 34.19 |
| 5K7 | KKNKNKKKK | 38.63% | 34.25 |
| 14D6 | DDDNDNDND | 54.21% | 34.37 |
| 29K6 | KNNKKNKKK | 4.11% | 36.29 |
| 15K7 | KKNNKKKKK | 8.82% | 37.83 |
| 13K4 | NKNKNNNKK | 6.22% | 39.83 |
| 40D6 | NDNDNDDDD | N/A | N/A |
| 42D6 | NDDDDNDND | N/A | N/A |
| 13D5 | DNDDNNNDD | N/A | N/A |
| 20D7 | DNDNDDDDD | N/A | N/A |
| 45K6 | NKKKKKNNK | N/A | N/A |
| 14K6 | KKKNKNKNK | N/A | N/A |
| 48K5 | NKKNKKNNK | N/A | N/A |
| 48D5 | NDDNDDNND | N/A | N/A |
| 56K6 | NNNKKKKKK | N/A | N/A |
| 1K7 | KKKKNNKKK | N/A | N/A |
| 28K5 | KNKNKKNNK | N/A | N/A |
| 60K5 | NNNKKNKKK | N/A | N/A |
| 6K3 | NNNKKNNNK | N/A | N/A |
| 32K4 | NNNKKNKNK | N/A | N/A |
| 30K5 | KNNNKKNKK | N/A | N/A |
| 5D7 | DDNDNDDDD | N/A | N/A |
| 63D5 | NNNDDDNDD | N/A | N/A |
| 16D6 | DDNNDNDDD | N/A | N/A |
| 48D6 | NDDNDDDND | N/A | N/A |
| 26D7 | NDDNDDDDD | N/A | N/A |
| 28D7 | NNDDDDDDD | N/A | N/A |
| 5D6 | DDDDNDNND | N/A | N/A |
| 8K5 | KKKNKNNNK | N/A | N/A |

The 48 WTA products with the lowest Cts (underlined in Table 8)

were then qPCR amplified using primers for NM_001570-[22348]-01 (screen 3a) and Human B2M Reference Gene (screen 3b), again in a single plate. Since the HB2M Reference gene was not particularly diagnostic, the WTA products were ranked on the basis of lowest Cts for NM_001570-[22348]-01 (see Table 9).

TABLE 9
Third qPCR Screen.

| | | NM_001570- | | Human B2M | |
|---|---|---|---|---|---|
| | | [22348]-01 | | Reference Gene | |
| DNA | Sequence | Effi- | | Effi- | |
| Name | (5′-3′) | ciency | C(t) | ciency | C(t) |
| 61K5 | NNKKKKNNK | 89.73% | 20.62 | 104.79% | 15.96 |
| 24K7 | NKKKKKNKK | 78.90% | 20.64 | 92.38% | 16.63 |
| 3K7 | KKKKNKKNK | 88.21% | 21.08 | 87.42% | 15.8 |
| 11K4 | KNNNKNKNK | 98.83% | 21.13 | 82.51% | 16.02 |
| 25K4 | NNNKNKNKK | 70.12% | 21.15 | 52.40% | 16.72 |
| 41D5 | NDNNNDDDD | 90.41% | 21.49 | 81.38% | 16.33 |
| 16D7 | DNDDNDDDD | 91.62% | 21.49 | 90.46% | 16.96 |
| 54K6 | NNKKKKKNK | 74.28% | 22.69 | 93.76% | 16.04 |
| 15K4 | NKNKNKNNK | 77.62% | 22.96 | 63.86% | 16.89 |
| 6K7 | KKKKKNNKK | 82.93% | 23.27 | 106.46% | 15.47 |
| 55K6 | NNKNKKKKK | 73.93% | 24.07 | 101.32% | 17.43 |
| 19K4 | NKNNKNNKK | 65.68% | 25.39 | 96.74% | 17.19 |
| 8K6 | KKKNNKKNK | 57.01% | 27.69 | 76.50% | 16.27 |
| 27K4 | NNNKNKKNK | 67.81% | 29.01 | 85.25% | 16.99 |
| 13K6 | KKNKKNNKK | 44.87% | 32.06 | 77.82% | 17.06 |
| 18K4 | NKNKKNNNK | 40.16% | 32.56 | 98.27% | 16.43 |
| 21D6 | DNDDNNDDD | 56.41% | 32.89 | 72.69% | 15.72 |
| 9D5 | DDNDDNNND | 51.55% | 33.09 | 112.16% | 15.96 |
| 30K4 | NNNKKNNKK | 57.26% | 33.3 | 76.53% | 16.61 |
| 25D7 | NDDDDDDND | 78.56% | 33.6 | 88.70% | 16.72 |
| 4K3 | NNNKNKNNK | 56.92% | 33.8 | 67.80% | 16.29 |
| 24K5 | KNNKKNNKK | 34.58% | 33.84 | 89.81% | 15.81 |
| 24K4 | NNKKNKNNK | 61.81% | 33.93 | 66.70% | 15.72 |
| 54D6 | NNDDDDDND | 39.75% | 33.98 | 93.20% | 15.81 |
| 54K5 | NNKKNKKNK | 63.39% | 34.13 | 85.45% | 17.44 |
| 54D5 | NNDDNDDND | 62.24% | 34.16 | 75.84% | 15.94 |
| 16K7 | KNKKNKKKK | 40.51% | 34.26 | 79.08% | 18.25 |
| 36K5 | NKKKNKNNK | 50.88% | 34.38 | 108.12% | 15.96 |
| 1D8 | DDDDDNDDD | 37.02% | 34.5 | 76.79% | 15.31 |
| 4K7 | KKKNNKKKK | 58.18% | 35.23 | 104.15% | 15.6 |
| 5K6 | KKKKNKNNK | 37.82% | 35.25 | 83.70% | 16.31 |
| 61D5 | NNDDDDNND | 61.24% | 35.49 | 68.12% | 15.45 |
| 16K5 | KNNKNNKKK | 44.56% | 35.71 | 81.32% | 16.19 |
| 1K9 | KKKKKKKKK | 46.60% | 36.66 | 80.65% | 16.01 |
| 34K5 | NKKNNNKKK | 48.57% | 37.47 | 89.07% | 17.38 |
| 32D4 | NNNDDNDND | 27.18% | 39.28 | 98.38% | 16.09 |
| 65D5 | NNNDDDDND | N/A | N/A | 76.74% | 14.21 |
| 13D7 | DDDNDDDND | N/A | N/A | 50.90% | 14.83 |
| 38K5 | NKNKNKNKK | N/A | N/A | 54.94% | 15.63 |
| 1D9 | DDDDDDDDD | N/A | N/A | 104.78% | 15.64 |
| 22D7 | NDDDNDDDD | N/A | N/A | 58.80% | 15.7 |
| 30D5 | DNNNDDNDD | N/A | N/A | 56.15% | 15.76 |
| 31D5 | DNNNDDDND | N/A | N/A | 84.80% | 16.11 |
| 24D7 | NDDDDDNDD | N/A | N/A | 82.34% | 16.23 |
| 19D7 | DNDDDDDND | N/A | N/A | 70.53% | 16.28 |
| 18D7 | DNDDDDNDD | N/A | N/A | 84.99% | 16.31 |
| 12K4 | KNNNKKNNK | N/A | N/A | 87.09% | 16.93 |
| 27D5 | DNNNDNDDD | N/A | N/A | 96.08% | 17.04 |

The 14 WTA products with the lowest Cts (underlined in Table 9), as well as those amplified with 1K9 and 1D9 primers, were subjected to the fourth qPCR screen (i.e., screens 4a-4f). The 1K9 and 1D9 primers were carried along because current WGA and WTA primers comprise a K9 region and D9 was the first generation attempt at increasing degeneracy relative to K. As before, all reactions were conducted in a single 96-well plate. Table 10 presents the efficiency of amplification and Ct values for each reaction. Of the 16 interrupted N library synthesis primers, five were dropped from further consideration due to either a combination of high Ct for NM_003234 qPCR and/or a lower number of possible WTA amplicons from the human genome. The remaining 11 primers were sorted by Ct for each of the six qPCRs of the fourth screen. At each sorting, a rank number was assigned (1=highest rank, 11 lowest) to each primer. The resulting rank numbers were summed for each primer design (see Table 11). The rank number sums were sorted to provide a ranking of the most successful primers. The process revealed that 9 of the 11 interrupted N primers had similar abilities to provide significant quantities of amplifiable template for the fourth screen.

TABLE 10
Fourth qPCR Screen.

| DNA | Sequence | ATP6V1G1 | | CTNNB1 | | GAPDH | | GPI | | NM_000942 | | NM_003234 | |
|---|---|---|---|---|---|---|---|---|---|---|---|---|---|
| name | (5′-3′) | Eff(%) | C(t)1 | Eff(%) | C(t)2 | Eff(%) | C(t)3 | Eff(%) | C(t)4 | Eff(%) | C(t)5 | Eff(%) | C(t)6 |
| 8K6 | KKKNNKKNK | 84.47 | 19.35 | 83.60 | 18.62 | 88.78 | 15.84 | 90.48 | 18.31 | 97.87 | 17.41 | 83.50 | 20.87 |
| 27K4 | NNNKNKKNK | 49.20 | 20.19 | 63.10 | 19.17 | 81.44 | 14.09 | 84.73 | 18.71 | 86.54 | 16.79 | 77.68 | 22.2 |
| 25K4 | NNNKNKNKK | 69.36 | 22.42 | 66.44 | 18.28 | 73.52 | 15.21 | 62.90 | 18.24 | 91.64 | 17.46 | 58.02 | 21.19 |
| 19K4 | NKNNKNNKK | 62.45 | 21.83 | 83.07 | 19.91 | 56.60 | 15.64 | 82.17 | 18.51 | 70.15 | 17.09 | 71.07 | 20.3 |
| 11K4 | KNNNKNKNK | 33.47 | 25.21 | 87.30 | 19.04 | 73.08 | 15.66 | 78.07 | 17.86 | 88.31 | 18.21 | 64.93 | 20.33 |
| 1D9 | DDDDDDDDD | 61.76 | 18.93 | 74.91 | 19.16 | 72.22 | 14.71 | 69.12 | 19.08 | 109.4 | 18.65 | 8.90 | 30.82 |
| 3K7 | KKKKNKKNK | 61.35 | 19.81 | 98.62 | 20.67 | 91.77 | 15.99 | 80.76 | 19.34 | 105.5 | 16.77 | 76.88 | 20.55 |
| 15K4 | NKNKNKNNK | 59.48 | 23.21 | 77.49 | 19.78 | 83.23 | 15.38 | 57.47 | 18.97 | 80.35 | 17.04 | 75.72 | 20.94 |
| 61K5 | NNKKKKNNK | 82.20 | 20.29 | 75.98 | 19.16 | 76.76 | 14.89 | 79.66 | 19.56 | 85.31 | 17.48 | 48.52 | 32.1 |
| 41D5 | NDNNNDDDD | 94.84 | 20.81 | 76.62 | 20.16 | 83.12 | 15.98 | 84.88 | 18.83 | 98.27 | 19.03 | 84.51 | 21.26 |
| 1K9 | KKKKKKKKK | 86.38 | 23.0 | 66.86 | 24.69 | 79.44 | 17.21 | 72.72 | 19.87 | 78.99 | 19.21 | N/A | N/A |
| 55K6 | NNKNKKKKK | 77.20 | 21.52 | 74.61 | 19.56 | 65.61 | 16.03 | 72.48 | 18.64 | 83.75 | 17.27 | N/A | N/A |
| 24K7 | NKKKKKNKK | 84.59 | 22.12 | 71.78 | 20.23 | 75.70 | 17.81 | 61.66 | 17.29 | 59.52 | 17.34 | 21.89 | 27.98 |
| 54K6 | NNKKKKKNK | 70.42 | 23.57 | 69.26 | 18.07 | 63.88 | 17.43 | 68.88 | 19.92 | 72.48 | 18 | 1.93 | 35.48 |
| 6K7 | KKKKKNNKK | 41.50 | 26.69 | 55.10 | 18.35 | 77.54 | 16.28 | 53.17 | 20.63 | 96.60 | 17.1 | 14.08 | 27.67 |
| 16D7 | DNDDNDDDD | 15.56 | 27.37 | 70.17 | 19.69 | 66.02 | 15.19 | 61.02 | 18.68 | 67.09 | 18.55 | N/A | N/A |

TABLE 11
Ranking of Primers After Fourth qPCR Screen.

| DNA | Sequence | Sort | Sort | Sort | Sort | Sort | Sort | Sort |
|---|---|---|---|---|---|---|---|---|
| Name | (5′-3′) | 1 | 2 | 3 | 4 | 5 | 6 | Sums |
| 8K6 | KKKNNKKNK | 2 | 2 | 8 | 3 | 5 | 4 | 24 |
| 27K4 | NNNKNKKNK | 4 | 6 | 1 | 5 | 2 | 8 | 26 |
| 25K4 | NNNKNKNKK | 8 | 1 | 4 | 2 | 6 | 6 | 27 |
| 19K4 | NKNNKNNKK | 7 | 8 | 6 | 4 | 4 | 1 | 30 |
| 11K4 | KNNNKNKNK | 11 | 3 | 7 | 1 | 8 | 2 | 32 |
| 1D9 | DDDDDDDDD | 1 | 4 | 2 | 8 | 9 | 9 | 33 |
| 3K7 | KKKKNKKNK | 3 | 10 | 10 | 9 | 1 | 3 | 36 |
| 15K4 | NKNKNKNNK | 10 | 7 | 5 | 7 | 3 | 5 | 37 |
| 61K5 | NNKKKKNNK | 5 | 5 | 3 | 10 | 7 | 10 | 40 |
| 41D5 | NDNNNDDDD | 6 | 9 | 9 | 6 | 10 | 7 | 47 |
| 1K9 | KKKKKKKKK | 9 | 11 | 11 | 11 | 11 | 11 | 64 |

In parallel to these experiments, the number of possible human transcriptome derived amplicons resulting from each of the 384 primer designs was determined bioinformatically. Of the nine sequences identified in the four qPCR screens, eight were ranked according the number of potential amplicons produced from the human transcriptome (underlined in Table 11) (1D9 was dropped from further evaluation because of amplicon loss in qPCR screen 3). This analysis identified five sequences (i.e., 11K4, 15K4, 19K4, 25K4, and 27K4), with each producing approximately one million amplicons from the human transcriptome.

### Example 3. Additional Screens to Identify the Exemplary Primers

(a) Amplify Degraded RNA

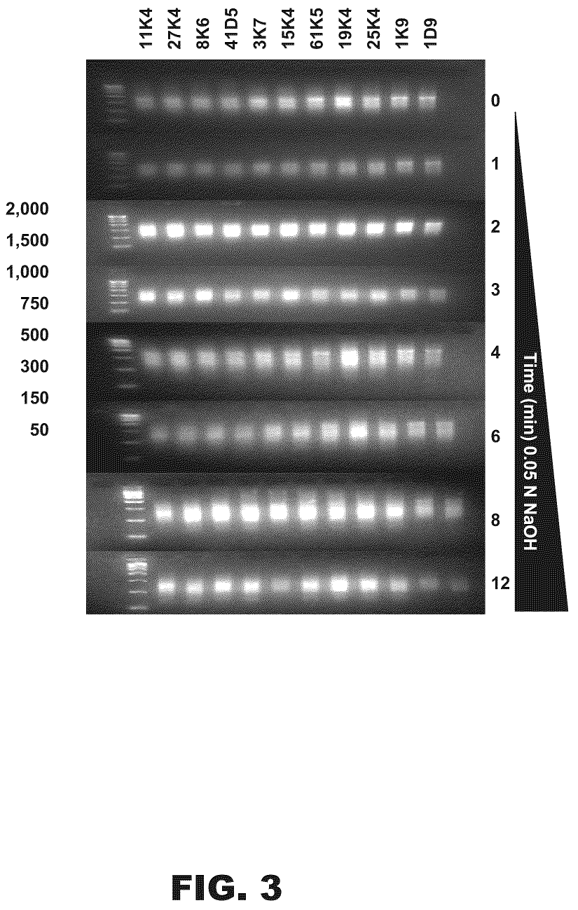

A desirable aspect of the WTA process is the ability to amplify degraded RNAs. The top 9 interrupted N library synthesis primers from screen 4 (see Table 11) plus 1K9 and 1D9 primers were used to amplify NaOH-digested RNAs. Briefly, to 5 μg of liver total RNA in 20 μl of water was added 20 μl of 0.1 M NaOH. The mixture was incubated at 25° C. for 0 minutes to 12 minutes. At times 0, 1, 2, 3, 4, 6, 8 and 12 minutes, 2 μl aliquots were removed and quenched in 100 μl of 10 mM Tris-HCl, pH 7. WTAs were performed similar to those described above. That is, for library synthesis: 2 μl NaOH-digested RNA, 2 μl of 5 μM of a library synthesis primer, heat 70° C. for 5 min, add 4 μl of 2× MMLV buffer, 10 U/μl MMLV, and 1 mM dNTPs; incubate at 42° C. for 15 minutes; and dilute with 30 μl of H₂O. For amplification: 8 μl of diluted library, 12 μl of amplification mix (2× SYBR® Green JUMPSTART™ Taq READYMIX™ and 5 μM universal primer). Analysis of the WTA products by agarose gel electrophoresis revealed that all except 1K9 and 1D9 library synthesis primers produced relatively high levels of WTA amplicons (see FIG. 3).
(b) WTA Screens

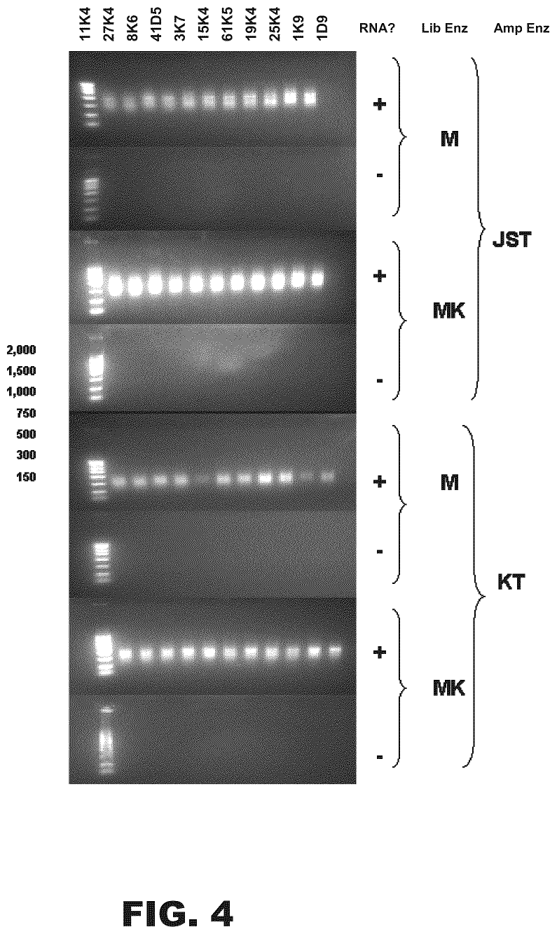

Another desirable feature of an ideal library synthesis primer is minimal or no primer dimer formation. The 11 interrupted N primers used in the above-described degraded RNA experiment were subjected to WTA except in the absence of template. Library synthesis was also performed in the presence of either MMLV reverse transcriptase or both MMLV and Klenow exo-minus DNA polymerase. Library amplification was also catalyzed by either JUMPSTART™ Taq or KLENTAQ® (Sigma-Aldrich). FIG. 4 reveals that synthesis with the combination of MMLV and Klenow exo-minus DNA polymerase and amplification with JUMPSTART™ Taq DNA polymerase provided higher levels of amplicons. Furthermore, this experiment revealed that primer dimer formation was not a significant problem with any of these 11 library synthesis primers (see gels without RNA template).
(c) Final Selection

The preferred library synthesis primers would be primers that provide a maximum number of amplicons without a loss of sensitivity due to intermolecular and/or intramolecular primer specific interactions (e.g., primer dimers). Thus, the qPCR culling experiments, the primer dimer analyses, and the bioinformatics analyses revealed five interrupted N sequences that satisfied these requirements. That is, five sequences (i.e., 11K4, 15K4, 19K4, 25K4, and 27K4) that when used for library synthesis yielded WTA products that provided amplifiable template for all qPCR screens, yielded minimal quantities of primer dimers in the absence of template, and were capable of producing at least a million WTA amplicons from the human transcriptome.

Although one of these preferred sequences could be randomly selected for use as a library synthesis primer, it was reasoned that a mixture of some or all of these sequences may be preferable. Conversely, a mixture of some or all of them could also permit detrimental primer-primer interactions. These possibilities were investigated by performing WTA in which the libraries were synthesized using individual primers or a mixture of some or all five of the preferred primers, as well as primers comprising K9, D9, or N9 sequences. Potentially detrimental interactions were examined by performing library synthesis with high concentrations of the library synthesis primer(s). Thus, standard WTA reactions library were performed in the presence of 10 μM, 2 μM, 0.4 μM or 0.08 μM of the library synthesis primers. WTA products were assayed by agarose gel electrophoresis. WTA products were also analyzed with SYBR® green mediated qPCR amplification using NM_001570 primers (SEQ ID NOs:33 and 34).

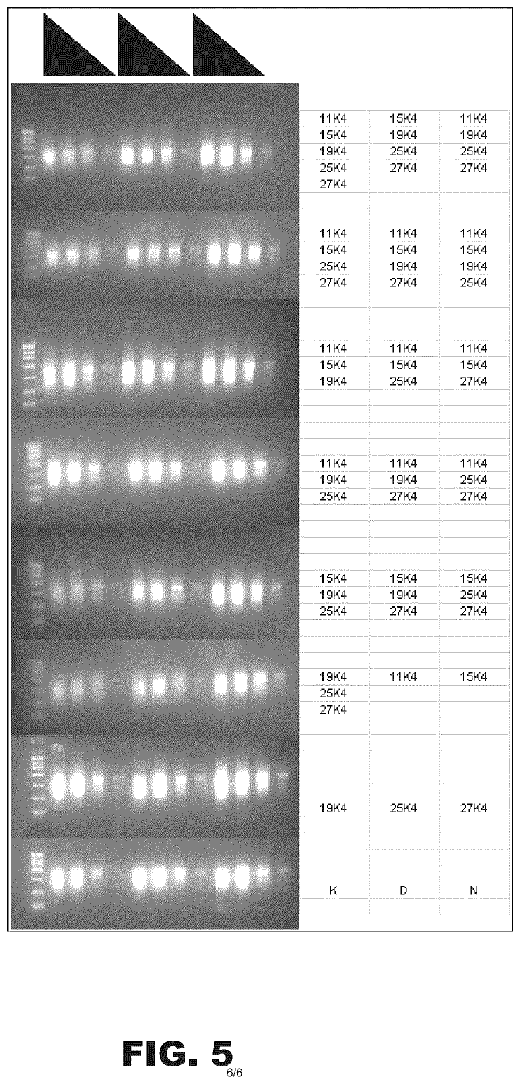

As shown in FIG. 5, the yield of WTA products was dependent upon the concentration of the library synthesis primer(s). Furthermore, evidence of primer dimers was present only at the highest concentration of the N9 primer (see N lanes). The possibility of primer interactions was estimated by calculating the delta Cts from qPCR for each primer/primer combination. That is, the difference in Ct between 10 μM and 2 μM, between 2 μM and 0.4 μM, and between 0.4 μM and 0.08 μM. A negative delta Ct was interpreted as a detrimental primer-primer interaction. It was found that 15K4 alone had modest detrimental interactions at high concentrations, while almost any combination that contained 15K4 and 19K4 was also significantly detrimental. Additionally, the combination of 19K4 and 25K4 also showed a negative interaction.

TABLE 12
qPCR using individual primers or primer combinations.

| Primers* | Ct(1)** | Ct(2)** | Ct(3)** | Ct(4)** | ΔCt(2-1) | ΔCt(3-2) | ΔCt(4-3) |
|---|---|---|---|---|---|---|---|
| 11, 15, 19, 25, 27 | 22.11 | 22.63 | 23.61 | 25.02 | 0.52 | 0.98 | 1.41 |
| 15, 19, 25, 27 | 22.44 | 24.72 | 22.91 | 26.61 | 2.28 | −1.81 | 3.7 |
| 11, 19, 25, 27 | 21.7 | 22.73 | 24.28 | 25.97 | 1.03 | 1.55 | 1.69 |
| 11, 15, 25, 27 | 23.06 | 23.26 | 23.34 | 28.91 | 0.2 | 0.08 | 5.57 |
| 11, 15, 19, 27 | 23.58 | 23.68 | 24.16 | 24.35 | 0.1 | 0.48 | 0.19 |
| 11, 15, 19, 25 | 24.73 | 23.34 | 26.0 | 25.82 | −1.39 | 2.66 | −0.18 |
| 11, 15, 19 | 23.78 | 22.82 | 24.51 | 28.36 | −0.96 | 1.69 | 3.85 |
| 11, 15, 25 | 23.18 | 23.73 | 28.05 | 29.4 | 0.55 | 4.32 | 1.35 |
| 11, 15, 27 | 22.73 | 23.03 | 23.07 | 27.99 | 0.3 | 0.04 | 4.92 |
| 11, 15, 27 | 22.28 | 23.7 | 22.25 | 27.15 | 1.42 | −1.45 | 4.9 |
| 11, 19, 25 | 19.67 | 22.47 | 22.68 | 27.62 | 2.8 | 0.21 | 4.94 |
| 11, 19, 27 | 18.67 | 20.09 | 25.11 | 25.49 | 1.42 | 5.02 | 0.38 |
| 11, 25, 27 | 22.1 | 23.45 | 19.93 | 22.12 | 1.35 | −3.52 | 2.19 |
| 15, 19, 25 | 24.21 | 21.51 | 22.65 | 25.06 | −2.7 | 1.14 | 2.41 |
| 15, 25, 27 | 23.42 | 23.71 | 23.65 | 24.96 | 0.29 | −0.06 | 1.31 |
| 19, 25, 27 | 23.42 | 22.36 | 23.21 | 27.16 | −1.06 | 0.85 | 3.95 |
| 11 | 23.17 | 24.09 | 22.8 | 27.86 | 0.92 | −1.29 | 5.06 |
| 15 | 23.5 | 22.06 | 23.32 | 24.78 | −1.44 | 1.26 | 1.46 |
| 19 | 23.73 | 23.79 | 23.82 | 28.97 | 0.06 | 0.03 | 5.15 |
| 25 | 23.25 | 23.0 | 24.0 | 24.8 | −0.25 | 1.0 | 0.8 |
| 27 | 23.67 | 23.27 | 23.74 | 27.17 | −0.4 | 0.47 | 3.43 |
| K | 22.69 | 22.27 | 22.3 | 27.98 | −0.42 | 0.03 | 5.68 |
| D | 23.74 | 23.73 | 24.43 | 28.33 | −0.01 | 0.7 | 3.9 |
| N | 24.29 | 24.78 | 21.59 | 24.98 | 0.49 | −3.19 | 3.39 |

*11 = 11K4 primer, 15 = 15K4 primer, 19 = 19K4 primer, 25 = 25K4 primer, 27 = 27K4 primer.
**1 = 10 μM, 2 = 2 μM, 3 = 0.4 μM, 4 = 0.08 μM.

Aside from any possible negative impact the combination of primers might have, their ability to prime divergent sequences was probed by pair-wise alignment of the individual sequences. The 5 interrupted N were aligned so as to have the greatest number of Ns overlapping among the primers (see Table 13). Furthermore, pair-wise K-N mismatches were tallied for each possible pairing (see Table 14).

TABLE 13
Pair-wise Alignment.

| Name | Sequence (5′-3′) |
|---|---|
| 11K4 | K N N N K N K N K |
| 15K4 | N K N K N K N N K |
| 19K4 | N K N N K N N K K |
| 25K4 | N N N K N K N K K |
| 27K4 | N N N K N K K N K |

TABLE 14
Mismatches

| | 11K4 | 15K4 | 19K4 | 25K4 | 27K4 |
|---|---|---|---|---|---|
| 11K4 | | 2 | 3 | 0 | 2 |
| 15K4 | | | 2 | 2 | 2 |
| 19K4 | | | | 3 | 3 |
| 25K4 | | | | | 2 |
| 27K4 | | | | | |

These analyses revealed that the greatest divergence within this set of primers was with 11K4, 19K4 and 27K4 primers. Thus, maximum priming divergence with minimal primer interaction occurred with the mixture of primers comprising 11K4 (i.e., KNNNKNKNK), 19K4 (i.e., NKNNKNNKK), and 27K4 (i.e., NNNKNKKNK).

1. A plurality of degenerate oligonucleotides, each oligonucleotide having a sequence selected from KNNNKNKNK, NKNNKNNKK, or NNNKNKKNK, wherein
N is a 4-fold degenerate nucleotide selected from adenosine (A), cytidine (C), guanosine (G), or thymidine/uridine (T/U); and
K is G or T/U.

2. The plurality of degenerate oligonucleotides of claim 1, wherein each oligonucleotide further comprises a sequence of non-degenerate nucleotides at the 5′ end, the non-degenerate sequence being constant among the plurality of oligonucleotides, and the constant non-degenerate sequence being about 14 nucleotides to about 24 nucleotides in length.


SEQUENCE LISTING (53 sequences)

SEQ ID NO: 1 - DNA, 44 bp, synthetic construct
  source 1..44: mol_type = other DNA; note = Derived from Homo sapiens
gtaggttgaggataggagggttaggttttttttttttttttttt

SEQ ID NO: 2 - DNA, 34 bp, synthetic construct
  source 1..34: mol_type = other DNA; note = Derived from Homo sapiens
gtaggttgaggataggagggttaggddddddddd

SEQ ID NO: 3 - DNA, 25 bp, synthetic construct
  source 1..25: mol_type = other DNA; note = Derived from Homo sapiens
gtaggttgaggataggagggttagg

SEQ ID NO: 4 - DNA, 20 bp, synthetic construct
  source 1..20: mol_type = other DNA; note = Derived from Homo sapiens
tgcttagacccgtagtttcc

SEQ ID NO: 5 - DNA, 21 bp, synthetic construct
  source 1..21: mol_type = other DNA; note = Derived from Homo sapiens
cttgacaaaatgctgtgttcc

SEQ ID NO: 6 - DNA, 20 bp, synthetic construct
  source 1..20: mol_type = other DNA; note = Derived from Homo sapiens
cgtttaattctgtggccagg

SEQ ID NO: 7 - DNA, 20 bp, synthetic construct
  source 1..20: mol_type = other DNA; note = Derived from Homo sapiens
agccaagtaccccgactacg

SEQ ID NO: 8 - DNA, 25 bp, synthetic construct
  source 1..25: mol_type = other DNA; note = Derived from Homo sapiens
tgttaacaatttgcataacaaaagc

SEQ ID NO: 9 - DNA, 25 bp, synthetic construct
  source 1..25: mol_type = other DNA; note = Derived from Homo sapiens
tgattaatttgcgagactaactttg

SEQ ID NO: 10 - DNA, 23 bp, synthetic construct
  source 1..23: mol_type = other DNA; note = Derived from Homo sapiens
gtttcgaatcccaggaattaagc

SEQ ID NO: 11 - DNA, 23 bp, synthetic construct
  source 1..23: mol_type = other DNA; note = Derived from Homo sapiens
cacaatcagcaacaaaatcatcc

SEQ ID NO: 12 - DNA, 20 bp, synthetic construct
  source 1..20: mol_type = other DNA; note = Derived from Homo sapiens
gcaaacaaagcatgcttcaa

SEQ ID NO: 13 - DNA, 20 bp, synthetic construct
  source 1..20: mol_type = other DNA; note = Derived from Homo sapiens
ttctcccagctttgagacgt

SEQ ID NO: 14 - DNA, 25 bp, synthetic construct
  source 1..25: mol_type = other DNA; note = Derived from Homo sapiens
tatttaaaatgtgggcaagatatca

SEQ ID NO: 15 - DNA, 25 bp, synthetic construct
  source 1..25: mol_type = other DNA; note = Derived from Homo sapiens
tggtgtaaataaagaccttgctatc

SEQ ID NO: 16 - DNA, 21 bp, synthetic construct
  source 1..21: mol_type = other DNA; note = Derived from Homo sapiens
tttgttacttgctaccctgag

SEQ ID NO: 17 - DNA, 21 bp, synthetic construct
  source 1..21: mol_type = other DNA; note = Derived from Homo sapiens
caaccatcatcttccacagtc

SEQ ID NO: 18 - DNA, 20 bp, synthetic construct
  source 1..20: mol_type = other DNA; note = Derived from Homo sapiens
agaccacaccagaaaccctg

SEQ ID NO: 19 - DNA, 22 bp, synthetic construct
  source 1..22: mol_type = other DNA; note = Derived from Homo sapiens
gaattttggtttcttgctttgg

SEQ ID NO: 20 - DNA, 23 bp, synthetic construct
  source 1..23: mol_type = other DNA; note = Derived from Homo sapiens
ccagggttcgaatctcagtctta

SEQ ID NO: 21 - DNA, 23 bp, synthetic construct
  source 1..23: mol_type = other DNA; note = Derived from Homo sapiens
gatttctaaacttacggccccac

SEQ ID NO: 22 - DNA, 25 bp, synthetic construct
  source 1..25: mol_type = other DNA; note = Derived from Homo sapiens
aaagagtgtcttgtcttgacttatc

SEQ ID NO: 23 - DNA, 25 bp, synthetic construct
  source 1..25: mol_type = other DNA; note = Derived from Homo sapiens
ttatctgagcccttaatagtaaatc

SEQ ID NO: 24 - DNA, 20 bp, synthetic construct
  source 1..20: mol_type = other DNA; note = Derived from Homo sapiens
aatcaaaaggccaacagtgg

SEQ ID NO: 25 - DNA, 21 bp, synthetic construct
  source 1..21: mol_type = other DNA; note = Derived from Homo sapiens
ttcagtgttaatggagccagg

SEQ ID NO: 26 - DNA, 20 bp, synthetic construct
  source 1..20: mol_type = other DNA; note = Derived from Homo sapiens
tctcagagcagagtttgggc

SEQ ID NO: 27 - DNA, 19 bp, synthetic construct
  source 1..19: mol_type = other DNA; note = Derived from Homo sapiens
cctgcacttggacctgacc

SEQ ID NO: 28 - DNA, 22 bp, synthetic construct
  source 1..22: mol_type = other DNA; note = Derived from Homo sapiens
gtggtgtgttgggtgtgtttgg

SEQ ID NO: 29 - DNA, 20 bp, synthetic construct
  source 1..20: mol_type = other DNA; note = Derived from Homo sapiens
ctggaacggtgaaggtgaca

SEQ ID NO: 30 - DNA, 23 bp, synthetic construct
  source 1..23: mol_type = other DNA; note = Derived from Homo sapiens
aagggacttcctgtaacaatgca

SEQ ID NO: 31 - DNA, 25 bp, synthetic construct
  source 1..25: mol_type = other DNA; note = Derived from Homo sapiens
ctcagttggtgtgcccaaagtttca

SEQ ID NO: 32 - DNA, 24 bp, synthetic construct
  source 1..24: mol_type = other DNA; note = Derived from Homo sapiens
tagcagagttacttctaagggttc

SEQ ID NO: 33 - DNA, 21 bp, synthetic construct
  source 1..21: mol_type = other DNA; note = Derived from Homo sapiens
gatcatcctgaactggaaacc

SEQ ID NO: 34 - DNA, 24 bp, synthetic construct
  source 1..24: mol_type = other DNA; note = Derived from Homo sapiens
gcctttcttacagaagctgccaaa

SEQ ID NO: 35 - DNA, 22 bp, synthetic construct
  source 1..22: mol_type = other DNA; note = Derived from Homo sapiens
cggcatcttcaaacctccatga

SEQ ID NO: 36 - DNA, 29 bp, synthetic construct
  source 1..29: mol_type = other DNA; note = Derived from Homo sapiens
gcctgccgtgtgaaccatgtgactttgtc

SEQ ID NO: 37 - DNA, 20 bp, synthetic construct
  source 1..20: mol_type = other DNA; note = Derived from Homo sapiens
tggacaacctcttggctttt

SEQ ID NO: 38 - DNA, 20 bp, synthetic construct
  source 1..20: mol_type = other DNA; note = Derived from Homo sapiens
taaaatgccactccacagca

SEQ ID NO: 39 - DNA, 20 bp, synthetic construct
  source 1..20: mol_type = other DNA; note = Derived from Homo sapiens
ttgaaaatccagcgtggaca

SEQ ID NO: 40 - DNA, 20 bp, synthetic construct
  source 1..20: mol_type = other DNA; note = Derived from Homo sapiens
tcgagtcattgcatactgtc

SEQ ID NO: 41 - DNA, 19 bp, synthetic construct
  source 1..19: mol_type = other DNA; note = Derived from Homo sapiens
gaaggtgaaggtcggagtc

SEQ ID NO: 42 - DNA, 20 bp, synthetic construct
  source 1..20: mol_type = other DNA; note = Derived from Homo sapiens
gaagatggtgatgggatttc

SEQ ID NO: 43 - DNA, 20 bp, synthetic construct
  source 1..20: mol_type = other DNA; note = Derived from Homo sapiens
aggctgctgccacataaggt

SEQ ID NO: 44 - DNA, 20 bp, synthetic construct
  source 1..20: mol_type = other DNA; note = Derived from Homo sapiens
ccaaggctccaagcatgaat

SEQ ID NO: 45 - DNA, 22 bp, synthetic construct
  source 1..22: mol_type = other DNA; note = Derived from Homo sapiens
caaagtcaccgtcaaggtgtat

SEQ ID NO: 46 - DNA, 25 bp, synthetic construct
  source 1..25: mol_type = other DNA; note = Derived from Homo sapiens
ggaacagtctttccgaagagaccaa

SEQ ID NO: 47 - DNA, 25 bp, synthetic construct
  source 1..25: mol_type = other DNA; note = Derived from Homo sapiens
cagactaacaacagatttcgggaat

SEQ ID NO: 48 - DNA, 24 bp, synthetic construct
  source 1..24: mol_type = other DNA; note = Derived from Homo sapiens
gaggaagtgatactccactctcat

SEQ ID NO: 49 - DNA, 20 bp, synthetic construct
  source 1..20: mol_type = other DNA
  misc_difference 1..20: note = This sequence may encompass 2-20 residues
bbbbbbbbbbbbbbbbbbbb

SEQ ID NO: 50 - DNA, 20 bp, synthetic construct
  source 1..20: mol_type = other DNA
  misc_difference 1..20: note = This sequence may encompass 2-20 residues
dddddddddddddddddddd

SEQ ID NO: 51 - DNA, 20 bp, synthetic construct
  source 1..20: mol_type = other DNA
  misc_difference 1..20: note = This sequence may encompass 2-20 residues
hhhhhhhhhhhhhhhhhhhh

SEQ ID NO: 52 - DNA, 20 bp, synthetic construct
  source 1..20: mol_type = other DNA
  misc_difference 1..20: note = This sequence may encompass 2-20 residues
vvvvvvvvvvvvvvvvvvvv

SEQ ID NO: 53 - DNA, 19 bp, synthetic construct
  source 1..19: mol_type = other DNA
ttttttttttttttttttt